(19) United States
(12) Patent Application Publication
(10) Pub. No.: US 2026/0274945 A1
(43) Pub. Date: Sep. 17, 2026

(54) Antibodies To Cell Adhesion Molecule-Related/Down-Regulated By Oncogenes (CDON) And Uses Thereof

(71) Applicant: Institute For Cancer Research d/b/a The Research Institute of Fox Chase Cancer Center, Philadelphia, PA (US)

(72) Inventors: Denise C. Connolly, Philadelphia, PA (US); Alana M. O'Reilly, Philadelphia, PA (US); Tiffiney R. Hartman, Philadelphia, PA (US); Valerie L. Sodi, Philadelphia, PA (US)

(73) Assignee: Institute For Cancer Research d/b/a The Research Institute of Fox Chase Cancer Center, Philadelphia, PA (US)

(21) Appl. No.: 19/573,385

(22) Filed: Mar. 20, 2026

Related U.S. Application Data
(62) Division of application No. 17/430,227, filed on Feb. 12, 2020, now abandoned.
(60) Provisional application No. 62/806,122, filed on Feb. 15, 2019.

(51) Int. Cl. C07K 16/28; G01N 33/57545

(52) U.S. Cl. CPC C07K 16/2803; G01N 33/57545; C07K 2317/34; C07K 2317/73; C07K 2317/76

(57) ABSTRACT

The present disclosure provides antibodies specifically binding N-terminal or C-terminal regions of Cell Adhesion Molecule-Related/Down-Regulated By Oncogenes (CDON) polypeptide, methods of making the same, and methods of treating humans having tumors by administering the antibody.

### REFERENCE TO GOVERNMENT GRANTS

This invention was made with government support under Grant/Contract Number HD065800 awarded by the National Institutes of Health, and Grant/Contract Number W81XWH-16-1-0142 awarded by U.S. Army Medical Research and Materiel Command. The government has certain rights in the invention.

### REFERENCE TO SEQUENCE LISTING

The instant application contains a Sequence Listing which has been submitted electronically in XML format and is hereby incorporated by reference in its entirety. Said XML copy, created on Apr. 26, 2026, is named J4074-00202_SL.xml, and is 8,331 bytes in size.

### FIELD

The present disclosure is directed, in part, to antibodies specifically binding N-terminal or C. terminal regions of Cell Adhesion Molecule-Related/Down-Regulated By Oncogenes (CDON) polypeptide, and to the methods of making and using the same.

### BACKGROUND

Malignant tumors (cancers) are the second leading cause of death in the United States after heart disease (Boring et al., CA Cancel J. Clin., 1993, 43, 7). In a cancerous state, a cell proliferates under conditions in which normal cells would not grow. Cancer is characterized by the increase in the number of abnormal, or neoplastic, cells derived from a normal tissue which proliferate to form a tumor mass. Cancer can also spread through the invasion of adjacent tissues by these neoplastic tumor cells, and the generation of malignant cells which disseminate locally and eventually spread via the blood or lymphatic system to regional lymph nodes and to distant sites via a process called metastasis. The appearance of metastatic lesions is dependent on cell-cell interactions of cancer cells with normal mesothelium, epithelium, and endothelium on the surface of normal tissues and organs. These interactions are mediated by cell adhesion molecules which thus play a significant role in cancer progression and metastasis. Inhibition of these interactions represent a therapeutically useful target for attenuation of metastasis. Cancer manifests itself in a wide variety of forms, characterized by different degrees of invasiveness and aggressiveness.

Pancreatic cancer is the 4th leading cause of cancer deaths in the United States with a 5-year survival rate of less than 7% and a median survival of only 3 to 6 months. Most pancreatic ductal adenocarcinomas (PDACs) are diagnosed at a late stage, are not surgically resectable, and respond poorly to chemotherapy. Thus, discovery of methods for detection of early stage disease is critical for meaningful improvements in patient outcomes. PDAC develops from early, non-invasive neoplastic lesions, the most common of which are pancreaticintraepithelial neoplasia (PanINs). PanINs are very common with 26% of patients with non-cancerous pancreatic disease exhibiting these lesions. Only 1% of individual PanINs progress to invasive cancer, suggesting the existence of critical inhibitory mechanisms that maintain the benign state. Currently, mechanisms that prevent or trigger progression of PanINs to adenocarcinoma are completely unknown.

Early detection of pancreatic cancer is challenging due to lack of specific symptoms, insufficient serological biomarkers, and the difficulty of clinical examination of the pancreas. Very little is known about the underlying signaling mechanisms that regulate step-wise transition states that drive progression from normal pancreatic epithelium through benign and premalignant neoplasms to carcinomas. Sonic Hedgehog (SHH) signaling has been implicated in this developmental process, with aberrant SHH expression observed in the earliest PanINs.

Ovarian cancer ranks 11th in new cancer diagnosis and the 5th leading cause of cancer associated death in women in the United States. The high mortality rate is reflective of the fact that most cases of ovarian carcinoma (OC) are diagnosed at advanced stage (Stage III/IV). In the absence of effective methods for prevention or early detection, the incidence of OC has remained the same over the past several decades. After diagnosis, OC patients undergo aggressive cytoreductive surgery and are treated with standard combination chemotherapy consisting of platinum and taxane agents. Most patients respond well to this approach, but the majority will eventually experience disease recurrence. One of the primary reasons for the high rate of recurrence is that OC patients are diagnosed when their cancers have already spread beyond the primary tumor and are widely dissmeninated in the abdominal cavity making complete surgical removal of the tumor unlikely. In the majority of cases, recurrent OC ultimately becomes resistant to standard cytotoxic chemotherapy and there currently are no curative treatment options for patients who experience recurrent drug-resistant disease.

Unlike tumors that spread via entry into the bloodstream, OC dissemination primarily occurs by sloughing or shedding of tumor cells from the primary tumor, aggregation and survival of these cells in the peritoneal fluid, followed by attachment to and colonization of the peritoneal surface including, but not limited to, omentum, mesentery and colon. A significant proportion of OC patients develop ascites, an accumulation of fluid and malignant cells in the peritoneal cavity that further facilitates survival and spread of tumor. In patients with ascites, the aggregation and survival of drug-resistant cells as multicellular tumor cell clusters or spheroids provides a potent reservoir of microscopic disease that can seed new tumor growth. Formation of multicellular tumor spheroids relies on intercellular attachments allowing cells to cluster and survive detachment-mediated cell death by anoikis.

Cell Adhesion Molecule-Related/Down-Regulated By Oncogenes (CDON) polypeptide is a type I cell surface receptor glycoprotein, containing ectodomain (EC domain) structural features, such as four Ig repeats and three Fibronectin (FN) type III repeats in the ~960 amino acid extracellular domain, a 20 amino acid transmembrane (TM) domain, and an ~300 amino acid intracellular domain (IC domain) with no identifiable motifs. This domain architecture is closely related to that of axon guidance receptors of the Robo and DCC (deleted in colorectal cancer) families. CDON is a well-documented SHH binding protein that acts as an SHH effector in receiving cells. Interactions with cadherins are with the FN domain 1, the HH binding domain is in the most membrane proximal FN domain (FN3) and signaling via p38MAPK, CDC42 and AKT occurs via the cytoplasmic domain.

Boi, the Drosophila homolog of CDON plays a fundamental role in regulation of epithelial stem cell proliferation in the Drosophila ovary. Boi binds to and sequesters hedgehog (HH) in producing cells, releasing it in response to environmental cues to promote stem cell proliferation.

During embryonic development, CDON is expressed in the musculoskeletal and central nervous systems and in areas of proliferation and differentiation. CDON has further been associated with myogenic differentiation (Kang et al., EMBO J., 2002, 21, 114-124) and macrophage defects (PCT Publication WO/2006/132788). Expression of CDON in myoblast cell lines is downregulated by the ras oncogene, and forced re-expression of either CDON can override ras-induced inhibition of myogenic differentiation (Kang et al., J. Cell Biol., 1998, 143, 403-413; and Kang et al., EMBO J., 2002, 21, 114-124). The promyogenic properties of CDON were further shown to be present in the human rhabdomyosarcoma cell line, RD. Stable overexpression of CDON in RD cells led to enhanced expression of two markers of muscle cell differentiation, troponin T and myosin heavy chain, and to increased formation of elongated, myosin heavy chain-positive myotubes. It has further been suggested that CDON plays a role in the inverse relationship between differentiation and transformation of cells in the skeletal muscle lineage (Wegorzewska et al., Mol. Carcinogenesis, 2003, 37, 1-4). In addition, CDON functions as a receptor for SHH and, in some cases, behaves as an SHH dependence receptor, where it actively triggers apoptosis in the absence of SHH. The pro-apoptotic activity of unbound CDON requires a proteolytic cleavage in its intracellular domain, allowing the recruitment and activation of caspase-9.

A central role that CDON appears to play in cell adhesion and several cancer-related signaling pathways suggests that it is a promising therapeutic target. However, this potential of CDON remains unexplored. Accordingly, there is a need for therapeutic and diagnostic tools that can assess and modulate the function of CDON.

### SUMMARY

The present disclosure provides an isolated antibody, or antigen-binding fragment thereof, specific for Cell Adhesion Molecule-Related/Down-Regulated by Oncogenes (CDON) polypeptide, wherein the antibody, or antigen-binding fragment thereof, specifically binds: a) a polypeptide consisting of amino acids at positions corresponding to positions 1 to 200 according to SEQ ID NO: 1; or b) a polypeptide consisting of amino acids at positions corresponding to positions 1000 according to 1287 according to SEQ ID NO: 1.

The present disclosure also provides an isolated antibody, or antigen-binding fragment thereof, wherein the antibody, or antigen-binding fragment thereof, specifically binds: a) a polypeptide consisting of amino acids at positions corresponding to positions 100 to 200 according to SEQ ID NO: 1; or b) a polypeptide consisting of amino acids at positions corresponding to positions 1200 to 1287 according to SEQ ID NO:1.

The present disclosure also provides an isolated antibody, or antigen-binding fragment thereof, wherein the antibody, or antigen-binding fragment thereof, specifically binds: a) a polypeptide consisting of amino acids at positions corresponding to positions 140 to 170 according to SEQ ID NO: 1; or b) a polypeptide consisting of amino acids at positions corresponding to positions 1250 to 1287 according to SEQ ID NO:1.

The present disclosure also provides an isolated antibody, or antigen-binding fragment thereof, wherein the antibody, or antigen-binding fragment thereof, specifically binds: a polypeptide consisting of the amino acid sequence RVPESNPKAEVRYKIRGK (SEQ ID NO:2), a polypeptide consisting of the amino acid sequence GIPLDSPTEVLQQP RET (SEQ ID NO:3), a polypeptide consisting of the amino acid sequence VLGDFGSSTTKHVITAEE (SEQ ID NO:4), or a polypeptide consisting of the amino acid sequence KIRGKWLEHSTENY (SEQ ID NO:5).

The present disclosure also provides a method of making an antibody specific for Cell Adhesion Molecule-Related/Down-Regulated By Oncogenes (CDON) polypeptide, comprising immunizing an animal with an immunogenic form of the isolated peptide selected from: a) the polypeptide consisting of amino acid residues 1 to 200 according to SEQ ID NO: 1, or a fragment thereof, and/or b) the polypeptide consisting of amino acid residues 1000 to 1287 according to SEQ ID NO: 1, or a fragment thereof.

The present disclosure also provides a method of making an antibody specific for Cell Adhesion Molecule-Related/Down-Regulated by Oncogenes (CDON) protein, comprising immunizing an animal with: a) a polypeptide consisting of amino acids at positions corresponding to positions 1 to 200 according to SEQ ID NO: 1; and/or b) a polypeptide consisting of amino acids at positions corresponding to positions 1000 to 1287 according to SEQ ID NO: 1.

The present disclosure also provides a method of making an antibody specific for Cell Adhesion Molecule-Related/Down-Regulated by Oncogenes (CDON) protein, comprising immunizing an animal with: a) a polypeptide consisting of the amino acid sequence RVPESNPKAEVRYKIRGK (SEQ ID NO:2); b) a polypeptide consisting of the amino acid sequence GIPLDSPTEVLQQPRET (SEQ ID NO:3); c) a polypeptide consisting of the amino acid sequence VLGDFGSSTTKHVITAEE (SEQ ID NO:4); and/or d) a polypeptide consisting of the amino acid sequence KIRGKWLEHSTENY (SEQ ID NO:5).

The present disclosure also provides a method of detecting the presence or absence of a tumor in a mammal comprising: a) contacting a tissue or cell sample obtained from the mammal with an antibody, or antigen-binding fragment thereof, that specifically binds Cell Adhesion Molecule-Related/Down-Regulated by Oncogenes (CDON) polypeptide, wherein the antibody, or antigen-binding fragment thereof, specifically binds: i) a polypeptide consisting of amino acids at positions corresponding to positions 1 to 200 according to SEQ ID NO: 1; and/or ii) a polypeptide consisting of amino acids at positions corresponding to positions 1000 to 1287 according to SEQ ID NO: 1; b) detecting the presence or absence of a complex between the antibody, or antigen-binding fragment thereof, and a CDON polypeptide in the sample; and c) comparing the formation or lack or formation of the complex in the sample with a control sample, wherein the formation of a greater amount of the complex in the sample compared to the control sample indicates the presence of a tumor in the mammal, and wherein the formation of an equal amount or lesser amount of the complex in the sample compared to the control sample indicates the absence of a tumor in the mammal.

The present disclosure also provides a method for determining the presence or absence of Cell Adhesion Molecule-Related/Down-Regulated by Oncogenes (CDON) polypeptide in a human comprising: a) administering to the human an antibody, or antigen-binding fragment thereof, that specifically binds the CDON polypeptide, wherein the antibody, or antigen-binding fragment thereof, specifically binds: i) a polypeptide consisting of amino acids at positions corresponding to positions 1 to 200 according to SEQ ID NO: 1; and/or ii) a polypeptide consisting of amino acids at positions corresponding to positions 1000 to 1287 according to SEQ ID NO: 1; wherein the antibody, or antigen-binding fragment thereof, is labeled with a detectable label; and b) externally scanning the human for localization of the labeled antibody, or antigen-binding fragment thereof.

The present disclosure also provides a method for determining the expression levels of Cell Adhesion Molecule-Related/Down-Regulated By Oncogenes (CDON) polypeptide in a patient suspected of having a tumor, comprising: a) administering to the patient an antibody that binds to CDON polypeptide, or an antigen-binding fragment thereof, wherein the antibody or the antigen-binding fragment thereof, is labeled with a detectable label; and b) externally scanning the patient for localization of the label; wherein the antibody, or antigen-binding fragment thereof, specifically binds an isolated peptide selected from: (i) the polypeptide consisting of amino acid residues 1 to 200 according to SEQ ID NO: 1; and (ii) the polypeptide consisting of amino acid residues 1000 to 1287 according to SEQ ID NO:1.

The present disclosure also provides a method for treating a human having a tumor comprising administering to the human in need thereof an antibody, or antigen-binding fragment thereof, that specifically binds Cell Adhesion Molecule-Related/Down-Regulated by Oncogenes (CDON) polypeptide, wherein the antibody, or antigen-binding fragment thereof, specifically binds: i) a polypeptide consisting of amino acids at positions corresponding to positions 1 to 200 according to SEQ ID NO: 1; or ii) a polypeptide consisting of amino acids at positions corresponding to positions 1000 to 1287 according to SEQ ID NO:1.

The present disclosure also provides an antibody, or antigen-binding fragment thereof, that specifically binds to Cell Adhesion Molecule-Related/Down-Regulated by Oncogenes (CDON) polypeptide for use in a method of treating cancer.

The present disclosure also provides an antibody, or antigen-binding fragment thereof, that specifically binds to Cell Adhesion Molecule-Related/Down-Regulated by Oncogenes (CDON) polypeptide for use in the preparation of a medicament for treating cancer.

The present disclosure also provides a use of an antibody, or antigen-binding fragment thereof, that specifically binds to Cell Adhesion Molecule-Related/Down-Regulated by Oncogenes (CDON) polypeptide in a method of treating cancer.

The present disclosure also provides a use of an antibody, or antigen-binding fragment thereof, that specifically binds to Cell Adhesion Molecule-Related/Down-Regulated by Oncogenes (CDON) polypeptide in the preparation of a medicament for treating cancer.

### BRIEF DESCRIPTION OF THE DRAWINGS

FIG. 1 shows that dietary cholesterol triggers FSC proliferation. Hh (green) localizes to Hh producing cells in starved flies (top left, white arrow), and is released and accumulates in FSCs (red triangles) after feeding cholesterol (top right). Bottom: Stem cells proliferate robustly in flies fed yeast, but not yeast extract. Cholesterol addition to yeast extract is sufficient to drive proliferation.

FIG. 2 shows model of nutrient stimulated Hh release in drosophila FSC control. In low nutrient conditions Boi sequesters Hh to producing cells. Introduction of cholesterol leads to steroid-hormone mediated phosphorylation of Boi and Hh release.

FIG. 3 shows S6K-mediated phosphorylation of Boi is required for Hh release. WT Boi in Hh producing cells allows FSC proliferation in fed flies. Mutation of S983 to A abrogates feeding stimulated proliferation. **p<0.00001 vs. fed control.

FIG. 4A shows CDON and SHH are expressed at high levels in pancreatic cancer cell lines. Relative CDON and SHH mRNA levels in immortalized pancreatic ductal epithelial cells compared to three human pancreatic adenocarcinoma cell lines.

FIG. 4B shows that 0.2% of MIA-PaCa cells (blue, DNA) express CDON (red).

FIG. 5A shows CDON and SHH colocalize in Capan-2 cells. Z-stack confocal image of Capan-2 cells showing colocalization (yellow) of CDON (red) and SHH (green) at the apical side of the cells.

FIG. 5B shows CDON (red) and SHH (green) are expressed in a small percentage of tumor cells (blue, DNA) in a genetic PDAC mouse model (K-Ras⁺ p53^{+/-}). CDON⁺ cells (red) are a sub-population of CD44⁺ (green) cells.

FIG. 6 shows SHH is released from starved cells when cholesterol is provided. BxPC3 cells were starved overnight in HBSS. The levels of SHH in the media increases rapidly (within 6 hours) after cholesterol treatment. Overexpression of CDON decreases SHH release. SHH in the media is analyzed by enzyme-linked immunosorbent assay (ELISA).

FIG. 7 shows CDON protein is phosphorylated in starved cells that are stimulated with cholesterol. NIH-3T3 cells were transfected with CDON-GFP and grown in full serum media for 48 hours. Cells were starved for 14 hours in HBSS, and either untreated or treated with cholesterol for 2 hours. Cell lysates were immunoprecipitated with anti-GFP antibody and immunoblotted for CDON or phospho-tyrosine antibody.

FIG. 8 shows SHH release from MIA PaCa-2 cells under varying nutrition conditions. MIA PaCa-2 cells were starved for 18 hours+/−SR1078 and then refed with media and cholesterol alone or media and cholesterol with RORα agonist SR1078.

FIG. 9 shows SHH and CDON are expressed in PanINs and adenocarcinoma in the KPC mouse model. Sections from KPC mouse pancreas showing localization of SHH (green), and CDON (red). Some cells in early PanINs (left) and adenocarcinomas (right) express SHH or CDON. Colocalization is observed in 5-10% of cells (yellow). Nuclei shown in blue.

FIG. 10 shows expression of CDON is high in human PDAC. 13 human pancreas samples embedded in paraffin were stained for CDON (green) and SHH (red). Normal pancreas has no expression of CDON, while PanINs and adenocarcinoma express high levels of CDON in the tumor cells (but not the stroma). Nuclei (blue).

FIG. 11 shows expression of CDON is high in human PDAC. 13 human pancreas samples embedded in paraffin were stained for CDON. Normal pancreas has no expression of CDON, while PanINs and adenocarcinoma express high levels of CDON in the tumor cells. Nuclei (blue).

FIG. 12A shows patient derived xenograft cells release SHH after cholesterol treatment. Cells were starved in HBSS overnight, then treated with cholesterol for 6 hours. SHH levels in the media detected by ELISA.

FIG. 12B shows mutation status of KRas and p53 in all cell types analyzed.

FIG. 13 shows SHH release from pancreatic cancer cells is enhanced when CDON levels are reduced by siRNA. MIA PaCa2 cells were treated with control or CDON siRNA for 48 hours, starved overnight in HBSS, and then fed cholesterol for 6 hours. SHH levels in the media analyzed by SHH ELISA. qRT-PCR showed CDON reduced to 20% of normal levels.

FIG. 14 shows structure of Boi, WT CDON and deletion mutants. CDON is comprised of an extra cellular domain (AA 1-963) that includes a Hh binding domain, a transmembrane domain (TM: AA 964-984), and a cytoplasmic domain (AA: 985-1287). The proposed mutant forms include deletion of the hedgehog binding domain, deletion of the entire cytoplasmic domain, and deletions of three regions of the cytoplasmic domain. All mutant constructs are flanked with an attB recombination sites.

FIG. 15 shows His-tagged CDON is isolated from transfected MIA PaCa-2 cells using Dynabeads® His-Tag Isolation and pulldown beads.

FIG. 16 shows KC (Pdx-Cre/LSL-K-RasG12D) mice develop PanINs by 7-10 months of age, and rarely progress to adenocarcinoma (PDA). KPC mice (Pdx-Cre LSL-KRasG12D TrploxP/loxP) develop PanINs by 2.5 months of age and most progress to adenocarcinoma by 5 months.

FIG. 17 shows sgRNA targeting mouse CDON genomic DNA results in targeted cleavage by Cas9. Four sgRNAs targeting upstream of exon 13, and four targeting downstream of exon 15 were transcribed and resulted in varying efficiencies of cutting of Cas9 cleavage of the DNA in vitro. sgRNA1 is shown as a representative here.

FIG. 18 (Panel A) shows CDON polyclonal antibody immunoblot recognition test. Lanes 1-6: CDON antibodies raised against the N-terminal peptide (SEQ ID NO:2); lanes 7-12: CDON antibodies raised against the C-terminal peptide (SEQ ID NO:3). FIG. 18 (Panel B) shows low exposure of FIG. 18A.

FIG. 19A shows custom anti-CDON antibody generation. Schematic of wild type CDON structure showing domain structure and regions to which custom N- and C-terminal peptides were produced to generate polyclonal antisera.

FIG. 19B shows immunohistochemical detection of CDON protein in tumor tissue from patient derived OC-1 cells with endogenous CDON expression (left panel) and expressing a CDON cDNA construct (right panel) with purified a-CDON antisera.

FIG. 19C shows murine oviduct tissue stained with a-CDON antisera in the absence (left panel) or presence (right panel) of CDON peptide showing successful competition of signal detected by IF.

FIG. 20 (Panel A) shows CDON depletion in OC-1 cells results in significantly decreased xenograft tumor volume. Quantification of tumor volume resulting in mice following implantation of cells transduced with non-targeting gRNA (control) and a targeting gRNA that targets deletion within exon 2 of CDON (ΔEx2) on the left and right flanks respectively (n=7 mice). FIG. 20 (Panel B) shows representative image of tumors isolated from a single mouse. Tumor volume data were analyzed by the non-parametric two-tailed Wilcoxon-Mann-Whitney test (*P=0.0379).

FIG. 21 (Panel A) shows effects of CDON expression on ovarian carcinoma cell sensitivity to carboplatin. Patient-derived OC-1 cells were stably transduced with vector only (control) or a CDON cDNA construct. Following verification of expression of the CDON cDNA construct by WB, cells were analyzed for their sensitivity to carboplatin by CellTiter-Glo® Cell Viability Assay (Promega). FIG. 21 (Panel B) shows effects of CDON expression on ovarian carcinoma cell sensitivity to paclitaxel. Patient-derived OC-1 cells were stably transduced with vector only (control) or a CDON cDNA construct. Following verification of expression of the CDON cDNA construct by WB, cells were analyzed for their sensitivity to paclitaxel by CellTiter-Glo® Cell Viability Assay (Promega).

FIG. 22A shows CDON depletion decreases non-adherent spheroid growth. CRISPR/Cas-9 or siRNA-mediated depletion in OC-1, CaOV-3, and OVCAR-3 cells results in decreased non-adherent spheroid growth. Each individual experiment consisted of a minimum of three technical replicates and each experiment was repeated a minimum of three times. Spheroid formation data was analyzed by nonparametric One-way AN OVA Kruksall-Wallis test with Dunns post-test (*** P<0.0001).

FIG. 22B shows representative images of spheroids in OC-1 and CaOV3 cells with CRISPR/Cas-9-mediated depletion of CDON. Cells transduced with control (non-targeting) and targeted gRNAs for CDON (Aex2 and Aex3).

FIG. 23A shows that CDON regulates OC proliferation and survival. OC-1 cells were transfected with two independent CDON-targeting siRNA constructs and a non-targeting siRNA (control) and cells were assayed for proliferation and apoptosis. Depletion of CDON in OVCAR3 cells results in decreased proliferation as measured by fluorescent DNA incorporation.

FIG. 23B shows that depletion of CDON in OVCAR3 cells results in decreased adherent cell growth as shown via crystal violet staining.

FIG. 23C shows that CDON depletion results in significantly increased cell death (apoptosis) as measured by Annexin V staining.

FIG. 23D shows that CDON depletion results in significantly increased cell death (apoptosis) as measured by western blot detection of cleaved PARP and cleaved caspase-3. One way ANOVA with Dunnetfs post-test (***p<0.005).

FIG. 24A shows CDON protein expression in immortalized (FT190, FT33) and oncogene transformed cells (FT33-TAg-MYC and FT33-TAg-Ras).

FIG. 24B shows lower levels of CDON protein expression in immortalized FTSEC (FT190, FT194, FT246, FT33-TAg) compared to ovarian carcinoma (OC-1, OC-16, OC-29, OC-49, OC-60, and OVCAR-3) cells.

FIG. 24C shows shows that CDON expression is regulated by growth condition. Ovarian carcinoma cells (OVCAR-3 and OC-1) and immortalized and transformed human fallopian tube epithelial cells (FT33-MYC) were grown as adherent 2D monolayer culture (top) and as 3D clusters of cells on non-adherent/low attachment plates. Immunofluoresecent staining of OC cell lines grown as adherent monolayers or as non-adherent multicellular clusters were stained with custom a-CDON antibody (green) and DAPI (blue) showing elevated CDON expression in 3D cultured cells.

FIG. 25A shows CDON fibronectin domain mutant construction. A schematic of CDON mutants constructed to assess the necessity and/or relative importance of each individual fibronectin domain for ovarian carcinoma cell proliferation, survival, cell-to-cell adhesion and tumorigenic potential.

FIG. 25B shows schematic of mutant construction including parental plasmid, and targets of site-directed mutagenesis.

FIG. 26 shows 1600 CDON⁺ MIA-PaCa cells generating a large tumor in NSG mice in 4 weeks.

FIG. 27 (Panel A) shows CDON mRNA expression in OC-PDX tumors. ACTB was used as a normalizing gene and mRNA from MIA-PaCa cells was used as a control for each experiment, with levels set at 100. As an additional control, MIA-PaCa cells transfected with CDON-targeted siRNA showing successful depletion of CDON mRNA. Bars labeled with asterisks indicate PDX models established from ascites, all others from solid tumor. FIG. 27 (Panel B) shows CDON mRNA expression in primary OC specimens. FIG. 27 (Panel C) shows CDON mRNA expression in established and patient-derived OC cell lines. Bars labeled with asterisks indicate PDX models established from ascites, all others from solid tumor. FIG. 27 (Panel D) shows tumor tissue from OC-PDX models labeled with stars in (FIG. 27, Panel A) was FACS sorted to determine the % CDON⁺ cells in the tumor.

FIG. 28 (Panel A) shows patient-derived OC-cells grown in 2D monolayer or suspension and stained with Aldefluor or CDON antibodies. FIG. 28 (Panel B) shows patient-derived OC-20 cells grown in 2D monolayer or suspension and stained with Aldefluor or CDON antibodies. FIG. 28 (Panel C) shows western blot detection of CDON in OC-1 and OC-20 cells grown in monolayer (M) or suspension (S). Scale bars=50 μm.

FIG. 29A shows PDX model OC-1 grown by subcutaneous injection as a solid tumor (top panels) or by intraperitoneal injection as diffuse ascites (bottom panels), tumors were formalin fixed paraffin embedded and stained with antibodies recognizing WT1 (Wilms tumor antigen, a characteristic marker of high grade serous carcinomas to distinguish tumor from stromal cells) and CDON.

FIG. 29B shows flow cytometry analysis of tumors disaggregated to a single cell suspension with anti-CDON antibodies showing >18-fold elevation of CDON⁺ cells in ascites compared to solid tumor.

FIG. 30 (Panel A) shows immunoblots of E- and N-cadherins in OC PDXs. FIG. 30 (Panel B) shows UWB.289 cells grown in 2D monolayer or suspension and stained with Ecadherin or CDON antibodies. FIG. 30 (Panel C) shows OC-PDX cells (OC-1 and OC-16) grown as organoids and stained for CDON and E-cadherin. Scale bars=75 (A) or 25 μm (B).

FIG. 31 (Panel A) shows H&E and IHC stained sections for detection of cytokeratin, p53 and PAX8 in a metastatic patient tumor and the corresponding PDX tumors (P0 and P1 grafts) in mice having consistent histology and biomarker expression. FIG. 31 (Panel B) shows aCGH of DNA isolated from matched patient and PDX tumor (OC-1) demonstrating extensive genomic alterations in the patient tumor are maintained in the PDX tumor. FIG. 31 (Panel C) shows ascites harvested from a P1 mouse and injected i.p. in recipient P2 SCID mice equal volumes of tumor cells. The recipient mice were treated weekly with vehicle or paclitaxel (5 (n=5/group) for four weeks. At necropsy, tumor nodules were enumerated and the total number of viable cells present in the ascites determined using an automated cell counter. Data were analyzed by the Mann-Whitney test (*p<0.05).

FIG. 32 (Panel A) shows that selection for ALDH1⁺ and CD133⁺ positivity in tumor from PDX OC-38 isolates an infrequent sub-population (7%) of cells. FIG. 32 (Panel B) shows that ALDH1+ and CD133⁺ positive cells display increased spheroid forming capacity. FIG. 32 (Panel C) depcits representative images showing that ALDH1⁺ and CD133⁺ positive cells display increased spheroid forming capacity. FIG. 32 (Panel D) shows that ALDH1⁺ and CD133⁺ positive cells exhibit low sensitivity to paclitaxel, but high sensitivity to treatment with the HSP90 inhibitor ganetespib.

FIG. 33 shows expression of CDON and Hh pathway genes is elevated in cells grown in suspension.

FIG. 34 shows OC-1 and OC-16 cells with CRISPR/Cas9-mediated depletion of CDON show alterations in several signaling, stem and EMT proteins by western blot analysis. Overexpression of His-CDON in FTSEC cell line FT246 results in opposite alterations in several signaling, stem and EMT proteins.

FIG. 35 (Panel A) shows overexpression of His-CDON in FTSEC cell FT246 cells results in increased number of spheres across sizes (Sphere number and size determined using ImageJ). FIG. 35 (Panel B) shows average fold increase in sphere number in FT246-His-CDON cells compared to control. FIG. 35 (Panel C) shows increased tumorsphere forming efficiency calculated in OC-1-His-CDON cells compared to control. Data shown are mean values from three independent experiments with 16-32 replicates for each condition tested in each experiment. Student's t-test and values <0.05 are considered significant.

FIG. 36 shows CRISPR/Cas9-mediated depletion of CDON in ovarian carcinoma (OC-1) cells.

FIG. 37A shows that CRISPR/Cas9-mediated depletion of CDON in OC-1 results in decreased size of spheres (Sphere number and size determined using ImageJ).

FIG. 37B shows that average fold decrease in sphere size in CDON depleted OC-1 cells compared to parental control.

FIG. 37C shows decreased tumorsphere forming efficiency calculated in OC-1 CDON depleted cells compared to control. Data shown are mean values from three independent experiments with 16-32 replicates for each condition tested in each experiment. Student's t-test and values <0.05 are considered significant.

FIG. 38A shows ELISA screening analysis of supernatants collected from mouse 5 splenocyte fusions to an immobilized OV-conjugated CDON peptide.

FIG. 38B shows the selected hits that were selected for expansion and further testing.

FIG. 39A shows secondary ELISA absorbance analysis of the 51 hits.

FIG. 39B shows the clone map identifying the highest scoring by ELISA.

FIG. 40 shows the effects of clone supernatants on OVCAR-3 cell morphology and viability.

### DESCRIPTION OF EMBODIMENTS

The terminology used herein is for the purpose of describing particular embodiments only and is not intended to be limiting. Various terms relating to aspects of disclosure are used throughout the specification and claims. Such terms are to be given their ordinary meaning in the art, unless otherwise indicated. Other specifically defined terms are to be construed in a manner consistent with the definition provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, “about” means the numerical value can vary by ±10% and remain within the scope of the disclosed embodiments.

As used herein, the terms “subject” and “patient” are used interchangeably. A subject may include any animal, including mammals. Mammals include, without limitation, farm animals (e.g., horse, cow, pig), companion animals (e.g., dog, cat), laboratory animals (e.g., mouse, rat, rabbits), and non-human primates. In some embodiments, the subject is a human.

As used herein, the term “epitope” refers to a portion of a sequence of contiguous or non-contiguous amino acids (in an antigen) which is recognized by and bound by a detection agent such as an antibody, or antigen-binding fragment thereof. In some embodiments, the epitope is a linear epitope on a polypeptide which typically includes 3 to 10 or 6 to 10 contiguous amino acids that are recognized and bound by a detection agent. A conformational epitope includes non-contiguous amino acids. The detection agent, such as an antibody or antigen-binding fragment thereof, recognizes the 3-dimensional structure.

As used herein, the term “antigen” refers to any substance capable, under appropriate conditions, of inducing a specific immune response and reacting with the products of that response (e.g., specific antibody and/or specifically sensitized T lymphocytes). The present disclosure provides human antibodies to human CDON antigens. The antibodies or antigen-binding fragments thereof disclosed herein may mediate molecular and/or cellular effector functions such as complement-mediated lysis, phagocytosis, or killing by natural killer cells or may block or antagonize signals transduced by cell surface receptors. The antibodies may also bind to an epitope on a human receptor to inhibit the receptor from interacting with a ligand or co-receptor.

As used herein, the term “antibody” refers to the structure that constitutes the natural biological form of an antibody. In most mammals, including humans, and mice, this form is a tetramer and consists of two identical pairs of two immunoglobulin chains, each pair having one light and one heavy chain, each light chain comprising immunoglobulin domains V_{L} and C_{L}, and each heavy chain comprising immunoglobulin domains V_{H}, Cγ1, Cγ2, and Cγ3. In each pair, the light and heavy chain variable regions (V_{L} and V_{H}) are together responsible for binding to an antigen, and the constant regions (CL, Cγ1, Cγ2, and Cγ3, particularly Cγ2, and Cγ3) are responsible for antibody effector functions.

Depending on the amino acid sequence of the constant domain of their heavy chains, intact antibodies can be assigned to different “classes.” There are five-major classes of intact antibodies: IgA, IgD, IgE, IgG, and IgM, and several of these may be further divided into “subclasses” (i.e., isotypes), such as IgG1, IgG2, IgG3, IgG4, IgA, and IgA2. The heavy chain constant domains that correspond to the different classes of antibodies are termed alpha, delta, epsilon, gamma, and mu, respectively.

An ““isolated” polypeptide is a polypeptide that is found in a condition other than its native environment, such as apart from blood and animal tissue. In some embodiments, the isolated polypeptide is substantially free of other polypeptides, particularly other polypeptides of animal origin. In some embodiments, the polypeptides are present in a highly purified form, i.e., greater than 95% pure or greater than 99% pure. When used in this context, the term “isolated” does not exclude the presence of the same polypeptide in alternative physical forms, such as dimers or alternatively glycosylated or derivatized forms.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In some embodiments, the antibody will be purified: 1) to greater than 95% by weight or to greater than 99% by weight of antibody as determined by the Lowry method, 2) to a degree sufficient to obtain at least 15 residues of N-terminal or internal amino acid sequence by use of a spinning cup sequenator, or 3) to homogeneity by SDS-PAGE under reducing or nonreducing conditions using Coomassie blue or silver stain. An isolated antibody includes the antibody in situ within recombinant cells since at least one component of the antibody's natural environment will not be present. Ordinarily, however, an isolated antibody will be prepared by at least one purification step.

As used herein, the terms “K_{assoc}” or “Kₐ” refers to the association rate of a particular antibody-antigen interaction, whereas the terms “K_{dis}” or “K_{d},” refers to the dissociation rate of a particular antibody-antigen interaction. As used herein, the term “K_{D}” refers to the dissociation constant, which is obtained from the ratio of K_{d} to Kₐ (i.e., K_{d}/Kₐ) and is expressed as a molar concentration (M). K_{D} values for antibodies can be determined using methods well established in the art. In some embodiments, the antibody or antigen-binding fragment thereof binds its target with a Kd of about 0.1 nM.

In some embodiments, the terms “binds” or “binding’ or grammatical equivalents thereof, refer to the compositions having an affinity for each other. As used herein, “specific binding” refers to preferential binding of an antibody to a specified antigen relative to other non-specified antigens. The phrase “specifically (or selectively) binds” to an antibody refers to a binding reaction that is determinative of the presence of the protein in a heterogeneous population of proteins and other biologies. Typically, the antibody binds with a dissociation constant (K_{D}) of about 1×10⁻⁷ M or less, about 1×10⁻⁸ M or less, about 1×10⁻⁹ M or less, about 1×10⁻¹⁰ M or less, about 1×10⁻¹¹ M or less, or about 1×10⁻¹² M or less, and binds to the specified antigen with an affinity that is at least two-fold greater than its affinity for binding to a non-specific antigen (e.g., BSA, KLH, casein, etc.) other than the specified antigen or a closely-related antigen. Specific binding can be measured by, for example, determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. In some embodiments, such terms refer to binding where a molecule binds to a particular polypeptide or epitope on a particular polypeptide without substantially binding to any other polypeptide or polypeptide epitope. The phrases “an antibody recognizing an antigen” and “an antibody specific for an antigen” are used interchangeably herein with the term “an antibody that binds specifically to an antigen.” A predetermined antigen is an antigen that is chosen prior to the selection of an antibody that binds to that antigen.

As used herein, the term “polyclonal antibody” refers to a mixture of antibodies which are genetically different due to, for example, production by different plasma cells and which recognize a different epitope of the same antigen.

As used herein, the term “monoclonal antibody” refers to an antibody from a population of substantially homogeneous antibodies, i.e., the individual antibodies comprising the population are identical and/or bind the same epitope(s), except for possible variants that may arise during production of the monoclonal antibody, such variants generally being present in minor amounts. Such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones or recombinant DNA clones. It should be understood that the selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also considered herein to be a monoclonal antibody. In contrast to a polyclonal antibody preparation, which typically includes different antibodies directed against different determinants (epitopes), each monoclonal antibody of a monoclonal antibody preparation is directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure can be made by a variety of techniques, including, for example, the hybridoma method (e.g., Kohler et al., Nature, 1975, 256, 495; Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N.Y., 1981)), recombinant DNA methods (see, e.g., U.S. Pat. No. 4,816,567), phage display technologies (see, e.g., Clackson et al., Nature, 1991, 352, 624-628; Marks et al., J. Mol. Biol., 1991, 222, 581-597; Sidhu et al., J. Mol. Biol., 2004, 338, 299-310; Lee et al., J. Mol. Biol., 2004, 340, 1073-1093; Fellouse, Proc. Nat. Acad. Sci. USA, 2004, 101, 12467-12472; and Lee et al., J. Immunol. Methods, 2004, 284, 119-132), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA, 1993, 90, 2551; Jakobovits et al., Nature, 1993, 362, 255-258; Bruggemann et al., Year in Immuno., 1993, 7, 33; U.S. Pat. Nos. 5,545,806; 5,569,825; 5,591,669; 5,545,807; WO 1997/17852; U.S. Pat. Nos. 5,545,807; 5,545,806; 5,569,825; 5,625,126; 5,633,425; and U.S. Pat. No. 5,661,016; Marks et al., Bio/Technology, 1992, 10, 779-783; Lonberg et al., Nature, 1994, 368, 856-859; Morrison, Nature, 1994, 368, 812-813; Fishwild et al., Nature Biotechnology, 1996, 14, 845-851; Neuberger, Nature Biotechnology, 1996, 14, 826; and Lonberg and Huszar, Intern. Rev. Immunol., 1995, 13, 65-93). Monoclonal antibodies useful with the present disclosure can also be prepared using a wide variety of non-hybridoma techniques known in the art including the use of recombinant, and phage display technologies, or a combination thereof.

As used herein, the term “chimeric antibody” refers to an antibody that has a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. Nos. 5,807,715; 4,816,567; and 4,816,397; and Morrison et al., Proc. Natl. Acad. Sci. USA, 1984, 81, 6851-6855; Morrison, Science, 1985, 229, 1202-1207; Oi et al., BioTechniques, 1986, 4, 214-221; and Gillies et al., J. Immunol. Methods, 1985, 125, 191-202). A humanized antibody is a type of a chimeric antibody.

“Humanized” forms of non-human (e.g., murine) antibodies are chimeric immunoglobulins that contain minimal sequences derived from non-human immunoglobulins. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., Nature, 1988, 332, 323-327; U.S. Pat. Nos. 5,530,101; 5,585,089; 5,693,761; 5,693,762; and 6,180,370; EP239400; PCT publication WO 91/09967; U.S. Pat. No. 5,225,539; EP592106; EP519596; Padlan, Mol. Immunol., 1991, 28, 489-498; Studnicka et al., Prot. Eng., 1994, 7, 805-814; Roguska et al., Proc. Natl. Acad. Sci., 1994, 91, 969-973; and U.S. Pat. No. 5,565,332.

“Human antibodies” include antibodies having the amino acid sequence of a human immunoglobulin and include antibodies isolated from human immunoglobulin libraries or from animals transgenic for one or more human immunoglobulins and that do not express endogenous immunoglobulins. Human antibodies can be made by a variety of methods known in the art including phage display methods using antibody libraries derived from human immunoglobulin sequences. See U.S. Pat. Nos. 4,444,887 and 4,716,111; and PCT publications WO 98/46645; WO 98/50433; WO 98/24893; WO 98/16654, WO 96/34096; ‘WO 96/33735; and WO 91/10741. Human antibodies can also be produced using transgenic mice which are incapable of expressing functional endogenous immunoglobulins but which can express human immunoglobulin genes. See, e.g., PCT publications WO 98/24893; WO 92/01047; WO 96/34096; WO 96/33735; U.S. Pat. Nos. 5,413,923; 5,625,126, 5,633,425; 5,569,825; 5,661,016; 5,545,806; 5,814,318; 5,885,793, 5,916,771; and 5,939,598. Fully human antibodies that recognize a selected epitope can be generated using a technique referred to as “graded selection.” In this approach, a selected non-human monoclonal antibody, e.g., a mouse antibody, is used to guide the selection of a completely human antibody recognizing the same epitope (see, Jespers et al., Biotechnology. 1988, 12, 899-903).

As used herein, the term “recombinant antibody” includes all antibodies of the disclosure that are prepared, expressed, created, or isolated by recombinant means, such as antibodies isolated from an one animal (e.g., a mouse) that is transgenic for another animal's (e.g. a dog) immunoglobulin genes (described further below); antibodies expressed using a recombinant expression vector transfected into a host cell, antibodies isolated from a recombinant, combinatorial antibody library, or antibodies prepared, expressed, created or isolated by any other means that involves splicing of immunoglobulin gene sequences to other DNA sequences. Such recombinant antibodies have variable and constant regions (if present) derived from a particular animal's germline immunoglobulin sequences. Such antibodies can, however, be subjected to in vitro mutagenesis (or, when an animal transgenic for another species Ig sequences is used, in vivo somatic mutagenesis) and, thus, the amino acid sequences of the V_{H} and V_{L} regions of the recombinant antibodies are sequences that, while derived from and related to e.g. human germline V_{H} and V_{L} sequences, may not naturally exist within the human antibody germline repertoire in vivo. The present disclosure also provides for antigen-binding fragments of anti-CDON antibodies.

As used herein, the term “antigen-binding fragment” refers to functional antibody fragments, such as Fab, a scFv-Fc bivalent molecule, F(ab′)₂, and F_{V} that are capable of specifically interacting with a desired target. In some embodiments, the antigen-binding fragments comprise: 1) Fab, the fragment which contains a monovalent antigen-binding fragment of an antibody molecule, which can be produced by digestion of whole antibody with the enzyme papain to yield an intact light chain and a portion of one heavy chain; 2) Fab′, the fragment of an antibody molecule that can be obtained by treating whole antibody with pepsin, followed by reduction, to yield an intact light chain and a portion of the heavy chain; two Fab′ fragments are obtained per antibody molecule; 3) (Fab′)₂, the fragment of the antibody that can be obtained by treating whole antibody with the enzyme pepsin without subsequent reduction; F(ab′)₂ is a dimer of two Fab′ fragments held together by two disulfide bonds; 4) F_{V}, a genetically engineered fragment containing the variable region of the light chain and the variable region of the heavy chain expressed as two chains; and 5) single chain antibody (“SCA”), a genetically engineered molecule containing the variable region of the light chain and the variable region of the heavy chain, linked by a suitable polypeptide linker as a genetically fused single chain molecule.

scFv-Fc can be produced by fusing single-chain Fv (scFv) with a hinge region from an immunoglobulin (Ig) such as an IgG, and Fc regions.

A Fab fragment contains the constant domain of the light chain and the first constant domain (C_{H1}) of the heavy chain. Fab′ fragments differ from Fab fragments by the addition of a few residues at the carboxyl terminus of the heavy chain C_{H1} domain including one or more cysteines from the antibody hinge region. F(ab′) fragments are produced by cleavage of the disulfide bond at the hinge cysteines of the F(ab′)₂ pepsin digestion product. Additional chemical couplings of antibody fragments are known to those of ordinary skill in the art. Fab and F(ab′)₂fragments lack the Fc fragment of an intact antibody, clear more rapidly from the circulation of animals, and may have less non-specific tissue binding than an intact antibody (see, e.g., Wahl et al., J. Nucl. Med., 1983, 24, 316).

An “Fv” fragment is the minimum fragment of an antibody that contains a complete target recognition and binding site. This region consists of a dimer of one heavy and one light chain variable domain in a tight, non-covalent association (V_{H}-V_{L} dimer). It is in this configuration that the three CDRS of each variable domain interact to define a target binding site on the surface of the V_{H}-V_{L} dimer. Often, the six CDRs confer target binding specificity to the antibody. However, in some instances even a single variable domain (or half of an Fv comprising only three CDRs specific for a target) can have the ability to recognize and bind target, although at a lower affinity than the entire binding site.

“Single-chain Fv” or “scFv” antibody binding fragments comprise the V_{H} and V_{L} domains of an antibody, where these domains are present in a single polypeptide chain. Generally, the Fv polypeptide further comprises a polypeptide linker between the V_{H} and V_{L} domains which enables the scFv to form the desired structure for target binding.

“Single domain antibodies” are composed of a single V_{H} or V_{L} domains which exhibit sufficient affinity to CDON. In some embodiments, the single domain antibody is a camelized antibody (see, e.g., Riechmann, J. Immunolog. Methods, 1999, 231, 25-3 8).

As used herein, the term “CDR” or “complementarity determining region” refers to amino acid residues comprising non-contiguous antigen combining sites found within the variable region of both heavy and light chain polypeptides. In some embodiments, the term “CDR” will comprise regions as described by Kabat et al., J. Biol. Chem., 1977, 252, 6609-6616 and Kabat et al., Sequences of protein of immunological interest. (1991), and Chothia and Lesk, Mol. Biol., 1987, 196, 901-917 and MacCallum et al., Mol. Biol., 1996, 262, 732-745. The amino acids of the CDRs of the variable domains were initially defined by Kabat, based on sequence variability, to consist of amino acid residues 31-35B (HI), 50-65 (H2), and 95-102 (H3) in the human heavy chain variable domain (V_{H}) and amino acid residues 24-34 (LI), 50-56 (L2), and 89-97 (L3) in the human light chain variable domain (V_{L}), using Kabaks numbering system for amino acid residues of an antibody. See Kabat et al., sequences of proteins of immunological interest, US Dept. Health and Human Services, NIH, USA (5th ed. 1991). Chothia and Lesk, J. Mol. Biol., 1987, 196, 901-917 presented another definition of the CDRs based on residues that included in the three-dimensional structural loops of the variable domain regions, which were found to be important in antigen binding activity. Chothia et al. defined the CDRs as consisting of amino acid residues 26-32 (H1), 52-56 (H2), and 95-102 (H3) in the human heavy chain variable domain (V_{H}), and amino acid residues 24-34 (LI), 50-56 (L2), and 89-97 (L3) in the human light chain variable domain (V_{L}). Combining the CDR definitions of Kabat and Chothia, the CDRs consist of amino acid residues 26-35B (H1), 50-65 (H2), and 95-102 (H3) in human V_{H} and amino acid residues 24-34 (LI), 50-56 (L2), and 89-97 (L3) in human V_{L}, based on Kabaks numbering system.

The anti-CDON antibodies of the disclosure can be primatized. As used herein, the term “primatized antibody” refers to an antibody comprising monkey variable regions and human constant regions. Methods for producing primatized antibodies are known in the art. See e.g., U.S. Pat. Nos. 5,658,570; 5,681,722; and 5,693,780.

The present disclosure provides antibodies, and antigen-binding fragments thereof, that specifically bind particular regions of CDON polypeptide, and inhibit its function. The human CDON polypeptide has a length of 1287 amino acids. The amino acid sequence of human CDON is:

(SEQ ID NO: 1)
MHPDLGPLCTLLYVTLTILCSSVSSDLAPYFTSEPLSAVQKLGGPVV
LHCSAQPVTTRISWLHNGKTLDGNLEHVKIHQGTLTILSLNSSLLGYYQCLANNSIGA
IVSGPATVSVAVLGDFGSSTKHVITAEEKSAGFIGCRVPESNPKAEVRYKIRGKWLEH
STENYLILPSGNLQILNVSLEDKGSYKCAAYNPVTHQLKVEPIGRKLLVSRPSSDDVH
ILHPTHSQALAVLSRSPVTLECVVSGVPAPQVYWLKDGQDIAPGSNWRRLYSHLATD
SVDPADSGNYSCMAGNKSGDVKYVTYMVNVLEHASISKGLQDQIVSLGATVHFTC
DVHGNPAPNCTWFHNAQPIHPSARHLTAGNGLKISGVTVEDVGMYQCVADNGIGF
MHSTGRLEIENDGGFKPVIITAPVSAKVADGDFVTLSCNASGLPVPVIRWYDSHGLIT
SHPSQVLRSKSRKSQLSRPEGLNLEPVYFVLSQAGASSLHIQAVTQEHAGKYICEAAN
EHGTTQAEASLMVVPFETNTKAETVTLPDAAQNDDRSKRDGSETGLLSSFPVKVHPS
AVESAPEKNASGISVPDAPIILSPPQTHTPDTYNLVWRAGKDGGLPINAYFVKYRKLD
DGVGMLGSWHTVRVPGSENELHLAELEPSSLYEVLMVARSAAGEGQPAMLTFRTSK
EKTASSKNTQASSPPVGIPKYPVVSEAANNNFGVVLTDSSRHSGVPEAPDRPTISTAS
ETSVYVTWIPRANGGSPITAFKVEYKRMRTSNWLVAAEDIPPSKLSVEVRSLEPGSTY
KFRVIAINHYGESFRSSASRPYQVVGFPNRFSSRPITGPHIAYTEAVSDTQIMLKWTYI
PSSNNNTPIQGFYIYYRPTDSDNDSDYKRDVVEGSKQWHMIGHLQPETSYDIKMQCF
NEGGESEFSNVMICETKVKRVPGASEYPVKDLSTPPNSLGSGGNVGPATSPARSSDM
LYLIVGCVLGVMVLILMVFIAMCLWKNRQQNTIQKYDPPGYLYQGSDMNGQMVDY
TTLSGASQINGNVHGGFLTNGGLSSGYSHLHHKVPNAVNGIVNGSLNGGLYSGHSNS
LTRTHVDFEHPHHLVNGGGMYTAVPQIDPLECVNCRNCRNNNRCFTKTNSTFSSSPP
PVVPVVAPYPQDGLEMKPLSHVKVPVCLTSAVPDCGQLPEESVKDNVEPVPTQRTC
CQDIVNDVSSDGSEDPAEFSRGQEGMINLRIPDHLQLAKSCVWEGDSCAHSETEINIV
SWNALILPPVPEGCAEKTMWSPPGIPLDSPTEVLQQPRET.

In some embodiments, the antibody, or antigen-binding fragment thereof, does not bind to a region of the CDON polypeptide consisting of positions corresponding to positions 456 to 598, to positions 480 to 560, to positions 1155 to 1264, to positions 511 to 560, or to positions 990 to 1002 according to SEQ ID NO: 1.

In some embodiments, the particular regions of the CDON polypeptide to which the antibodies, and antigen-binding fragments thereof, bind consist of 14 to 20 amino acids, 15 to 20 amino acids, 16 to 19 amino acids, or 17 to 18 amino acids. In some embodiments, the particular regions of the CDON polypeptide to which the antibodies, and antigen-binding fragments thereof, bind consist of 17 or 18 amino acids. In some embodiments, the particular regions of the CDON polypeptide to which the antibodies, and antigen-binding fragments thereof, bind consist of 14 or 15 amino acids. In some embodiments, the particular regions of the CDON polypeptide to which the antibodies, and antigen-binding fragments thereof, bind consist of 17 amino acids. In some embodiments, the particular regions of the CDON polypeptide to which the antibodies, and antigen-binding fragments thereof, bind consist of 18 amino acids.

In some embodiments, the antibody, or antigen-binding fragment thereof, specifically binds: a) a CDON polypeptide consisting of amino acids at positions corresponding to positions 1 to 200 according to SEQ ID NO: 1; or b) a CDON polypeptide consisting of amino acids at positions corresponding to positions 1000 to 1287 according to SEQ ID NO: 1.

In some embodiments, the antibody, or antigen-binding fragment thereof, specifically binds: a) a CDON polypeptide consisting of amino acids at positions corresponding to positions 100 to 200 according to SEQ ID NO: 1; or b) a CDON polypeptide consisting of amino acids at positions corresponding to positions 1200 to 1287 according to SEQ ID NO: 1.

In some embodiments, the antibody, or antigen-binding fragment thereof, specifically binds: a) a CDON polypeptide consisting of amino acids at positions corresponding to positions 140 to 170 according to SEQ ID NO: 1; or b) a CDON polypeptide consisting of amino acids at positions corresponding to positions 1250 to 1287 according to SEQ ID NO: 1.

In some embodiments, the antibody, or antigen-binding fragment thereof, specifically binds: a CDON peptide consisting of the amino acid sequence RVPESNPKAEVRYKIRGK (SEQ ID NO:2), a CDON peptide consisting of the amino acid sequence GIPLDSPTEVLQQPRET (SEQ ID NO:3), a CDON peptide consisting of the amino acid sequence VLGDFGSSTKHVITAEE (SEQ ID NO:4), or a CDON peptide consisting of the amino acids sequence KIRGKWLEHSTENY (SEQ ID NO:5).

In some embodiments, the antibody, or antigen-binding fragment thereof, specifically binds a CDON peptide consisting of the amino acid sequence RVPESNPKAEVRYKIRGK (SEQ ID NO:2). In some embodiments, the antibody, or antigen-binding fragment thereof, specifically binds a CDON peptide consisting of the amino acid sequence GIPLDSPTEVLQQPRET (SEQ ID NO:3). In some embodiments, the antibody, or antigen-binding fragment thereof, specifically binds a CDON peptide consisting of the amino acid sequence VLGDFGSSTKHVITAEE (SEQ ID NO:4). In some embodiments, the antibody, or antigen-binding fragment thereof, specifically binds a CDON peptide consisting of the amino acid sequence KIRGKWLEHSTENY (SEQ ID NO:5)

In some embodiments, the anti-CDON antibody, or antigen-binding fragment thereof, specifically binds to an epitope within the N-terminus of CDON. In some embodiments, the anti-CDON antibody, or antigen-binding fragment thereof, specifically binds to an epitope within residues 1 to 200, 50 to 200, 100 to 200, 125 to 175, or 140 to 170 according to SEQ ID NO: 1. In some embodiments, the anti-CDON antibody, or antigen-binding fragment thereof, specifically binds to an epitope within residues 50 to 200, 100 to 200, 125 to 175, or 140 to 170 according to SEQ ID NO: 1. In some embodiments, the anti-CDON antibody, or antigen-binding fragment thereof, specifically binds to an epitope within residues 100 to 200, 125 to 175, or 140 to 170 according to SEQ ID NO: 1. In some embodiments, the anti-CDON antibody, or antigen-binding fragment thereof, specifically binds to an epitope within residues 125 to 175, or 140 to 170 according to SEQ ID NO: 1. In some embodiments, the anti-CDON antibody, or antigen-binding fragment thereof, specifically binds to an epitope within residues 140 to 170 according to SEQ ID NO: 1. In some embodiments, the anti-CDON antibody, or antigen-binding fragment thereof, specifically binds to an epitope within residues 142 to 159 according to SEQ ID NO: 1. In some embodiments, the anti-CDON antibody, or antigen-binding fragment thereof, specifically binds to an epitope within residues 117 to 133 according to SEQ ID NO: 1. In some embodiments, the anti-CDON antibody, or antigen-binding fragment thereof, specifically binds to an epitope within residues 155-168 according to SEQ ID NO: 1.

In some embodiments, the anti-CDON antibody, or antigen-binding fragment thereof, specifically binds to an epitope within the C-terminus of CDON. In some embodiments, the anti-CDON antibody, or antigen-binding fragment thereof, specifically binds to an epitope within residues 1000 to 1287, 1100 to 1287, 1200 to 1287, 1225 to 1287, or 1250 to 1287 according to SEQ ID NO: 1. In some embodiments, the anti-CDON antibody, or antigen-binding fragment thereof, specifically binds to an epitope within residues 1100 to 1287, 1200 to 1287, 1225 to 1287, or 1250 to 1287 according to SEQ ID NO:1. In some embodiments, the anti-CDON antibody, or antigen-binding fragment thereof, specifically binds to an epitope within residues 1200 to 1287, 1225 to 1287, or 1250 to 1287 according to SEQ ID NO: 1. In some embodiments, the anti-CDON antibody, or antigen-binding fragment thereof, specifically binds to an epitope within residues 1225 to 1287 or 1250 to 1287 according to SEQ ID NO: 1. In some embodiments, the anti-CDON antibody, or antigen-binding fragment thereof, specifically binds to an epitope within residues 1250 to 1287 according to SEQ ID NO: 1. In some embodiments, the anti-CDON antibody, or antigen-binding fragment thereof, specifically binds to an epitope within residues 1271 to 1287 according to SEQ ID NO:1.

The anti-CDON antibodies, or antigen-binding fragments thereof, in the present disclosure can be polyclonal, monoclonal, genetically engineered, and/or otherwise modified in nature, including but not limited to chimeric antibodies, humanized antibodies, human antibodies, recombinant antibodies, single chain antibodies, etc. In some embodiments, the antibodies comprise all or a portion of a constant region of an antibody. In some embodiments, the constant region is an isotype selected from: IgA (e.g., IgA₁ or IgA₂), IgD, IgE, IgG (e.g., IgG₁, IgG₂, IgG₃ or IgG₄), and IgM. As used herein, the ““‘constant region” of an antibody includes the natural constant region, allotypes or natural variants, such as D356E and L358M, or A431G in human IgG₁. See, e.g., Jefferis and Lefranc, MAbs, 2009, 1, 332-338.

The light chain of an anti-CDON antibody, or antigen-binding fragment thereof, can be a kappa (κ) light chain or a lambda (λ) light chain. A λ light chain can be any one of the known subtypes, e.g., λ₁, λ₂, λ₃, or λ₄. In some embodiments, the anti-CDON antibody comprises a kappa (κ) light chain.

In some embodiments, the antibody is a polyclonal antibody. In some embodiments, the antibody is a monoclonal antibody. In some embodiments, the antigen-binding fragment is a single chain Fv (scFv), a single domain fragment, a diabody, a tandem scFv, a scFv-Fc bivalent molecule, an Fab, Fab′, Fv, or F(ab′)₂.

In some embodiments, the anti-CDON antibodies are bispecific antibodies. Bispecific antibodies are monoclonal, often human or humanized, antibodies that have binding specificities for at least two different antigens. In the present disclosure, one of the binding specificities can be directed towards CDON, the other can be for any other antigen, e.g., for a cell-surface protein, receptor, receptor subunit, tissue-specific antigen, virally derived protein, virally encoded envelope protein, bacterially derived protein, or bacterial surface protein, etc.

In some embodiments, the antibody or antigen-binding fragment thereof provided herein comprises a modification. In some embodiments, the modification minimizes conformational changes during the shift from displayed to secreted forms of the antibody or antigen-binding fragment. It is to be understood by a skilled artisan that the modification can be a modification known in the art to impart a functional property that would not otherwise be present if it were not for the presence of the modification. The present disclosure encompasses antibodies which are differentially modified during or after translation, e.g., by pegylation, glycosylation, acetylation, phosphorylation, amidation, derivatization by known protecting/blocking groups, proteolytic cleavage, linkage to an antibody molecule, another protein or other cellular ligand, etc. Any of numerous chemical modifications may be carried out by known techniques, including but not limited, to specific chemical cleavage by cyanogen bromide, trypsin, chymotrypsin, papain, V8 protease, NaBH4, acetylation, formylation, oxidation, reduction, metabolic synthesis in the presence of tunicamycin, etc. Additionally, the derivative can contain one or more non-natural amino acids, e.g., using ambrx technology (See, e.g., Wolfson, Chem. Biol., 2006, 13, 1011-1012).

Additional post-translational modifications include, for example, e.g., N-linked or O-linked carbohydrate chains, processing of N-terminal or C-terminal ends, attachment of chemical moieties to the amino acid backbone, chemical modifications of N-linked or O-linked carbohydrate chains, and addition or deletion of an N-terminal methionine residue as a result of procaryotic host cell expression.

In some embodiments, the anti-CDON antibodies are derivatized through glycosylation. Common biantennary complexes can be composed of a core structure having two N-acetylglucosamine (GlcNAc), three mannose, and two GlcNAc residues that are β-1,2 linked to α-6 mannose and α-3 mannose to form two antennae. One or more fucose (Fuc), galactose (Gal), high mannose glycans Man-5 or Man-9, bisecting GlcNAc, and sialic acid including N-acetylneuraminic acid (NANA) or N-glycolylneuraminic acid (NGNA) residues may be attached to the core. N-linked glycoforms may include G0 (protein having a core biantennary glycosylation structure), G0F (fucosylated G0), GOF GlcNAc, G1 (protein having a core glycosylation structure with one galactose residue), GIF (fucosylated G1), G2 (protein having a core glycosylation structure with two galactose residues), and/or G2F (fucosylated G2). In some embodiments, an anti-CDON antibody has a GOF glycan.

In some embodiments, the modification is an N-terminus modification. In some embodiments, the modification is a C-terminal modification. In some embodiments, the modification is an N-terminus biotinylation. In some embodiments, the modification is a C-terminus biotinylation. In some embodiments, the secretable form of the antibody or antigen-binding fragment comprises an N-terminal modification that allows binding to an Ig hinge region. In some embodiments, the Ig hinge region is from an IgA hinge region. In some embodiments, the secretable form of the antibody or antigen-binding fragment comprises an N-terminal modification that allows binding to an enzymatically biotinylatable site. In some embodiments, the secretable form of the antibody or antigen-binding fragment comprises an C-terminal modification that allows binding to an enzymatically biotinylatable site. In some embodiments, biotinylation of the site functionilizes the site to bind to any surface coated with streptavidin, avidin, avidin-derived moieties, or a secondary reagent. In some embodiments, the secondary reagent is a protein, a peptide, a carbohydrate, or a glycoprotein.

In some embodiments, the anti-CDON antibodies, or antigen-binding fragments thereof, can be modified for increased expression in heterologous hosts. In some embodiments, the anti-CDON antibodies, or antigen-binding fragments thereof, can be modified for secretion from heterologous host cells. In some embodiments, the anti-CDON antibodies, or antigen-binding fragments thereof, can be modified for increased expression in bacteria, such as E. coli. In some embodiments, the anti-CDON antibodies, or antigen-binding fragments thereof, can be modified for increased expression in yeast (see, Kieke et al., Proc. Nat'l Acad. Sci. USA, 1999, 96, 5651-5656). In some embodiments, the anti-CDON antibodies, or antigen-binding fragments thereof, can be modified for increased expression in insect cells. In some embodiments, the anti-CDON antibodies, or antigen-binding fragments thereof, can be modified for increased expression in mammalian cells, such as CHO cells.

In some embodiments, the anti-CDON antibodies, or antigen-binding fragments thereof, can be modified to increase stability of the antibodies during production. In some embodiments, the anti-CDON antibodies, or antigen-binding fragments thereof, can be modified to replace one or more amino acids such as asparagine or glutamine that are susceptible to nonenzymatic deamidation with amino acids that do not undergo deamidation (see, Huang et al., Anal. Chem., 2005, 77, 1432-1439). In some embodiments, the anti-CDON antibodies, or antigen-binding fragments thereof, can be modified to replace one or more amino acids that are susceptible to oxidation, such as methionine, cysteine or tryptophan, with an amino acid that does not readily undergo oxidation. In some embodiments, the anti-CDON antibodies, or antigen-binding fragments thereof, can be modified to replace one or more amino acids that are susceptible to cyclization, such as asparagine or glutamic acid, with an amino acid that does not readily undergo cyclization.

In some embodiments, the anti-CDON antibodies, or antigen-binding fragments thereof, have a high binding affinity for CDON. In some embodiments, the anti-CDON antibodies, or antigen-binding fragments thereof, have specific association rate constants (kₒₙ or k_{A} values), dissociation rate constants (k_{off} or k_{D} values), affinity constants (K_{A} values), dissociation constants (K_{D} values) and/or IC₅₀ values. Affinity of anti-CDON antibodies for human CDON can be determined using ELISA, isothermal titration calorimetry (ITC), surface plasmon resonance, or fluorescent polarization assay.

In some embodiments, the anti-CDON antibodies, or antigen-binding fragments thereof, bind to CDON with a K_{A} (kₒₙ/k_{off}) of at least about 10¹⁰ M⁻¹, at least about 4×10¹¹ M⁻¹, at least about 10¹¹ M⁻¹, at least about 4×10¹² M⁻¹, at least about 10¹² M⁻¹, at least about 4×10¹³ M⁻¹, at least about 10¹³ M⁻¹, at least about 4×10¹⁴, at least about 10¹⁴ M⁻¹, at least about 4×10¹⁵ M⁻¹, or with a K_{A} of any range between any pair of the foregoing values (e.g., about 4×10¹¹ M⁻¹ to about 4×10¹³ M⁻¹ or about 4×10¹² M⁻¹ to about 4×10¹⁵ M⁻¹).

In some embodiments, the anti-CDON antibodies, or antigen-binding fragments thereof, bind to CDON with a K_{D} (k_{off}/kₒₙ) of about 10⁻¹⁰ or less, about 4×10⁻¹¹ M or less, about 10⁻¹¹ M or less, about 4×10⁻¹² M or less, about 10⁻¹² M or less, about 4×10¹³ M or less, about 10⁻¹³ M or less, about 4×10¹⁴ M or less, about 10⁻¹⁴ M or less, about 4×10⁻¹⁵ M or less, about 10⁻¹⁵ M or less, or with a K_{D} of any range between any pair of the foregoing values (e.g., about 4×10⁻¹¹ M to about 4×10⁻¹³ M or about 4×10⁻¹² M to about 4×10⁻¹⁵ M).

In some embodiments, the K_{D} (k_{off}/kₒₙ) value is determined by ELISA, isothermal titration calorimetry (ITC), fluorescent polarization assay, or any other biosensor such as BIAcore.

In some embodiments, the anti-CDON antibodies, or antigen-binding fragments thereof, bind to CDON and inhibits the binding of CDON to its ligand at an IC₅₀ less than about 0.02 nM, less than about 0.01 nM, less than about 0.005 nM, less than about 0.002 nM, less than about 0.001 nM, less than about 5×10⁻⁴ nM, less than about 2×10⁻⁴ nM, less than about 1×10⁻⁴ nM, less than about 5×10⁻⁵ nM, less than about 2×10⁻⁵ nM, less than about 1×10⁻⁴ nM, less than about 5×10⁻⁶ nM, less than about 2×10⁻⁶ nM, less than about 1×10⁻⁶ nM, less than about 5×10⁻⁷ nM, less than about 2×10⁻⁷ nM, less than about 1×10⁻⁷ nM, or with an IC₅₀ of any range between any pair of the foregoing values (e.g., about 0.02 nM to about 2×10⁻⁵ nM, or about 5×10⁻⁵ nM to about 1×10⁻⁷ nM). IC₅₀ can be measured according to, for example, ELISA.

The present disclosure also provides compositions comprising any one or more of the anti-CDON antibodies, or antigen-binding fragments thereof, described herein. In some embodiments, the compositions comprise at least two, at least three, or at least four of the anti-CDON antibodies, or antigen-binding fragments thereof, described herein. In some embodiments, the compositions comprise the anti-CDON antibodies, or antigen-binding fragments thereof, and one or more pharmaceutically acceptable carriers and/or excipients. In some embodiments, the carrier(s) and/or excipient(s) is pharmaceutically acceptable for use in humans. Suitable formulations include aqueous and non-aqueous sterile injection solutions which can contain anti-oxidants, buffers, bacteriostats, bactericidal antibiotics, and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of a sterile liquid carrier, for example water for injections, immediately prior to use. Some exemplary ingredients are sodium dodecyl sulfate (SDS) in the range of about 0.1 to about 10 mg/ml, or about 2.0 mg/ml; and/or mannitol or another sugar in the range of about 10 to about 100 mg/ml, or about 30 mg/ml; and/or phosphate-buffered saline (PBS). Any other agents conventional in the art having regard to the type of formulation can be used.

The present disclosure also provides compositions comprising the anti-CDON antibodies, or antigen-binding fragments thereof, conjugated to an active agent, wherein the active agent comprises a therapeutic moiety, a diagnostic moiety, and/or a biologically active moiety. As used herein, the phrase “active agent” refers to a component of the presently disclosed compositions that provides a therapeutic benefit to a subject, permits visualization of cells or tissues in which the compositions of the presently disclosed subject matter accumulate, detection of epitopes to which the presently disclosed antibodies and fragments. In some embodiments, an active agent is selected from the group consisting of a antineoplastic agents, drugs, toxins (including cytotoxins), biologically active proteins, for example, enzymes, anti-angiogenic agents, anti-tumor agents, chemotherapeutic agents, immunomodulators, cytokines, reporter groups, sensitizing molecules other antibody or antibody fragments, synthetic or naturally occurring polymers, nucleic acids (e.g., DNA and RNA), radionuclides, particularly radioiodide, radioisotopes, chelated metals, nanoparticles, reporter groups such as fluorescent compounds, compounds which can be detected by NMR or ESR spectroscopy, or other detectable or imaging agents and combinations thereof. It is understood that these categories are not intended to be mutually exclusive, as some radioactive molecules, for example, are also chemotherapeutic agents, some immunomodulators are cytokines, etc.

The active agent can be a protein or polypeptide, optionally further conjugated to a signaling molecule (such as α-interferon, β-interferon, nerve growth factor, platelet derived growth factor or tissue plasminogen activator), a thrombotic agent or an anti-angiogenic agent or a biological response modifier such as a cytokine or growth factor (e.g., interleukin-1 (IL-1), interleukin-2 (IL-2), interleukin-6 (IL-6), granulocyte macrophage colony stimulating factor (GM-CSF), granulocyte colony stimulating factor (G-CSF), or nerve growth factor (NGF)).

Active agents may be directly or indirectly attached to the polypeptide or antibody. For indirect attachment of a detectable or cytotoxic molecule, the detectable or cytotoxic molecule can be conjugated with a member of a complementary/anti complementary pair, where the other member is bound to the polypeptide or antibody portion. For these purposes, biotin/streptavidin is an exemplary complementary/anti complementary pair.

Suitable detectable agents include, without limitation, radionuclides, enzymes, substrates, cofactors, inhibitors, fluorescent markers, chemiluminescent markers, magnetic particles, and the like.

Suitable cytotoxic agents include, without limitation, Russell's Viper Venom, activated Factor IX, activated Factor X, thrombin, phospholipase C, cobra venom factor, ricin, ricin A chain, Pseudomonas exotoxin, diphtheria toxin, bovine pancreatic ribonuclease, pokeweed antiviral protein (PAP), abrin, abrin A chain, gelonin, saporin, modeccin, viscumin, volkensin, ethidium bromide or PE40, PE38, RNAse, peptide nucleic acids (PNAs), ribosome inactivating protein (RIP) type-1 or type-2, bryodin, momordin, bouganin taxol, cytochalasin B, gramicidin D, ethidium bromide, emetine, mitomycin, etoposide, tenoposide, vincristine, vinblastine, colchicin, doxorubicin, daunorabicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof and combinations thereof, as well as therapeutic radionuclides (either directly attached to the polypeptide or antibody, or indirectly attached through means of a chelating moiety, for instance).

In some embodiments, an active agent comprises a chemotherapeutic. Various chemotherapeutics are known to one of ordinary skill in the art, and include, but are not limited to, alkylating agents such as nitrogen mustards (e.g., Chlorambucil, Cyclophosphamide, Isofamide, Mechlorethamine, Melphalan, Uracil mustard), aziridines (e.g., Thiotepa), methanesulfonate esters (e.g., Busulfan), nitroso ureas (e.g., Carmustine, Lomustine, Streptozocin), platinum complexes (e.g., Cisplatin, Carboplatin), and bioreductive alkylators (e.g., Mitomycin C, Procarbazine); DNA strand breaking agents (e.g., Bleomycin); DNA topoisomerase I inhibitors (e.g., camptothecin and derivatives thereof including, but not limited to 10-hydroxycamptothecin), DNA topoisomerase II inhibitors (e.g., Amsacrine, Dactinomycin, Daunorubicin, Doxorubicin, Idarubicin, Mitoxantrone, Etoposide, Teniposide, Podophyllotoxin); DNA minor groove binders (e.g., Plicamycin); anti-metabolites such as folate antagonists (e.g., Methotrexate and trimetrexate), pyrimidine antagonists (e.g., Fluorouracil, Fluorodeoxyuridine, CB3717, Azacytidine, Cytarabine, Floxuridine), purine antagonists (e.g., Mercaptopurine, 6-Thioguanine, Fludarabine, Pentostatin), sugar modified analogs (e.g., Cyctrabine, Fludarabine), and ribonucleotide reductase inhibitors (e.g., Hydroxyurea); tubulin interactive agents (e.g., Vincristine, Vinblastine, Paclitaxel); adrenal corticosteroids (e.g., Prednisone, Dexamethasone, Methylprednisolone, Prednisolone); hormonal blocking agents such as estrogens and related compounds (e g., Ethinyl Estradiol, Diethylstilbesterol, Chlorotrianisene, Idenestrol), progestins (e.g., Hydroxyprogesterone caproate, Medroxyprogesterone, Megestrol), androgens (e.g., Testosterone, Testosterone propionate; Fluoxymesterone, Methyltestosterone), leutinizing hormone releasing hormone agents and/or gonadotropin-releasing hormone antagonists (e.g., Leuprolide acetate; Goserelin acetate), anti-estrogenic agents (e.g., Tamoxifen), anti-androgen agents (e.g., Flutamide), and anti-adrenal agents (e.g., Mitotane, Aminoglutethimide). Other chemotherapeutics include, but are not limited to Taxol, retinoic acid and derivatives thereof (e.g., 13-cis-retinoic acid, all-trans-retinoic acid, and 9-cis-retinoic acid), sulfathiazole, mitomycin C, mycophenolic acid, sulfadiethoxane, and gemcitabine (4-amino-1-(2-deoxy-2,2-difluoro-.beta.-D-erythro-pentofuranosyl)pyrimidi-n-2(1H)-on-2′,2′-difluoro-2′-deoxycytidine), central nervous system depressants, e.g., general anesthetics (barbiturates, benzodiazepines, steroids, cyclohexanone derivatives, and miscellaneous agents), sedative-hypnotics (benzodiazepines, barbiturates, piperidinediones and triones, quinazoline derivatives, carbamates, aldehydes and derivatives, amides, acyclic ureides, benzazepines and related drugs, phenothiazines, etc.), central voluntary muscle tone modifying drugs (anticonvulsants, such as hydantoins, barbiturates, oxazolidinediones, succinimides, acylureides, glutarimides, benzodiazepines, secondary and tertiary alcohols, dibenzazepine derivatives, valproic acid and derivatives, GABA analogs, etc.), antiproliferative agents, e.g. actinomycin D as well as derivatives and analogs thereof or COSMEGEN, angiopeptin, angiotensin converting enzyme inhibitors such as captopril (e.g., CAPOTEN and CAPOZIDE), cilazapril or lisinopril (e.g., PRINIVIL and PRINZIDE); calcium channel blockers (such as nifedipine), colchicine, fibroblast growth factor (FGF) antagonists, fish oil (ω3-fatty acid), histamine antagonists, lovastatin (an inhibitor of HMG-CoA reductase, a cholesterol lowering drug, MEVACOR), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, and triazolopyrimidine (a PDGF antagonist).

In some embodiments, an active agent comprises an anti-angiogenic agent (e.g., angiostatin or endostatin). Various anti-angiogenic agents are known to one of ordinary skill in the art, and include, but are not limited to inhibitors and/or antagonists of vascular endothelial growth factor (VEGF) family and its receptors (e.g., Bevacizumab and other anti-vascular endothelial growth factor (VEGF) antibodies) and neuropilin-1 antagonists.

Active agents also include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C5 and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, anthramycin (AMC), calicheamicins or duocarmycins), and anti-mitotic agents (e.g., vincristine and vinblastine).

Other active agents can include radionuclides such as, but not limited to ¹³N, ¹⁸F, ³²P ⁶⁴Cu, ⁶⁶Ga, ⁶⁷Ga, ⁶⁸Ga, ⁶⁷Cu, ⁷⁷Br, ^{80m}Br, ⁸²Rb, ⁸⁶Y, ⁹⁰Y, ⁹⁵Ru, ⁹⁷Ru, ^{99m}Tc, ¹⁰³Ru, ¹⁰⁵Ru, ¹¹¹In, ^{113m}In, ¹¹³Sn, ^{121m}Te, ^{122m}Te, ^{125m}Te, ¹²³I, ¹²⁴I, ¹²⁵I, ¹²⁶I, ¹³¹I, ¹³³I, ¹⁶⁵Tm, ¹⁶⁷Tm, ¹⁶⁸Tm, ¹⁷⁷Lu, ¹⁸⁶Re, ¹⁸⁸Re, ^{195m}Hg, ²¹¹At, ²¹²Bi, ²¹³Bi, and ²²⁵Ac.

Active agents also include, but are not limited to, therapeutic agents, such as psychopharmacological agents, such as: 1) analgesics (morphine and derivatives, oripavine derivatives, morphinan derivatives, phenylpiperidines, 2,6-methane-3-benzazocaine derivatives, diphenylpropylamines and isosteres, salicylates, p-aminophenol derivatives, 5-pyrazolone derivatives, arylacetic acid derivatives, fenamates and isosteres, etc.) and antiemetics (anticholinergics, antihistamines, antidopaminergics, etc.); 2) central nervous system stimulants, e.g., analeptics (respiratory stimulants, convulsant stimulants, psychomotor stimulants), narcotic antagonists (morphine derivatives, oripavine derivatives, 2,6-methane-3-benzoxacine derivatives, morphinan derivatives) nootropics; 3) psychopharmacologicals, e.g., anxiolytic sedatives (benzodiazepines, propanediol carbamates) antipsychotics (phenothiazine derivatives, thioxanthine derivatives, other tricyclic compounds, butyrophenone derivatives and isosteres, diphenylbutylamine derivatives, substituted benzamides, arylpiperazine derivatives, indole derivatives, etc.), antidepressants (tricyclic compounds, MAO inhibitors, etc.); 4) respiratory tract drugs, e.g., central antitussives (opium alkaloids and their derivatives); pharmacodynamic agents, such as: a) peripheral nervous system drugs, e.g., local anesthetics (ester derivatives, amide derivatives); b) drugs acting at synaptic or neuroeffector junctional sites, e.g., cholinergic agents, cholinergic blocking agents, neuromuscular blocking agents, adrenergic agents, antiadrenergic agents; c) smooth muscle active drugs, e.g., spasmolytics (anticholinergics, musculotropic spasmolytics), vasodilators, smooth muscle stimulants; and d) histamines and antihistamines, e.g., histamine and derivative thereof (betazole), antihistamines (H₁-antagonists, H₂-antagonists), histamine metabolism drugs; 5) cardiovascular drugs, e.g., cardiotonics (plant extracts, butenolides, pentadienolids, alkaloids from erythrophleum species, ionophores, adrenoceptor stimulants, etc), antiarrhythmic drugs, antihypertensive agents, antilipidemic agents (clofibric acid derivatives, nicotinic acid derivatives, hormones and analogs, antibiotics, salicylic acid and derivatives), antivaricose drugs, hemostyptics; 6) blood and hemopoietic system drugs, e.g., antianemia drugs, blood coagulation drugs (hemostatics, anticoagulants, antithrombotics, thrombolytics, blood proteins and their fractions); 7) gastrointestinal tract drugs, e.g., digestants (stomachics, choleretics), antiulcer drugs, antidiarrheal agents; and 8) locally acting drugs; chemotherapeutic agents, such as: a) anti-infective agents, e.g., ectoparasiticides (chlorinated hydrocarbons, pyrethins, sulfurated compounds), anthelmintics, antiprotozoal agents, antimalarial agents, antiamebic agents, antileiscmanial drugs, antitrichomonal agents, antitrypanosomal agents, sulfonamides, antimycobacterial drugs, antiviral chemotherapeutics, etc.; and b) cytostatics, i.e., antineoplastic agents or cytotoxic drugs, such as alkylating agents, e.g., Mechlorethamine hydrochloride (Nitrogen Mustard, Mustargen, HN2), Cyclophosphamide (Cytovan, Endoxana), Ifosfamide (IFEX), Chlorambucil (Leukeran), Melphalan (Phenylalanine Mustard, L-sarcolysin, Alkeran, L-PAM), Busulfan (Myleran), Thiotepa (Triethylenethiophosphoramide), Carmustine (BiCNU, BCNU), Lomustine (CeeNU, CCNU), Streptozocin (Zanosar) and the like; plant alkaloids, e.g., Vincristine (Oncovin), Vinblastine (Velban, Velbe), Paclitaxel (Taxol), and the like; anti metabolites, e.g., Methotrexate (MTX), Mercaptopurine (Purinethol, 6-MP), Thioguanine (6-TG), Fluorouracil (5-FU), Cytarabine (Cytosar-U, Ara-C), Azacitidine (Mylosar, 5-AZA) and the like; antibiotics, e.g., Dactinomycin (Actinomycin D, Cosmegen), Doxorubicin (Adriamycin), Daunorubicin (duanomycin, Cerubidine), Idarubicin (Idamycin), Bleomycin (Blenoxane), Picamycin (Mithramycin, Mithracin), Mitomycin (Mutamycin) and the like, and other anticellular proliferative agents, e.g., Hydroxyurea (Hydrea), Procarbazine (Mutalane), Dacarbazine (DTIC-Dome), Cisplatin (Platinol) Carboplatin (Paraplatin), Asparaginase (Elspar) Etoposide (VePesid, VP-16-213), Amsarcrine (AMSA, m-AMSA), Mitotane (Lysodren), Mitoxantrone (Novatrone), taxoids, alkylphosphocholines, and the like.

Also included are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX tamoxifen), EVISTA raloxifene, droloxifene, 4-hydroxytamoxifen, trioxifene, keoxifene, LY117018, onapristone, and FARESTON toremifene; anti-progesterones; estrogen receptor down-regulators (ERDs); agents that function to suppress or shut down the ovaries, for example, leutinizing hormone-releasing hormone (LHRH) agonists such as LUPRON and ELIGARD leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, MEGASE megestrol acetate, AROMASIN. exemestane, formestanic, fadrozole, RIVISOR vorozole, FEMARA letrozole, and ARIMIDEX anastrozole. In addition, chemotherapeutic agents include bisphosphonates such as clodronate (for example, BONEFOS or OSTAC), DIDROCAL etidronate, NE-58095, ZOMETA zoledronic acid/zoledronate, FOSAMAX alendronate, AREDIA pamidronate, SKELID tiludronate, or ACTONEL risedronate; as well as troxacitabinc (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE vaccine and gene therapy vaccines, for example, ALLOVECTIN vaccine, LEUVECTIN vaccine, and VAXID vaccine; LURTOTECAN topoisomerase 1 inhibitor; ABARELIX rmRH; lapatinib ditosylate (an ErbB-2 and EGFR dual tyrosine kinase small-molecule inhibitor also known as GW572016); and pharmaceutically acceptable salts, acids or derivatives of any of the above.

Techniques for conjugating such effector moieties to antibodies are well known in the art (see, e.g., Hellstrom et al., Controlled Drug Delivery, 2nd Ed., at pages 623-53 (Robinson et al., eds., 1987)); Thorpe et al., Immunol. Rev., 1982, 62, 119-58; and Dubowchik et al., Pharmacology and Therapeutics, 1999, 83, 67-123).

Active agents also include immunomodulatory agents. Such agents may increase or decrease production of one or more cytokines, up- or down-regulate self-antigen presentation, mask MHC antigens, or promote the proliferation, differentiation, migration, or activation state of one or more types of immune cells. Immunomodulatory agents include but are not limited to: non-steroidal anti-inflammatory drugs (NSAIDs) such as aspirin, ibuprofen, celecoxib, diclofenac, etodolac, fenoprofen, indomethacin, ketoralac, oxaprozin, nabumentone, sulindac, tolmentin, rofecoxib, naproxen, ketoprofen, and nabumetone; steroids (e.g. glucocorticoids, dexamethasone, cortisone, hydroxy cortisone, methylprednisolone, prednisone, prednisolone, trimcinolone, azulfidineicosanoids such as prostaglandins, thromboxanes, and leukotrienes; as well as topical steroids such as anthralin, calcipotriene, clobetasol, and tazarotene); cytokines such as TGFb, IFNa, IFNb, IFNg, IL-2, IL-4, IL-10; cytokine, chemokine, or receptor antagonists including antibodies, soluble receptors, and receptor-Fc fusions against BAFF, B7, CCR2, CCR5, CD2, CD3, CD4, CD6, CD7, CD8, CD11, CD14, CD15, CD17, CD18, CD20, CD23, CD28, CD40, CD40L, CD44, CD45, CD52, CD64, CD80, CD86, CD147, CD152, complement factors (C5, D) CTLA4, eotaxin, Fas, ICAM, ICOS, IFN-a IFN-β, IFN-γ, IFNAR, IgE, IL-1, IL-2, IL-2R, IL-4, IL-5R, IL-6, IL-8, IL-9 IL-12, IL-13, IL-13R1, IL-15, IL-18R, IL-23, integrins, LFA-1, LFA-3, MHC, selectins, TGF-β, TNF-a, TNF-β, TNF-R1, T-cell receptor, including Enbrel®. (etanercept), Humira®. (adalimumab), and Remicade®. (infliximab); heterologous anti-lymphocyte globulin; other immunomodulatory molecules such as 2-amino-6-aryl-5 substituted pyrimidines, anti-idiotypic antibodies for MHC binding peptides and MHC fragments, azathioprine, brequinar, bromocryptine, cyclophosphamide, cyclosporine A, D-penicillamine, deoxyspergualin, FK506, glutaraldehyde, gold, hydroxychloroquine, leflunomide, malononitriloamides (e.g. leflunomide), methotrexate, minocycline, mizoribine, my cophenolate mofetil, rapamycin, and sulfasasazine.

In some embodiments, the anti-CDON antibodies, or antigen-binding fragments thereof, is fused via a covalent bond (e.g., a peptide bond), through the antibody's N-terminus or C-terminus or internally, to an amino acid sequence of another protein (or portion thereof; for example, at least a 10, 20 or 50 amino acid portion of the protein). The antibody, or fragment thereof, can linked to the other protein at the N-terminus of the constant domain of the antibody. Recombinant DNA procedures can be used to create such fusions, for example, as described in WO 86/01533 and EP0392745. In another example, the effector molecule can increase half-life in vivo, and/or enhance the delivery of an antibody across an epithelial barrier to the immune system. Examples of suitable effector molecules of this type include polymers, albumin, albumin binding proteins or albumin binding compounds such as those described in WO 2005/117984.

In some embodiments, the anti-CDON antibodies, or antigen-binding fragments thereof, are conjugated to a small molecule toxin. In some embodiments, the anti-CDON antibodies, or antigen-binding fragments thereof, are conjugated to a dolostatin or a dolastatin peptidic analogs or derivatives, e.g., an auristatin (U.S. Pat. Nos. 5,635,483 and 5,780,588). The dolastatin or auristatin drug moiety may be attached to the antibody through its N-terminus, C-terminus or internally (see, WO 02/088172). Exemplary auristatin embodiments include the N-terminus linked monomethylauristatin drug moieties DE and DF, as disclosed in U.S. Pat. No. 7,498,298 (disclosing, e.g., linkers and methods of preparing monomethylvaline compounds such as MMAE and MMAF conjugated to linkers).

In some embodiments, small molecule toxins include, but are not limited to, calicheamicin, maytansine (U.S. Pat. No. 5,208,020), trichothene, and CC1065. In some embodiments, the antibody is conjugated to one or more maytansine molecules (e.g., about 1 to about 10 maytansine molecules per antibody molecule). Maytansine may, for example, be converted to May-SS-Me which may be reduced to May-SH3 and reacted with an antibody (see, Chari et al., Cancer Res., 1992, 52, 127-131) to generate a maytansinoid-antibody or maytansinoid-Fc fusion conjugate. Structural analogues of calicheamicin that can also be used include, but are not limited to, γ₁¹, γ₃¹, γ₃¹-N-acetyl-γ₁¹, PSAG, and 0₁¹ (Hinman et al., Cancer Res., 1993, 53, 3336-3342; Lode et at, Cancer Res., 1998, 58, 2925-2928; U.S. Pat. Nos. 5,714,586; 5,712,374; 5,264,586; and 5,773,001).

The anti-CDON antibodies, or antigen-binding fragments thereof, disclosed herein can also be conjugated to liposomes for targeted delivery (see, e.g., Park et al., Adv. Pharmacol., 1997, 40, 399-435; and Marty & Schwendener, Methods in Molec. Med., 2004, 109, 389-401).

In some embodiments, the anti-CDON antibodies, or antigen-binding fragments thereof, can be attached to poly(ethyleneglycol) (PEG) moieties. In some embodiments, the anti-CDON antibodies, or antigen-binding fragments thereof, and the PEG moieties can be attached through any available amino acid side-chain or terminal amino acid functional group located in the antibody fragment, for example, any free amino, imino, thiol, hydroxyl or carboxyl group. Such amino acids can occur naturally in the antibody fragment or can be engineered into the fragment using recombinant DNA methods. See for example, U.S. Pat. No. 5,219,996. Multiple sites can be used to attach two or more PEG molecules. PEG moieties can be covalently linked through a thiol group of at least one cysteine residue located in the antibody fragment. Where a thiol group is used as the point of attachment, appropriately activated effector moieties, for example, thiol selective derivatives such as maleimides and cysteine derivatives, can be used.

In some embodiments, the anti-CDON antibodies, or antigen-binding fragments thereof, conjugate are modified Fab′ fragments which are PEGylated, i.e., has PEG (poly(ethyleneglycol)) covalently attached thereto. PEG can be attached to a cysteine in the hinge region. In some embodiments, a PEG-modified Fab′ fragment has a maleimide group covalently linked to a single thiol group in a modified hinge region. A lysine residue can be covalently linked to the maleimide group and to each of the amine groups on the lysine residue can be attached a methoxypoly(ethyleneglycol) polymer having a molecular weight of approximately 20,000 Da. The total molecular weight of the PEG attached to the Fab′ fragment can therefore be approximately 40,000 Da.

As used herein, the term “label’ refers to a detectable compound or composition which can be conjugated directly or indirectly to the anti-CDON antibodies, or antigen-binding fragments thereof. The label can itself be detectable (e.g., radioisotope labels or fluorescent labels) or, in the case of an enzymatic label, can catalyze chemical alteration of a substrate compound or composition which is detectable. Useful fluorescent moieties include, but are not limited to, fluorescein, fluorescein isothiocyanate, rhodamine, 5-dimethylamine-1-napthalenesulfonyl chloride, phycoerythrin and the like. Useful enzymatic labels include, but are not limited to, alkaline phosphatase, horseradish peroxidase, glucose oxidase and the like.

Additional anti-CDON antibody conjugates that are useful for, inter alia, diagnostic purposes, are described below.

The present disclosure also provides methods of making an antibody specific for CDON protein, comprising immunizing an animal with: a) a polypeptide consisting of amino acids at positions corresponding to positions 1 to 200 according to SEQ ID NO:1; or b) a polypeptide consisting of amino acids at positions corresponding to positions 1000 to 1287 according to SEQ ID NO: 1.

The present disclosure also provides methods of making an antibody specific for CDON protein, comprising immunizing an animal with: a) a polypeptide consisting of amino acids at positions corresponding to positions 100 to 200 according to SEQ ID NO: 1; or b) a polypeptide consisting of amino acids at positions corresponding to positions 1200 to 1287 according to SEQ ID NO: 1.

The present disclosure also provides methods of making an antibody specific for CDON protein, comprising immunizing an animal with: a) a polypeptide consisting of amino acids at positions corresponding to positions 140 to 170 according to SEQ ID NO: 1; or b) a polypeptide consisting of amino acids at positions corresponding to positions 1250 to 1287 according to SEQ ID NO: 1.

The present disclosure also provides methods of making an antibody specific for CDON protein, comprising immunizing an animal with: a) a polypeptide consisting of the amino acid sequence RVPESNPKAEVRYKIRGK (SEQ ID NO:2); b) a polypeptide consisting of the amino acid sequence GIPLDSPTEVLQQPRET (SEQ ID NO: 3); c) a polypeptide consisting of the amino acid sequence VLGDFGSSTKHVITAEE (SEQ ID NO:4); and/or d) a polypeptide consisting of the amino acid sequence KIRGKWLEHSTENY (SEQ ID NO:5).

In some embodiments, polyclonal antibodies are raised in animals by multiple subcutaneous (sc) or intraperitoneal (ip) injections of immunogenic form of the peptide which elicits an antibody response in the mammal (e g., RVPESNPKAEVRYKIRGK (SEQ ID NO:2); GIPLDSPTEVLQQPRET (SEQ ID NO:3); VLGDFGSSTKHVITAEE (SEQ ID NO:4); or KIRGKWLEHSTENY (SEQ ID NO:5)). Techniques for conferring immunogenicity on a peptide include conjugation to carriers. For example, it may be useful to conjugate the relevant antigen (especially when synthetic peptides are used) to a protein that is immunogenic in the species to be immunized. For example, the antigen can be conjugated to keyhole limpet hemocyanin (KLH; e g., KLH-EG and KLH-M), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups. The progress of immunization can be monitored by detection of antibody titers in plasma or serum. Standard ELISA or other immunoassay procedures are optionally used with the immunogen as antigen to assess the levels of antibodies.

Immunization of animals can be carried out by any one of several techniques (see, e.g., Harlow and Lane, Antibodies: A Laboratory Manual, New York: Cold Spring Harbor Press, 1990). Methods for immunizing non-human animals such as mice, rats, sheep, goats, pigs, cattle and horses can be carried out by any one of several techniques (see, e.g., Harlow and Lane and U.S. Pat. No. 5,994,619). In some embodiments, the CDON antigen is administered with an adjuvant to stimulate the immune response. Such adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. In some embodiments, if a polypeptide is being administered, the immunization schedule will involve two or more administrations of the polypeptide, spread out over several weeks.

After immunization of an animal with a CDON antigen, antibodies and/or antibody-producing cells may be obtained from the animal by any one of several techniques. An anti-CDON antibody-containing serum is obtained from the animal by bleeding or sacrificing the animal. The serum may be used as it is obtained from the animal, an immunoglobulin fraction may be obtained from the serum, or the anti-CDON antibodies may be purified from the serum using standard methods such as plasmaphoresis or adsorption chromatography with IgG-specific adsorbents such as immobilized Protein A. Serum or immunoglobulins obtained in this manner are polyclonal, which are disadvantageous because the amount of antibodies that can be obtained is limited and the polyclonal antibody has a heterogeneous array of properties.

Monoclonal antibodies can be prepared using the hybridoma method first described by Kohler et al., Nature, 1975, 256, 495, or may be made by recombinant DNA methods (see, U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (see, Goding, Monoclonal Antibodies: Principles and Practice, pp. 59-103 (Academic Press, 1986)).

The prepared hybridoma cells are seeded and grown in a suitable culture medium which medium that, for example, contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Suitable fusion partner myeloma cells are those that fuse efficiently, support stable high-level production of antibody by the selected antibody-producing cells, and are sensitive to a selective medium that selects against the unfused parental cells. Suitable myeloma cell lines are murine myeloma lines, such as those derived from MOPC-21 and MPC-11 mouse tumors available from the Salk Institute Cell Distribution Center, San Diego, Calif USA, and SP-2 and derivatives e.g., X63-Ag8-653 cells available from the American Type Culture Collection, Manassas, Va., USA. Human myeloma and mouse-human heteromyeloma cell lines also have been described for the production of human monoclonal antibodies (Kozbor, J. Immunol., 1984, 133, 3001; and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. In some embodiments, the binding specificity of monoclonal antibodies produced by hybridoma cells is determined by immunoprecipitation or by an in vitro binding assay, such as radioimmunoassay (RIA) or ELISA. The binding affinity of a monoclonal antibody can, for example, be determined by the Scatchard analysis described in Munson et al., Anal. Biochem., 1980, 107, 220.

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal e.g., by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies can be isolated and sequenced using conventional procedures (e.g., by using oligonucleotide probes that are capable of binding specifically to genes encoding the heavy and light chains of murine antibodies). The hybridoma cells serve as a suitable source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells (see, Skerra et al., Curr. Opinion in Immunol., 1993, 5, 25 6-262 and Pluckthun, Immunol. Revs., 1992, 130, 151-188).

In some embodiments, monoclonal antibodies or antibody fragments can be isolated from antibody phage libraries generated using the techniques described in McCafferty et al., Nature, 1990, 348, 552-554. Clackson et al., Nature, 1991, 352, 624-628 and Marks et al., J. Mol. Biol., 1991, 222, 581-597 describe the isolation of murine and human antibodies, respectively, using phage libraries. Subsequent publications describe the production of high affinity (nM range) human antibodies by chain shuffling (Marks et al., Bio/Technology, 1992, 10, 779-783), as well as combinatorial infection and in vivo recombination as a strategy for constructing very large phage libraries (Waterhouse et al., Nuc. Acids. Res., 1993, 21, 2265-2266). Thus, these techniques are viable alternatives to traditional monoclonal antibody hybridoma techniques for isolation of monoclonal antibodies.

The DNA that encodes the antibody may be modified to produce chimeric or fusion antibody polypeptides, for example, by substituting human heavy chain and light chain constant domain (CH and C_{L}) sequences for the homologous murine sequences (U.S. Pat. No. 4,816,567; and Morrison, et al., Proc. Natl. Acad. Sci. USA., 1984, 81, 6851), or by fusing the immunoglobulin coding sequence with all or part of the coding sequence for a non-immunoglobulin polypeptide (heterologous polypeptide). The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The immunizing peptides may also be produced by recombinant DNA technology. To prepare the CDON-specific epitopes by recombinant DNA techniques, a DNA sequence encoding the CDON-specific epitopes is prepared. Consequently, the present disclosure also includes the use of purified and isolated nucleic acids comprising a nucleotide sequence coding for CDON-specific epitopes to elicit an immune response.

Antibodies specifically reactive with protein epitopes, or derivatives, such as enzyme conjugates or labeled derivatives, are useful to detect protein epitopes in various samples (e.g. biological materials). They are useful as diagnostic or prognostic reagents and are readily used to detect abnormalities in the level of protein expression, or abnormalities in the structure, and/or temporal, tissue, cellular, or subcellular location of protein epitopes. In vitro immunoassays are also useful to assess or monitor the efficacy of particular therapies. The anti-CDON antibodies, or antigen-binding fragments thereof, may also be used in vitro to determine the presence of CDON or the level of expression thereof. Accordingly, anti-CDON antibodies, or antigen-binding fragments thereof, including those antibodies that have been modified, e.g., by biotinylation, horseradish peroxidase, or any other detectable moiety (including those described above), can be advantageously used for diagnostic purposes.

In some embodiments, anti-CDON antibodies, or antigen-binding fragments thereof, can be used, for example, but not limited to, to purify or detect CDON, including both in vitro and in vivo diagnostic methods. For example, anti-CDON antibodies, or antigen-binding fragments thereof, have use in immunoassays for qualitatively and quantitatively measuring levels of CDON in biological samples, or to identify the location, quantity, and/or behavior of CDON in an animal.

Measuring levels of CDON using anti-CDON antibodies, or antigen-binding fragments thereof, may be used to, for example, 1) diagnose (e.g., determine an increased risk of) cancer in patient, 2) determine the prognosis of a patient, including A) stage and grade of a tumor (particularly whether the cancer is metastatic or likely to be metastatic) and/or B) its potential sensitivity to CDON therapy, 3) determine the origin of a tumor, and 4) determine the efficacy of a treatment of a patient.

The present disclosure provides methods for assessing the presence of a tumor in a mammal comprising: a) contacting a test sample containing tissue or cells obtained from the mammal with an anti-CDON antibody, or antigen-binding fragment thereof, that binds to a CDON polypeptide; b) detecting the formation of a complex between anti-CDON antibodies, or antigen-binding fragments thereof, and the CDON polypeptide in the test sample; and c) comparing the formation of a complex in the test sample relative to a control sample, wherein the formation of a greater amount of the complex in the test sample relative to a control sample is indicative of the presence of the tumor in the mammal; wherein anti-CDON antibody, or antigen-binding fragment thereof, specifically binds an isolated peptide selected from: i) the polypeptide consisting of amino acid residues 1 to 200 according to SEQ ID NO: 1; and ii) the polypeptide consisting of amino acid residues 1000 to 1287 according to SEQ ID NO: 1. In some embodiments, the methods further comprise obtaining the test sample comprising tissue or cells from the mammal.

In some embodiments, the methods for assessing the presence of a tumor in a mammal comprises: a) contacting a test sample containing tissue or cells obtained from the mammal with an anti-CDON antibody, or antigen-binding fragment thereof, that binds to a CDON polypeptide; b) detecting the formation of a complex between the anti-CDON antibody, or antigen-binding fragment thereof, and the CDON polypeptide in the test sample; and c) comparing the formation of a complex in the test sample relative to a control sample, wherein the formation of a greater amount of the complex in the test sample relative to a control sample is indicative of the presence of the tumor in the mammal; wherein the anti-CDON antibody, or antigen-binding fragment thereof, specifically binds an isolated peptide selected from: i) RVPESNPKAEVRYKIRGK (SEQ ID NO:2); ii) GIPLDSPTEVLQQPR ET (SEQ ID NO:3); VLGDFGSSTKHVITAEE (SEQ ID NO:4); or KIRGKWLEHSTENY (SEQ ID NO:5). In some embodiments, the methods further comprise obtaining the test sample comprising tissue or cells from the mammal.

The present disclosure also provides methods for detecting the presence or absence of a tumor in a mammal comprising: a) contacting a tissue or cell sample obtained from the mammal with an anti-CDON antibody, or antigen-binding fragment thereof, that specifically binds CDON polypeptide, wherein the anti-CDON antibody, or antigen-binding fragment thereof, specifically binds: i) a CDON polypeptide consisting of amino acids at positions corresponding to positions 1 to 200 according to SEQ ID NO: 1, or ii) a CDON polypeptide consisting of amino acids at positions corresponding to positions 1000 to 1287 according to SEQ ID NO: 1; b) detecting the presence or absence of a complex between the anti-CDON antibody, or antigen-binding fragment thereof, and a CDON polypeptide in the sample; and c) comparing the formation or lack or formation of the complex in the sample with a control sample, wherein the formation of a greater amount of the complex in the sample compared to the control sample indicates the presence of a tumor in the mammal, and wherein the formation of an equal amount or lesser amount of the complex in the sample compared to the control sample indicates the absence of a tumor in the mammal.

The present disclosure also provides methods for detecting the presence or absence of a tumor in a mammal comprising: a) contacting a tissue or cell sample obtained from the mammal with an anti-CDON antibody, or antigen-binding fragment thereof, that specifically binds CDON polypeptide, wherein the anti-CDON antibody, or antigen-binding fragment thereof, specifically binds: i) a CDON polypeptide consisting of amino acids at positions corresponding to positions 100 to 200 according to SEQ ID NO: 1, or ii) a CDON polypeptide consisting of amino acids at positions corresponding to positions 1200 to 1287 according to SEQ ID NO: 1; b) detecting the presence or absence of a complex between the anti-CDON antibody, or antigen-binding fragment thereof, and a CDON polypeptide in the sample; and c) comparing the formation or lack or formation of the complex in the sample with a control sample, wherein the formation of a greater amount of the complex in the sample compared to the control sample indicates the presence of a tumor in the mammal, and wherein the formation of an equal amount or lesser amount of the complex in the sample compared to the control sample indicates the absence of a tumor in the mammal.

The present disclosure also provides methods for detecting the presence or absence of a tumor in a mammal comprising: a) contacting a tissue or cell sample obtained from the mammal with an anti-CDON antibody, or antigen-binding fragment thereof, that specifically binds CDON polypeptide, wherein the anti-CDON antibody, or antigen-binding fragment thereof, specifically binds: i) a CDON polypeptide consisting of amino acids at positions corresponding to positions 140 to 170 according to SEQ ID NO:1, or ii) a CDON polypeptide consisting of amino acids at positions corresponding to positions 1250 to 1287 according to SEQ ID NO: 1; b) detecting the presence or absence of a complex between the anti-CDON antibody, or antigen-binding fragment thereof, and a CDON polypeptide in the sample; and c) comparing the formation or lack or formation of the complex in the sample with a control sample, wherein the formation of a greater amount of the complex in the sample compared to the control sample indicates the presence of a tumor in the mammal, and wherein the formation of an equal amount or lesser amount of the complex in the sample compared to the control sample indicates the absence of a tumor in the mammal.

The present disclosure also provides methods for detecting the presence or absence of a tumor in a mammal comprising: a) contacting a tissue or cell sample obtained from the mammal with an anti-CDON antibody, or antigen-binding fragment thereof, that specifically binds CDON polypeptide, wherein the anti-CDON antibody, or antigen-binding fragment thereof, specifically binds: i) a CDON polypeptide consisting of the amino acid sequence RVPESNPKAEVRYKIRGK (SEQ ID NO:2), ii) a CDON polypeptide consisting of the amino acid sequence GIPLDSPTEVLQQPRET (SEQ ID NO: 3); iii) a CDON polypeptide consisting of the amino acid sequence VLGDFGSSTKHVITAEE (SEQ ID NO:4); or iv) a CDON polypeptide consisting of the amino acid sequence KIRGKWLEHSTENY (SEQ ID NO:5); b) detecting the presence or absence of a complex between the anti-CDON antibody, or antigen-binding fragment thereof, and a CDON polypeptide in the sample; and c) comparing the formation or lack or formation of the complex in the sample with a control sample, wherein the formation of a greater amount of the complex in the sample compared to the control sample indicates the presence of a tumor in the mammal, and wherein the formation of an equal amount or lesser amount of the complex in the sample compared to the control sample indicates the absence of a tumor in the mammal.

The present disclosure also provides methods for determining the presence or absence of CDON polypeptide in a human comprising: a) administering to the human an anti-CDON antibody, or antigen-binding fragment thereof, that specifically binds the CDON polypeptide, wherein the anti-CDON antibody, or antigen-binding fragment thereof, specifically binds: i) a CDON polypeptide consisting of amino acids at positions corresponding to positions 1 to 200 according to SEQ ID NO: 1, or ii) a CDON polypeptide consisting of amino acids at positions corresponding to positions 1000 to 1287 according to SEQ ID NO: 1; wherein the anti-CDON antibody, or antigen-binding fragment thereof, is labeled with a detectable label; and b) externally scanning the human for localization of the labeled anti-CDON antibody, or antigen-binding fragment thereof.

The present disclosure also provides methods for determining the presence or absence of CDON polypeptide in a human comprising: a) administering to the human an anti-CDON antibody, or antigen-binding fragment thereof, that specifically binds the CDON polypeptide, wherein the anti-CDON antibody, or antigen-binding fragment thereof, specifically binds: i) a CDON polypeptide consisting of amino acids at positions corresponding to positions 100 to 200 according to SEQ ID NO: 1, or ii) a CDON polypeptide consisting of amino acids at positions corresponding to positions 1200 to 1287 according to SEQ ID NO: 1; wherein the anti-CDON antibody, or antigen-binding fragment thereof, is labeled with a detectable label; and b) externally scanning the human for localization of the labeled anti-CDON antibody, or antigen-binding fragment thereof.

The present disclosure also provides methods for determining the presence or absence of CDON polypeptide in a human comprising: a) administering to the human an anti-CDON antibody, or antigen-binding fragment thereof, that specifically binds the CDON polypeptide, wherein the anti-CDON antibody, or antigen-binding fragment thereof, specifically binds: i) a CDON polypeptide consisting of amino acids at positions corresponding to positions 140 to 170 according to SEQ ID NO:1, or ii) a CDON polypeptide consisting of amino acids at positions corresponding to positions 1250 to 1287 according to SEQ ID NO: 1; wherein the anti-CDON antibody, or antigen-binding fragment thereof, is labeled with a detectable label; and b) externally scanning the human for localization of the labeled anti-CDON antibody, or antigen-binding fragment thereof.

The present disclosure also provides methods for determining the presence or absence of CDON polypeptide in a human comprising: a) administering to the human an anti-CDON antibody, or antigen-binding fragment thereof, that specifically binds the CDON polypeptide, wherein the anti-CDON antibody, or antigen-binding fragment thereof, specifically binds: i) a CDON polypeptide consisting of the amino acid sequence RVPESNPKAEVRYKIRGK (SEQ ID NO:2), ii) a CDON polypeptide consisting of the amino acid sequence GIPLDSPTEVLQQPRET (SEQ ID NO:3); iii) a CDON polypeptide consisting of the amino acid sequence VLGDFGSSTKHVITAEE (SEQ ID NO:4); or iv) a CDON polypeptide consisting of the amino acid sequence KIRGKWLEHSTENY (SEQ ID NO:5); wherein the anti-CDON antibody, or antigen-binding fragment thereof, is labeled with a detectable label; and b) externally scanning the human for localization of the labeled anti-CDON antibody, or antigen-binding fragment thereof.

The “control” can be a sample from a subject or a group of subjects who are either known as having CDON-expressing cancer or tumor (positive control) or not having CDON-expressing cancer or tumor (negative control). A person skilled in the art will appreciate that the difference in the amount of antibody-antigen complex will vary depending on the control. For example, if the control is known to have CDON-expressing cancer or tumor, then less measurable antibody-antigen complex in the test sample as compared to the control indicates that the subject does not have CDON-expressing cancer or tumor or that they have less of an extent of CDON-expressing cancer or tumor. If the control is known to have CDON-expressing cancer or tumor, then equal or greater measurable antibody-antigen complex in the test sample as compared to the control indicates that the subject has CDON-expressing cancer or tumor. If the control is known not to have CDON-expressing cancer or tumor, then less or equal measurable antibody-antigen complex in the test sample as compared to the control indicates that the subject does not have CDON-expressing cancer or tumor. If the control is known not to have CDON-expressing cancer or tumor, then greater measurable antibody-antigen complex in the test sample as compared to the control indicates that the subject has CDON-expressing cancer or tumor.

In some embodiments, the anti-CDON antibodies, or antigen-binding fragments thereof, can be used, for example, in conjunction with compound screening assays, for the evaluation of the effect of test compounds on expression and/or activity of the CDON gene product. Additionally, such anti-CDON antibodies, or antigen-binding fragments thereof, can be used in conjunction with gene therapy techniques to, for example, evaluate the success of transfection of normal and/or engineered CDON-expression.

In some embodiments, the anti-CDON antibodies, or antigen-binding fragments thereof, can be conjugated to a diagnostic agent. The anti-CDON antibodies, or antigen-binding fragments thereof, can be used diagnostically, for example, to detect expression of a target of interest in specific cells, tissues, or serum; or to monitor the development or progression of an immunologic response as part of a clinical testing procedure to, e.g., determine the efficacy of a particular treatment regimen. Detection can be facilitated by coupling the anti-CDON antibodies, or antigen-binding fragments thereof, to a detectable substance. Examples of detectable substances include various enzymes, prosthetic groups, fluorescent materials (e.g., fluorescein and rhodamine and their derivatives), luminescent materials, bioluminescent materials, optical agents (e.g., derivatives of phorphyrins, anthraquinones, anthrapyrazoles, perylenequinones, xanthenes, cyanines, acridines, phenoxazines and phenothiazines), radioactive materials, positron emitting metals using various positron emission tomographies, and nonradioactive paramagnetic metal ions (e.g., Gd(III), Eu(III), Dy(III), Pr(III), Pa(IV), Mn(II), Cr(III), Co(III), Fe(III), Cu(II), Ni(II), Ti(III), and V(IV)). The detectable substance can be coupled or conjugated either directly to the anti-CDON antibodies, or antigen-binding fragments thereof, or indirectly, through an intermediate (such as, for example, a linker known in the art). Examples of enzymatic labels include luciferases (e.g., fire Drosophila luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, 0-galactosidase, acetylcholinesterase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include 125I, 131I, 111In or 99Tc.

The present disclosure also provides methods for detecting expression of CDON, comprising contacting a biological sample from a patient using one or more anti-CDON antibodies, or antigen-binding fragments thereof, (optionally conjugated to detectable moiety), and detecting whether or not the sample is positive for CDON expression, or whether the sample has altered (e.g., reduced or increased) expression as compared to a control sample. The biological sample may include biopsies of various tissues including, without limitation: skin, muscle, breast, prostate, cervical, ovarian, brain, testicular, and pulmonary. Cellular examples of biological samples include tumor cells, skin cells, muscle cells, blood cells, ovarian cells, brain cells, prostate cells, breast cells, testicular cells, cervical cells, and lung cells. The biological sample may also be a biological fluid.

The present disclosure also provides methods for determining the expression levels of CDON polypeptide in a patient suspected of having a tumor, comprising: a) administering to the patient an anti-CDON antibody, or antigen-binding fragment thereof, that binds to CDON polypeptide, wherein the anti-CDON antibody, or antigen-binding fragment thereof, is labeled with a detectable label, and b) externally scanning the patient for localization of the label; wherein the anti-CDON antibody, or antigen-binding fragment thereof, specifically binds an isolated peptide selected from: i) a CDON polypeptide consisting of amino acid residues 1 to 200 according to SEQ ID NO: 1, and ii) a CDON polypeptide consisting of amino acid residues 1000 to 1287 according to SEQ ID NO: 1.

The present disclosure also provides methods for determining the expression levels of CDON polypeptide in a patient suspected of having a tumor, comprising: a) administering to the patient an anti-CDON antibody, or antigen-binding fragment thereof, wherein the anti-CDON antibody, or antigen-binding fragment thereof, is labeled with a detectable label, and b) externally scanning the patient for localization of the label; wherein the anti-CDON antibody, or antigen-binding fragment thereof, specifically binds an isolated peptide selected from: i) RVPESNPKAEVRYKIRGK (SEQ ID NO:2) or ii) GIPLDSPTEVLQQPRET (SEQ ID NO:3).

The presence of CDON-expressing cells in a biological sample is indicative of the presence of cancer and possibly indicative of metastases, particularly when present in quantities greater than that of normal healthy subjects. The loss of CDON-expressing cells in a patient, particularly one undergoing treatment, over time is indicative of remission (i.e., successful treatment), while the lack of change in CDON-expressing cell levels in a patient undergoing treatment is indicative of resistance to the therapy and indicates that a different therapeutic strategy could be employed. Similarly, the gain of CDON-expressing cells in a patient overtime can be indicative of recurrence. Additionally, the imaging techniques described herein may be employed to monitor the size of the tumor to determine the efficacy of a treatment. In some embodiments, other cancer diagnostic assays can be performed to confirm the results obtained with the methods disclosed herein.

In some embodiments, a biological sample (e.g., a tumor sample) may be obtained from a subject and the presence of CDON-expressing cells determined. The number of CDON-expressing cells may be correlated with tumor grade. In some embodiments, the number of CDON-expressing cells in the biological sample is compared to the number of CDON-expressing cells in a corresponding biological sample from a healthy individual to determine the modulation of CDON-expressing cells in the tumor. Subjects comprising the tumor may be treated with agents to modulate the activity of CDON-expressing cells to normal, healthy levels.

CDON protein levels may be measured using any immunoassays which rely on the binding interaction between an antigenic determinant of the protein epitopes and the antibodies. Examples of such assays are radioimmunoassays, enzyme immunoassays (e.g. ELISA including Sandwich ELISA), immunofluorescence, immunoprecipitation, latex agglutination, hemagglutination, and histochemical tests. The antibodies are useful to detect and quantify the protein in a sample in order to determine its role and to diagnose the disease caused by the protein.

In some embodiments, the anti-CDON antibodies, or antigen-binding fragments thereof, are useful in immunohistochemical analyses, for example, at the cellular and subcellular level, to detect CDON protein, to localize it to particular cells and tissues, and to specific subcellular locations, and to quantitate the level of expression. Cytochemical techniques for localizing antigens include using light and electron microscopy to detect polypeptides such as proteins. Generally, anti-CDON antibodies, or antigen-binding fragments thereof, are optionally labeled with a detectable substance and the recognized polypeptide is localised in tissues and cells based upon the presence of the detectable substance. Examples of detectable substances include, but are not limited to: radioisotopes (e.g., ³H, ¹⁴C, ³⁵S, ³²P, ¹²³I, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or colorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualized by electron microscopy.

The anti-CDON antibodies, or antigen-binding fragments thereof, or sample may be immobilized on a carrier or solid support which is capable of immobilizing cells, antibodies etc. For example, the carrier or support may be nitrocellulose, or glass, polyacrylamides, gabbros, and magnetite. The support material may have any possible configuration including spherical (e.g. bead), cylindrical (e.g., inside surface of a test tube or well, or the external surface of a rod), or flat (e.g., sheet, test strip). Indirect methods may also be employed in which the primary antigen-antibody reaction is amplified by the introduction of a second antibody, having specificity for the antibody reactive against protein epitopes. By way of example, if the antibody having specificity against a polypeptide epitope is a rabbit IgG antibody, the second antibody may be goat anti-rabbit gamma-globulin labeled with a detectable substance as described herein.

Anti-CDON antibodies, or antigen-binding fragments thereof, may also be used for tagging cells that express CDON, for isolating CDON by affinity purification, for diagnostic assays for determining circulating levels of CDON polypeptides, for detecting or quantitating soluble CDON as a marker of underlying pathology or disease, in analytical methods employing FACS, for screening expression libraries, for generating anti-idiotypic antibodies, and as neutralizing antibodies or as antagonists to block CDON activity in vitro and in vivo.

Where a radioactive label is used as a detectable substance, CDON proteins may be localized by autoradiography. The results of autoradiography may be quantitated by determining the density of particles in the autoradiographs by various optical methods, or by counting the grains.

Diseases that can be diagnosed using the present methods include, but are not limited to, cancers including, without limitation, prostate (e.g., adenocarcinoma), bladder, biliary, lung (e.g., small cell or non-small cell), brain, skin, colon, kidney, liver, breast, urogenital, cervical, uterine (e.g., endometrial), ovarian, testicular, cancer of the penis, cancer of the vagina, cancer of the urethra, gall bladder, esophageal or pancreatic. In some embodiments, the cancer is skeletal or smooth muscle, stomach, cancer of the small intestine, cancer of the salivary gland, anal, rectal, thyroid, parathyroid, pituitary, nasopharyngeal, neuronal system cancers (e.g., glioblastoma, malignant glioma, meningioma, medulloblastoma, astrocytoma, neuroectodermal tumors and ependymoma), breast cancer, cancer is inferior ductal carcinoma, inferior lobular carcinoma, intraductal carcinoma, medullary carcinoma and tubular carcinoma, lung cancer, adenocarcinoma, broncho-alveolar adenocarcinoma, squamous cell carcinoma, and small cell carcinoma.

The present disclosure also provides methods of treatment using anti-CDON antibody, or antigen-binding fragments thereof. In some embodiments, the methods involve administering to a human patient having a solid tumor an amount of an anti-CDON antibody, or antigen-binding fragment thereof, that antagonizes CDON, and kills tumor cells at a rate effective to provide a therapeutic benefit.

The anti-CDON antibodies, or antigen-binding fragments thereof, can be used to treat various CDON-expressing neoplasms. In some embodiments, treatment with an anti-CDON antibody, or antigen-binding fragment thereof, results in the inhibition of the proliferation of CDON-expressing cancer cells. Inhibition of cell proliferation and/or self-renewal may lead to improvement in the signs or symptoms of disease. For example, such therapy may result in an improvement in survival (overall survival and/or progression free survival) and/or may result in an objective clinical response (partial or complete). In some embodiments, the anti-CDON antibodies, or antigen-binding fragments thereof, function as antagonists of CDON biological activity, and can additionally be used as a method for the inhibition of abnormal CDON activity. In some embodiments, the anti-CDON antibodies, or antigen-binding fragments thereof, can be used in the therapeutic treatment of cancer where disruption of cell-adhesion is anti-tumorigenic (i.e., clustered cells not only in abdomen, such as gynecologic cancers, but also those that produce circulating tumor cells).

In some embodiments, the anti-CDON antibodies, or antigen-binding fragments thereof, are useful in the treatment of CDON-expressing tumors, including cancers and benign tumors. More particularly, cancers that are amenable to treatment by the anti-CDON antibodies, or antigen-binding fragments thereof, include those that overexpress CDON. In some embodiments, cancers that are amenable to treatment by the antibodies disclosed herein include, but are not limited to, prostate (e.g., adenocarcinoma), bladder, biliary, lung (e.g., small cell or non-small cell), skin, colon, kidney, liver, breast, urogenital, cervical, uterine (e.g., endometrial), ovarian, testicular, cancer of the penis, cancer of the vagina, cancer of the urethra], gall bladder, esophageal or pancreatic. In some embodiments, the cancer is skeletal or smooth muscle, stomach, cancer of the small intestine, cancer of the salivary gland, anal, rectal, thyroid, parathyroid, pituitary, nasopharyngeal, neuronal system cancers (malignant glioma, meningioma, medulloblastoma, neuroectodermal tumors and ependymoma), breast cancer, cancer is inferior ductal carcinoma, inferior lobular carcinoma, intraductal carcinoma, medullary carcinoma and tubular carcinoma, thyroid follicular adenoma, lung cancer, adenocarcinoma, broncho-alveolar adenocarcinoma, vascular endothelium hemangioma, squamous cell carcinoma, and small cell carcinoma. The cancer may be newly diagnosed and naive to treatment, or may be relapsed, refractory, or relapsed and refractory, or a metastatic form of a solid tumor.

In some embodiments, the anti-CDON antibodies, or antigen-binding fragments thereof, are useful in the treatment of a CDON-expressing blood malignancy, including, but not limited to, myelomas (e.g., multiple myeloma), lymphomas (e.g., Hodgkin's lymphoma, non-Hodgkin's lymphoma, Waldenstrom's macroglobulinemia, mantle cell lymphoma), leukemias (e.g., chronic lymphocytic leukemia, acute myeloid leukemia, acute lymphocytic leukemia), and myelodysplastic syndromes. In some embodiments, the methods comprise administering to a human patient having a blood malignancy an amount of an anti-CDON antibody, or antigen-binding fragment thereof, that antagonizes CDON, and kills malignant cells at a rate effective to provide therapeutic benefit.

The present disclosure also provides methods of treating any of the foregoing diseases in a patient in need thereof, comprising: administering to the patient an anti-CDON antibody, or antigen-binding fragment thereof. As demonstrated in the Examples, the addition of the N-terminus antibody described herein to cultured OVCAR3 cells resulted in the induction of apoptosis not observed with a commercially available antibody designed to the N-terminus of CDON (i.e., R&D Catalog #AF4384).

The present disclosure also provides methods for treating a tumor comprising administering to a subject in need of such treatment an effective amount of an anti-CDON antibody, or antigen-binding fragment thereof, wherein the anti-CDON antibody, or antigen-binding fragment thereof, specifically binds an isolated peptide selected from: i) a CDON polypeptide consisting of amino acid residues 1 to 200 according to SEQ ID NO: 1, and ii) a CDON polypeptide consisting of amino acid residues 1000 to 1287 according to SEQ ID NO: 1.

The present disclosure also provides methods for treating a tumor comprising administering to a subject in need of such treatment an effective amount of an anti-CDON antibody, or antigen-binding fragment thereof, that binds to CDON polypeptide, wherein the anti-CDON antibody, or antigen-binding fragment thereof, specifically binds an isolated peptide selected from: i) RVPESNPKAEVRYKIRGK (SEQ ID NO:2); ii) GIPLDSPTEVLQQPRET (SEQ ID NO:3); iii) VLGDFGSSTKHVITAEE (SEQ ID NO:4); or iv) KIRGKWLEHSTENY (SEQ ID NO:5).

The present disclosure also provides methods for treating a human having a tumor comprising administering to the human in need thereof an anti-CDON antibody, or antigen-binding fragment thereof, that specifically binds CDON polypeptide, wherein the anti-CDON antibody, or antigen-binding fragment thereof, specifically binds: i) a CDON polypeptide consisting of amino acids at positions corresponding to positions 1 to 200 according to SEQ ID NO: 1, or ii) a CDON polypeptide consisting of amino acids at positions corresponding to positions 1000 to 1287 according to SEQ ID NO:1.

The present disclosure also provides methods for treating a human having a tumor comprising administering to the human in need thereof an anti-CDON antibody, or antigen-binding fragment thereof, that specifically binds CDON polypeptide, wherein the anti-CDON antibody, or antigen-binding fragment thereof, specifically binds: i) a CDON polypeptide consisting of amino acids at positions corresponding to positions 100 to 200 according to SEQ ID NO: 1, or ii) a CDON polypeptide consisting of amino acids at positions corresponding to positions 1200 to 1287 according to SEQ ID NO:1.

The present disclosure also provides methods for treating a human having a tumor comprising administering to the human in need thereof an anti-CDON antibody, or antigen-binding fragment thereof, that specifically binds CDON polypeptide, wherein the anti-CDON antibody, or antigen-binding fragment thereof, specifically binds: i) a CDON polypeptide consisting of amino acids at positions corresponding to positions 140 to 170 according to SEQ ID NO: 1, or ii) a CDON polypeptide consisting of amino acids at positions corresponding to positions 1250 to 1287 according to SEQ ID NO:1.

The present disclosure also provides methods for treating a human having a tumor comprising administering to the human in need thereof an anti-CDON antibody, or antigen-binding fragment thereof, that specifically binds CDON polypeptide, wherein the anti-CDON antibody, or antigen-binding fragment thereof, specifically binds: i) a CDON polypeptide consisting of the amino acid sequence RVPESNPKAEVRYKIRGK (SEQ ID NO:2); ii) a CDON polypeptide consisting of the amino acid sequence GIPLDSPTEVLQQPRET (SEQ ID NO:3); iii) a CDON polypeptide consisting of the amino acid sequence VLGDFGSSTKHVITAEE (SEQ ID NO:4); or iv) a CDON polypeptide consisting of the amino acid sequence KIRGKWLEHSTENY (SEQ ID NO:5).

The present disclosure also provides methods for inhibiting proliferation and inducing cell death in a population of cancer cells comprising administering to a subject in need of such treatment an effective amount of an anti-CDON antibody, or antigen-binding fragment thereof, that binds to CDON polypeptide, wherein the anti-CDON antibody, or antigen-binding fragment thereof, specifically binds an isolated peptide selected from: i) a CDON polypeptide consisting of amino acid residues 1 to 200 according to SEQ ID NO: 1, and ii) a CDON polypeptide consisting of amino acid residues 1000 to 1287 according to SEQ ID NO: 1.

The present disclosure also provides methods for inhibiting proliferation and inducing cell death in a population of cancer cells comprising administering to a subject in need of such treatment an effective amount of an anti-CDON antibody, or antigen-binding fragment thereof, that binds to CDON polypeptide, wherein the anti-CDON antibody, or antigen-binding fragment thereof, specifically binds an isolated peptide selected from: i) RVPESNPKAEVRYKIRGK (SEQ ID NO:2); ii) GIPLDSPTEVLQQPRET (SEQ ID NO:3); iii) VLGDFGSSTKHVITAEE (SEQ ID NO:4); or iv) KIRGKWLEHSTENY (SEQ ID NO:5).

The present disclosure also provides methods for inhibiting adhesion in a population of cancer cells comprising administering to a subject in need of such treatment an effective amount of an anti-CDON antibody, or antigen-binding fragment thereof, that binds to CDON polypeptide, wherein the anti-CDON antibody, or antigen-binding fragment thereof, specifically binds an isolated peptide selected from: i) a CDON polypeptide consisting of amino acid residues 1 to 200 according to SEQ ID NO: 1, and ii) a CDON polypeptide consisting of amino acid residues 1000 to 1287 according to SEQ ID NO:1.

The present disclosure also provides methods for inhibiting adhesion in a population of cancer cells comprising administering to a subject in need of such treatment an effective amount of an anti-CDON antibody, or antigen-binding fragment thereof, that binds to CDON polypeptide, wherein the anti-CDON antibody, or antigen-binding fragment thereof, specifically binds an isolated peptide selected from: i) RVPESNPKAEVRYKIRGK (SEQ ID NO:2); ii) GIPLDSPTEVLQQPRET (SEQ ID NO:3); iii) VLGDFGSSTKHVITAEE (SEQ ID NO:4); or iv) KIRGKWLEHSTENY (SEQ ID NO:5).

The present disclosure also provides an anti-CDON antibody, or antigen-binding fragment thereof, that specifically binds CDON polypeptide for use in a method of treating cancer.

The present disclosure also provides an anti-CDON antibody, or antigen-binding fragment thereof, that specifically binds to CDON polypeptide for use in the preparation of a medicament for treating cancer.

The present disclosure provides for use of an anti-CDON antibody, or antigen-binding fragment thereof, that specifically binds to CDON polypeptide in a method of treating cancer.

The present disclosure aso provides for use of an anti-CDON antibody, or antigen-binding fragment thereof, that specifically binds to CDON polypeptide in the preparation of a medicament for treating cancer.

As used herein, the terms “treat”, “treating”, or “treatment” and “prevent”, “preventing”, or “prevention” refer to eliciting the desired biological response, i.e., a therapeutic and prophylactic effect, respectively. In some embodiments, the therapeutic effect comprises one or more of a decrease/reduction in tumor, a decrease/reduction in the severity of the cancer (e.g., a reduction or inhibition of metastasis development), a decrease/reduction in symptoms and cancer-related effects, delaying the onset of symptoms and cancer-related effects, reducing the severity of symptoms and cancer-related effects, reducing the severity of an acute episode, reducing the number of symptoms and cancer-related effects, reducing the latency of symptoms and cancer-related effects, an amelioration of symptoms and cancer-related effects, reducing secondary symptoms, reducing secondary infections, preventing relapse to a disease, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and an increased survival time of the affected host animal, following administration of the anti-CDON antibodies, or antigen-binding fragments thereof, or compositions comprising the same. In some embodiments, a prophylactic effect may comprise a complete or partial avoidance/inhibition or a delay of cancer development/progression (e.g., a complete or partial avoidance/inhibition or a delay of metastasis development), and an increased survival time of the affected host animal, following administration of the anti-CDON antibodies, or antigen-binding fragments thereof, or compositions comprising the same.

The above parameters for assessing successful treatment and improvement in the disease are readily measurable by routine procedures familiar to a physician. For cancer therapy, efficacy can be measured, for example, by assessing the time to disease progression (TTP) and/or determining the response rate (RR). Metastasis can be determined by staging tests to determine the extent of metastasis. CT scans can also be carried out to look for spread to regions outside of the tumor or cancer. In some embodiments, the methods of prognosing, diagnosing and/or treating involves the determination and evaluation of CDON and/or hedgehog amplification and expression.

In some embodiments, administration of the anti-CDON antibodies, or antigen-binding fragments thereof, or compositions comprising the same, can be repeated, e.g., after one day, two days, three days, five days, one week, two weeks, three weeks, one month, five weeks, six weeks, seven weeks, eight weeks, two months or three months. The repeated administration can be at the same dose or at a different dose. The administration can be repeated once, twice, three times, four times, five times, six times, seven times, eight times, nine times, ten times, or more. For example, according to certain dosage regimens a patient receives anti-CDON therapy for a prolonged period of time, e.g., 6 months, 1 year or more. The amount of the anti-CDON antibodies, or antigen-binding fragments thereof, administered to the patient is in some embodiments a therapeutically effective amount. As used herein, a therapeutically effective amount or effective amount of anti-CDON antibodies, or antigen-binding fragments thereof, can be administered as a single dose or over the course of a therapeutic regimen, e.g., over the course of a week, two weeks, three weeks, one month, three months, six months, one year, or longer. Exemplary therapeutic regimens are further described below.

An “effective amount” of anti-CDON antibodies, or antigen-binding fragments thereof, is an amount sufficient to inhibit, partially or entirely, CDON activity. Alternately, an effective amount of anti-CDON antibodies, or antigen-binding fragments thereof, is an amount sufficient to reduce the rate of proliferation of a cancer cell and/or rate of survival of a cancer cell. An “effective amount” may be determined empirically and in a routine manner, in relation to this purpose.

A “therapeutically effective amount” refers to an anti-CDON antibody, or antigen-binding fragment thereof, or other drug effective to “treat” a disease or disorder in a subject or mammal. In some embodiments, the therapeutically effective amount of anti-CDON antibodies, or antigen-binding fragments thereof, will reduce the tumor size, inhibit (i.e., slow to some extent and preferably stop) the infiltration of tumor cells into peripheral tissue or organs, inhibit (i.e., slow to some extent and preferably stop) tumor metastasis, inhibit, to some extent, tumor growth, and/or relieve to some extent one or more of the symptoms associated with the tumor or cancer. To the extent the anti-CDON antibodies, or antigen-binding fragments thereof, may prevent growth and/or kill existing cancer cells, it may be cytostatic and/or cytotoxic.

Treatment of a cancer encompasses the treatment of patients already diagnosed as having any form of the cancer at any clinical stage or manifestation, the delay of the onset or evolution or aggravation or deterioration of the symptoms or signs of the cancer, and/or preventing and/or reducing the severity of the cancer.

A “subject” or “patient’ to whom the anti-CDON antibodies, or antigen-binding fragments thereof, is administered can be a mammal such as anon-primate (e.g., cow, pig, horse, cat, dog, rat, etc.) or a primate (e.g., monkey or human). In some embodiments, the subject or patient is a human. In some embodiments, the human is an adult patient. In some embodiments, the human is a pediatric patient.

A “therapeutic benefit” of anti-CDON antibodies, or antigen-binding fragments thereof, to treat cancer in a patient can result in any demonstrated clinical benefit compared with no therapy (when appropriate) or to a known standard of care. In some embodiments, clinical benefit is assessed based on objective response rate (ORR) (determined using RECIST version 1.1), duration of response (DOR), progression-free survival (PFS), and/or overall survival (OS). In some embodiments, a complete response indicates therapeutic benefit. In some embodiments, a partial response indicates therapeutic benefit. In some embodiments, stable disease indicates therapeutic benefit. In some embodiments, an increase in overall survival indicates therapeutic benefit. In some embodiments, therapeutic benefit may constitute an improvement in time to disease progression and/or an improvement in symptoms or quality of life. In some embodiments, therapeutic benefit may not translate to an increased period of disease control, but rather a markedly reduced symptom burden resulting in improved quality of life. As will be apparent to those of skill in the art, a therapeutic benefit may be observed using the anti-CDON antibodies, or antigen-binding fragments thereof, alone (monotherapy) or adjunctive to, or with, other anti-cancer therapies and/or targeted or non-targeted anti-cancer agents.

Typically, therapeutic benefit is assessed using standard clinical tests designed to measure the response to a new treatment for cancer. To assess the therapeutic benefits of the anti-CDON antibodies, or antigen-binding fragments thereof, one or a combination of the following tests can be used: 1) the Response Evaluation Criteria In Solid Tumors (RECIST) version 1.1,2) immune-related RECIST (irRECIST), 3) the Eastern Cooperative Oncology Group (ECOG) Performance Status, 4) immune-related response criteria (irRC), 5) disease evaluable by assessment of tumor antigens, 6) validated patient reported outcome scales, and/or 7) Kaplan-Meier estimates for overall survival and progression free survival.

Assessment of the change in tumor burden is a feature of the clinical evaluation of cancer therapeutics. Both tumor shrinkage (objective response) and time to the development of disease progression are endpoints in cancer clinical trials. Standardized response criteria, known as RECIST (Response Evaluation Criteria in Solid Tumors), were published in 2000. An update (RECIST 1.1) was released in 2009. RECIST criteria are typically used in clinical trials where objective response is the primary study endpoint, as well as in trials where assessment of stable disease, tumor progression or time to progression analyses are undertaken because these outcome measures are based on an assessment of anatomical tumor burden and its change over the course of the trial.

Additional criteria that may be used for clinical evaluation specific to cancer patients undergoing immune therapy treatment include the standardized immune-related RECIST (irRECIST) criteria (see, Nishino et al., Eur. J. Radiol., 2015, 84, 125 9-1268). These guidelines modified the RECIST 1.1 criteria above with consideration of potential immunomodulatory effects.

An exemplary therapeutic benefit resulting from the use of anti-CDON antibodies, or antigen-binding fragments thereof, to treat solid tumors, whether administered as monotherapy or adjunctive to, or with, other therapies or agents, is a complete response. Another exemplary therapeutic benefit resulting from the use of anti-CDON antibodies, or antigen-binding fragments thereof, to treat solid tumors, whether administered as monotherapy or adjunctive to, or with, other therapies or agents, is a partial response.

Validated patient reported outcome scales can also be used to denote response provided by each patient through a specific reporting system. Rather than being disease focused, such outcome scales are concerned with retained function while managing a chronic condition. A non-limiting example of a validated patient reported outcome scale is PROMIS® (Patient Reported Outcomes Measurement Information System) from the United States National Institutes of Health. For example, PROMIS® Physical Function Instrument for adult cancer patients can evaluate self-reported capabilities for the functioning of upper extremities (e.g., dexterity), lower extremities (e.g., walking or mobility), and central regions (e.g., neck, back mobility), and includes routine daily activities, such as running errands.

Kaplan-Meier curves (Kaplan and Meier, J. Am. Stat. Assoc., 1958, 53, 457-481) can also be used to estimate overall survival and progression free survival for cancer patients undergoing anti-CDON antibody therapy in comparison to standard of care.

The present disclosure also provides compositions comprising an anti-CDON antibody, or antigen-binding fragment thereof, and at least one pharmaceutically acceptable carrier and, optionally, one or more additional therapeutic agents, such as the combination therapeutic agents, described herein. The compositions will usually be supplied as part of a sterile, pharmaceutical composition that will normally include a pharmaceutically acceptable carrier. This composition can be in any suitable form, such as liquid form, in an aerosol, or in solid form (depending upon the desired method of administering to a patient).

Liquid forms include, but are not limited to, injectable solutions, aerosols, droplets, topological solutions, and oral suspensions. Exemplary solid forms include, but are not limited to, capsules, tablets, and controlled-release forms. The latter form is illustrated by miniosmotic pumps and implants. Other solid forms include, but are not limited to, creams, pastes, other topological applications, and the like.

The anti-CDON antibodies, or antigen-binding fragments thereof, can be administered to a patient by a variety of routes such as orally, transdermally, subcutaneously, intranasally, intravenously, intraarterially, intramuscularly, intraocularly, topically, locally, intrathecally, intracerebroventricularly, intraspinally, and inracranially. The most suitable route for administration in any given case will depend on the particular antibody, the subject, and the nature and severity of the disease and the physical condition of the subject. In some embodiments, the anti-CDON antibodies, or antigen-binding fragments thereof, can be formulated as an aqueous solution and administered by subcutaneous injection.

Pharmaceutical compositions can be conveniently presented in unit dose forms containing a predetermined amount of anti-CDON antibodies, or antigen-binding fragments thereof, per dose. Such a unit dose can contain for example, about 0.1 mg to about 5 g, about 1 mg to about 1 g, or bout 10 to about 50 mg. Pharmaceutically acceptable carriers for use in the disclosure can take a wide variety of forms depending, e.g., on the condition to be treated or route of administration.

Therapeutic formulations of the anti-CDON antibodies, or antigen-binding fragments thereof, can be prepared for storage as lyophilized formulations or aqueous solutions by mixing the anti-CDON antibodies, or antigen-binding fragments thereof, having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, e.g., Remington's Pharmaceutical Sciences, 16th edition (Osol, ed. 1980). Such additives are suitably nontoxic to the recipients at the dosages and concentrations employed.

The compositions may also contain buffering agents to maintain the pH in the range that approximates physiological conditions. The buffering agents can be present at concentrations ranging from about 2 mM to about 50 mM. Suitable buffering agents for use in the compositions include both organic and inorganic acids and salts thereof such as citrate buffers (e.g., monosodium citrate-disodium citrate mixture, citric acid-trisodium citrate mixture, citric acid-monos odium citrate mixture, etc.), succinate buffers (e.g., succinic acid monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives can be added to the compositions to retard microbial growth, and can be added in amounts ranging from about 0.2% to about 1% (w/v). Suitable preservatives for use include, but are not limited to, phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers, sometimes known as “stabilizers”, can be added to ensure isotonicity of liquid compositions and include, but are not limited to, polhydric sugar alcohols, for example, trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylie polymers, such as polyvinylpyrrolidone monosaccharides, such as xylose, mannose, fructose, glucose; disaccharides such as lactose, maltose, sucrose and trisaccacharides such as raffinose; and polysaccharides such as dextran. Stabilizers can be present in an amount from about 0.1 to about 10,000 weights per part of weight active protein.

Non-ionic surfactants or detergents (also known as “wetting agents’) can be added to the compositions to solubilize the anti-CDON antibodies, or antigen-binding fragments thereof, as well as to protect the anti-CDON antibodies, or antigen-binding fragments thereof, against agitation-induced aggregation, which also permits the formulation to be exposed to shear surface stressed without causing denaturation of the protein. Suitable non-ionic surfactants include, but are not limited to, polysorbates (20, 80, etc.), polyoxamers (184, 188 etc.), Pluronic polyols, polyoxyethylene sorbitan monoethers (TWEEN-20, TWEEN-80, etc.). Nonionic surfactants can be present in an amount from about 0.05 mg/mL to about 1.0 mg/mL, or from about 0.07 mg/mL to about 0.2 mg/mL.

Additional miscellaneous excipients that can be added to a composition include bulking agents (e.g., starch), chelating agents (e.g., EDTA), antioxidants (e.g., ascorbic acid, methionine, vitamin E), and cosolvents.

In some embodiments, the anti-CDON antibodies, or antigen-binding fragments thereof, may be encapsulated in liposomes. In some embodiments, the anti-CDON antibodies, or antigen-binding fragments thereof, may be encapsulated in polymer microspheres. Microspheres can be prepared from degradable polymers such as poly(lactide-co-glycolide) (PLG), polyanhydrides, poly (ortho esters), nonbiodegradable ethylvinyl acetate polymers, in which proteins are entrapped in the polymer. Polyethylene glycol (PEG)-coated nanospheres can also provide carriers for intravenous administration of therapeutic proteins.

The compositions described herein can also contain a combination therapeutic agent in addition to the anti-CDON antibodies, or antigen-binding fragments thereof. Examples of suitable combination therapeutic agents are provided herein.

The dosage of anti-CDON antibodies, or antigen-binding fragments thereof, to be administered will vary according to the particular antibody, the type of disease, the subject, and the severity of the disease, the physical condition of the subject, the therapeutic regimen (e.g., whether a combination therapeutic agent is used), and the selected route of administration. The appropriate dosage can be readily determined by a person skilled in the art.

It will be recognized by one of skill in the art that the optimal quantity and spacing of individual dosages of anti-CDON antibodies, or antigen-binding fragments thereof, will be determined by the nature and extent of the condition being treated, the form, route and site of administration, and the age and condition of the particular subject being treated, and that a physician will ultimately determine appropriate dosages to be used. This dosage can be repeated as often as appropriate. If side effects develop, the amount and/or frequency of the dosage can be altered or reduced, in accordance with normal clinical practice.

Although the pharmaceutical compositions provided herein are principally directed to pharmaceutical compositions which are suitable for administration to humans, it will be understood by the skilled artisan that such compositions are generally suitable for administration to animals of all sorts. Modification of pharmaceutical composition suitable for administration to humans in order to render the compositions suitable for administration to various animals is well understood, and the ordinarily skilled veterinary pharmacologist can design and perform such modification with little, if any, experimentation. Subjects to which administration of the pharmaceutical compositions described herein is contemplated include, but are not limited to, humans and other primates, and other mammals.

Described herein are combinatorial methods in which the anti-CDON antibodies, or antigen-binding fragments thereof, can be utilized. The combinatorial methods of the disclosure involve the administration of at least two agents to a patient, the first of which is an anti-CDON antibody, or antigen-binding fragment thereof, and the second of which is a combination therapeutic agent. The anti-CDON antibody, or antigen-binding fragment thereof, and the combination therapeutic agent can be administered simultaneously, sequentially or separately. The combinatorial therapy methods of the present disclosure can result in a greater than additive effect, providing therapeutic benefits where neither the anti-CDON antibodies, or antigen-binding fragments thereof, or combination therapeutic agent administered in an amount that is alone therapeutically effective.

The anti-CDON antibodies, or antigen-binding fragments thereof, and the combination therapeutic agent can be administered concurrently, either simultaneously or successively. The anti-CDON antibodies, or antigen-binding fragments thereof, and the combination therapeutic agent can be administered successively if they are administered to the patient on the same day, for example, during the same patient visit. Successive administration can occur 1, 2, 3, 4, 5, 6, 7 or 8 hours apart. In contrast, the anti-CDON antibodies, or antigen-binding fragments thereof, and the combination therapeutic agent can be administered separately if they are administered to the patient on different days, for example, anti-CDON antibodies, or antigen-binding fragments thereof, and the combination therapeutic agent can be administered at a 1-day, 2-day or 3-day, one-week, 2-week or monthly intervals. In some embodiments, administration of the anti-CDON antibodies, or antigen-binding fragments thereof, can precede or follow administration of the combination therapeutic agent.

As a non-limiting example, the anti-CDON antibodies, or antigen-binding fragments thereof, and combination therapeutic agent can be administered concurrently for a period of time, followed by a second period of time in which the administration of the anti-CDON antibodies, or antigen-binding fragments thereof, and the combination therapeutic agent is alternated.

In some embodiments, the combination therapeutic agent is a chemotherapeutic agent, an anti-angiogenic agent, an anti-rheumatic drug, an anti-inflammatory agent, a radiotherapeutic, an immunosuppressive agent, or a cytotoxic drug.

It is contemplated that when used to treat various diseases, the anti-CDON antibodies, or antigen-binding fragments thereof, can be combined with other therapeutic agents suitable for the same or similar diseases. When used for treating cancer, anti-CDON antibodies, or antigen-binding fragments thereof, may be used in combination with conventional cancer therapies, such as surgery, radiotherapy, chemotherapy or combinations thereof.

In some embodiments, other therapeutic agents useful for combination tumor therapy with the anti-CDON antibodies, or antigen-binding fragments thereof, include antagonists, e.g., antibodies, of other factors that are involved in tumor growth, such as HER2, HER3, HER4, VEGF, or TNF-α.

In some embodiments, for treatment of cancers, it may be beneficial to also administer one or more cytokines to the patient. Examples of cytokines include, but are not limited to, lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1 alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL).

In some embodiments, the anti-CDON antibody, or antigen-binding fragment thereof, is co-administered with a growth inhibitory agent. Suitable dosages for the growth inhibitory agent are those presently used and may be lowered due to the combined action (synergy) of the growth inhibitory agent and anti-CDON antibodies, or antigen-binding fragments thereof.

For treatment of cancers, anti-inflammatory agents can suitably be used in combination with the anti-CDON antibodies, or antigen-binding fragments thereof. Anti-inflammatory agents include, but are not limited to, acetaminophen, diphenhydramine, meperidine, dexamethasone, pentasa, mesalazine, asacol, codeine phosphate, benorylate, fenbufen, naprosyn, diclofenac, etodolac and indomethacin, aspirin, and ibuprofen.

For treatment of cancers, chemotherapeutic agents can suitably be used in combination with the anti-CDON antibodies, or antigen-binding fragments thereof. Chemotherapeutic agents include, but are not limited to, radioactive molecules, toxins, also referred to as cytotoxins or cytotoxic agents, which includes any agent that is detrimental to the viability of cells, agents, and liposomes or other vesicles containing chemotherapeutic compounds. Examples of suitable chemotherapeutic agents include, but are not limited to, 1-dehydrotestosterone, 5-fluorouracil decarbazine, 6-mercaptopurine, 6-thioguanine, actinomycin D, adriamycin, aldesleukin, an anti-α5β1 integrin antibody, alkylating agents, allopurinol sodium, altretamine, amifostine, anastrozole, anthramycin (AMC)), anti-mitotic agents, cisdichlorodiamine platinum (II) (DDP) cisplatin, diamino dichloro platinum, anthracyclines, antibiotics, antimetabolites, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), chlorambucil, cisplatin, cladribine, colchicin, conjugated estrogens, cyclophosphamide, cyclothosphamide, cytarabine, cytarabine, cytochalasin B, cytoxan, dacarbazine, dactinomycin, dactinomycin (formerly actinomycin), daunirubicin HCL, daunorucbicin citrate, denileukin diftitox, dexrazoxane, dibromomannitol, dihydroxy anthracin dione, docetaxel, dolasetron mesylate, doxorubicin HCL, dronabinol, E. coli L-asparaginase, eolociximab, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCL, glucocorticoids, goserelin acetate, gramicidin D, granisetron HCL, hydroxyurea, idarubicin HCL, Ifosfamide, interferon α-2b, irinotecan HCL, letrozole, leucovorin calcium, leuprolide acetate, levamisole HCL, lidocaine, lomustine, maytansinoid, mechlor ethamine HCL, medroxyprogesterone acetate, megestrol acetate, melphalan HCL, mercaptipurine, mesna, methotrexate, methyltestosterone, mithramycin, mitomycin C, mitotane, mitoxantrone, nilutamide, octreotide acetate, ondansetron HCL, paclitaxel, pamidronate disodium, pentostatin, pilocarpine HCL, plimycin, polifeprosan 20 with carmustine implant, porfimer sodium, procaine, procarbazine HCL, propranolol, rituximab, sargramostim, streptozotocin, tamoxifen, taxol, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

Any anti-angiogenic agent can be used in conjunction with the anti-CDON antibodies, or antigen-binding fragments thereof, including those listed by Carmeliet and Jain, Nature, 2000, 407, 249-257. In some embodiments, the anti-angiogenic agent is a VEGF antagonist or another VEGF receptor antagonist such as VEGF variants, soluble VEGF receptor fragments, aptamers capable of blocking VEGF or VEGFR, neutralizing anti-VEGFR antibodies, low molecule weight inhibitors of VEGFR tyrosine kinases and any combinations thereof. Alternately, or in addition, an anti-VEGF antibody may be co-administered to the patient.

The present disclosure also provides therapeutic regimens comprising administration of the anti-CDON antibodies, or antigen-binding fragments thereof. The therapeutic regimen will vary depending on the patient's age, weight, and disease condition. The therapeutic regimen can continue for 2 weeks to indefinitely. In some embodiments, the therapeutic regimen is continued from about 2 weeks to about 6 months, from about 3 months to about 5 years, from about 6 months to about 1 or about 2 years, from about 8 months to about 18 months, or the like. The therapeutic regimen can be a non-variable dose regimen or a multiple-variable dose regimen.

The amount of anti-CDON antibodies, or antigen-binding fragments thereof, administered will depend upon a variety of factors, including but not limited to, the particular type of solid tumor treated, the stage of the solid tumor being treated, the mode of administration, the frequency of administration, the desired therapeutic benefit, and other parameters such as the age, weight and other characteristics of the patient, etc. Determination of dosages effective to provide therapeutic benefit for specific modes and frequency of administration is within the capabilities of those skilled in the art.

In some embodiments, the anti-CDON antibodies, or antigen-binding fragments thereof, are provided as a lyophilized powder in a vial. The vials may contain abut 100 mg, about 110 mg, about 120 mg, about 150 mg, about 200 mg, about 250 mg, about 300 mg, or about 400 mg of the anti-CDON antibodies, or antigen-binding fragments thereof. Prior to administration, the lyophilized powder cn be reconstituted with sterile water for injection (SWFI) or other suitable medium to provide a solution containing about 20 mg/mL anti-CDON antibody, or antigen-binding fragment thereof. In some embodiments, the resulting reconstituted solution is further diluted with saline or other suitable medium for infusion and administered via an IV infusion twice every 7 days, once every 7 days, once every 14 days, once every 21 days, once every 28 days, once every 35 days, once every 42 days, once every 49 days, or once every 56 days. In some embodiments, for the first cycle, the infusion occurs over about 90 minutes. In some embodiments, subsequent infusions are over about 60 minutes.

In some embodiments, the anti-CDON antibodies, or antigen-binding fragments thereof, are administered as an IV infusion once every 7 days at about 0.1 mg/kg, about 0.5 mg/kg, about 1.0 mg/kg, about 2.0 mg/kg, about 3.0 mg/kg, about 4.0 mg/kg, about 5.0 mg/kg, about 6.0 mg/kg, about 8.0 mg/kg, or about 10.0 mg/kg. In some embodiments, the anti-CDON antibodies, or antigen-binding fragments thereof, are administered as an IV infusion once every 14 days at about 0.1 mg/kg, about 0.5 mg/kg, about 1.0 mg/kg, about 2.0 mg/kg, about 3.0 mg/kg, about 4.0 mg/kg, about 5.0 mg/kg, about 6.0 mg/kg, about 8.0 mg/kg, or about 10.0 mg/kg. In some embodiments, the anti-CDON antibodies, or antigen-binding fragments thereof, are administered as an IV infusion once every 21 days at about 0.1 mg/kg, about 0.5 mg/kg, about 1.0 mg/kg, about 2.0 mg/kg, about 3.0 mg/kg, about 4.0 mg/kg, about 5.0 mg/kg, about 6.0 mg/kg, about 8.0 mg/kg, or about 10.0 mg/kg. In some embodiments, the anti-CDON antibodies, or antigen-binding fragments thereof, are administered as an IV infusion once every 28 days at about 0.1 mg/kg, about 0.5 mg/kg, about 1.0 mg/kg, about 2.0 mg/kg, about 3.0 mg/kg, about 4.0 mg/kg, about 5.0 mg/kg, about 6.0 mg/kg, about 8.0 mg/kg, or about 10.0 mg/kg.

When administered adjunctive to or with other agents, such as other chemotherapeutic agents, the anti-CDON antibodies, or antigen-binding fragments thereof, may be administered on the same schedule as the other agent(s), or on a different schedule. When administered on the same schedule, the anti-CDON antibodies, or antigen-binding fragments thereof, may be administered before, after, or concurrently with the other agent. In some embodiments, where anti-CDON antibodies, or antigen-binding fragments thereof, are administered adjunctive to, or with, standards of care, the anti-CDON antibodies, or antigen-binding fragments thereof, may be initiated prior to commencement of the standard therapy, for example a day, several days, a week, several weeks, a month, or even several months before commencement of standard of care therapy. In some embodiments, where anti-CDON antibodies, or antigen-binding fragments thereof, are administered adjunctive to, or with, standards of care, the anti-CDON antibodies, or antigen-binding fragments thereof, may be initiated after commencement of the standard therapy, for example a day, several days, a week, several weeks, a month, or even several months after commencement of standard of care therapy.

As will be appreciated by those of skill in the art, the recommended dosages for the various agents described above may need to be adjusted to reflect patient response and maximize therapeutic benefit.

The present disclosure also provides pharmaceutical kits containing the anti-CDON antibodies, or antigen-binding fragments thereof, (including conjugates). In some embodiments, the pharmaceutical kit is a package comprising the anti-CDON antibodies, or antigen-binding fragments thereof (e.g., either in lyophilized form or as an aqueous solution), and one or more of the following: a combination therapeutic agent, a device for administering the anti-CDON antibodies, or antigen-binding fragments thereof, such as an injection pen, needle and/or syringe, and pharmaceutical grade water or buffer to re-suspend the anti-CDON antibodies, or antigen-binding fragments thereof, if the anti-CDON antibodies, or antigen-binding fragments thereof, are in lyophilized form.

In some embodiments, each unit dose of the anti-CDON antibodies, or antigen-binding fragments thereof, is packaged separately, and a kit can contain one or more unit doses (e.g., two unit doses, three unit doses, four unit doses, five unit doses, eight unit doses, ten unit doses, or more). In some embodiments, the one or more unit doses are each contained within a syringe or pen.

Diagnostic kits containing the anti-CDON antibodies, or antigen-binding fragments thereof (including conjugates), are also encompassed herein. In some embodiments, the diagnostic kit is a package comprising the anti-CDON antibodies, or antigen-binding fragments thereof (e.g., either in lyophilized form or as an aqueous solution), and one or more reagents useful for performing a diagnostic assay. Where the anti-CDON antibodies, or antigen-binding fragments thereof, are labeled with an enzyme, the kit can include substrates and cofactors required by the enzyme (e.g., a substrate precursor which provides the detectable chromophore or fluorophore). In addition, other additives can be included, such as stabilizers, buffers (e.g., a block buffer or lysis buffer), and the like. In some embodiments, the anti-CDON antibodies, or antigen-binding fragments thereof, included in a diagnostic kit are immobilized on a solid surface, or a solid support on which the anti-CDON antibodies, or antigen-binding fragments thereof, can be immobilized is included in the kit. The relative amounts of the various reagents can be varied widely to provide for concentrations in solution of the reagents which substantially optimize the sensitivity of the assay. In some embodiments, the anti-CDON antibodies, or antigen-binding fragments thereof, and one or more reagents can be provided (individually or combined) as dry powders, usually lyophilized, including excipients which on dissolution will provide a reagent solution having the appropriate concentration. Examples of solid supports include those formed partially or entirely of glass (e.g., controlled pore glass), polysaccharides (e.g., agarose), polyacrylamides, polystyrene, polyvinyl alcohol and silicones. In some embodiments, depending on the context, the solid phase can comprise a well of an assay plate, or a purification column (e.g., an affinity chromatography column). Solid supports also include discontinuous solid phase of discrete particles, such as those described in U.S. Pat. No. 4,275,149.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1: Boi Acts Within Hedgehog (Hh) Producing Cells to Control Hh Sequestration and Release in Response to Dietary Cholesterol in Drosophila

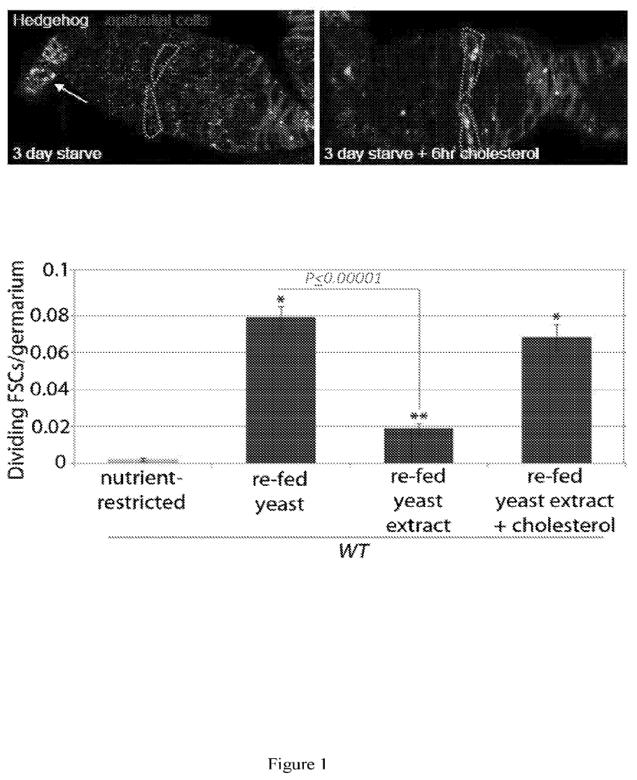

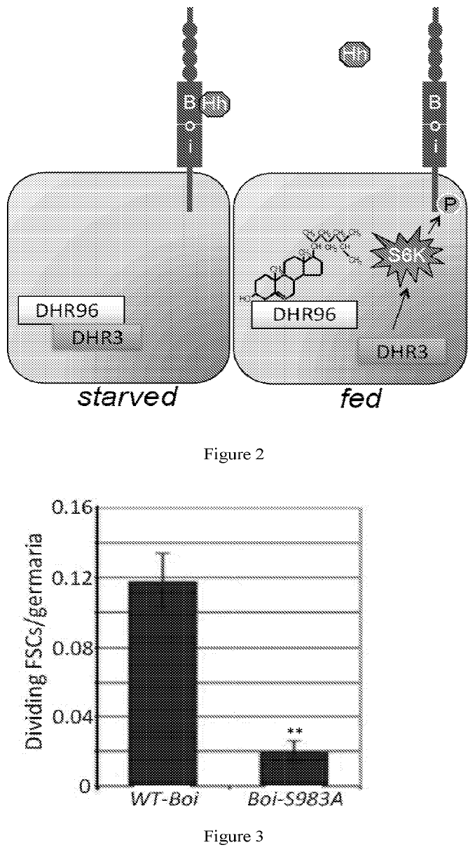

A novel mechanism that controls Hh levels in tissues has recently been identified. In Drosophila, follicle stem cells (FSCs) generate the follicular epithelium that supports egg development. Proliferation of FSCs was arrested in nutrient-restrictive conditions, but rapidly and robustly activated upon feeding (FIG. 1). It was found that Hh protein was sequestered on the surface of Hh producing cells in starved flies by direct association with its transmembrane receptor Boi and was released within 15 minutes after feeding. Hh ligand then accumulated in FSCs within 3-6 hours where it stimulated stem cell proliferation. This release mechanism depends on the presence of cholesterol, as cholesterol-free food was insufficient to trigger Hh release (FIG. 1). Additional data showed that ingestion of dietary cholesterol, but not carbohydrates or insulin, triggered Hh release via a novel inside-out signal transduction mechanism (FIG. 2). FSCs failed to proliferate 6 hours after feeding when expressing Boi with S983 mutated to alanine (BoiS983 A, FIG. 3). In addition, reducing S6K expression in the Hh producing cells suppressed FSC proliferation upon re-feeding, and activated forms of S6K were sufficient to drive FSC proliferation in starved flies. Cholesterol modification of Hh was dispensable for signaling in this system. Instead, cholesterol acted as a signal transduction molecule, binding directly to the steroid hormone receptor DHR96 and triggering the S6K-dependent phosphorylation that leads to Hh release (FIG. 2). Thus, DHR96 acted within Hh producing cells to modulate Hh sequestration and release by sensing systemic levels of dietary cholesterol.

### Example 2: Cholesterol Stimulates SHH Release from Human PDAC Cells

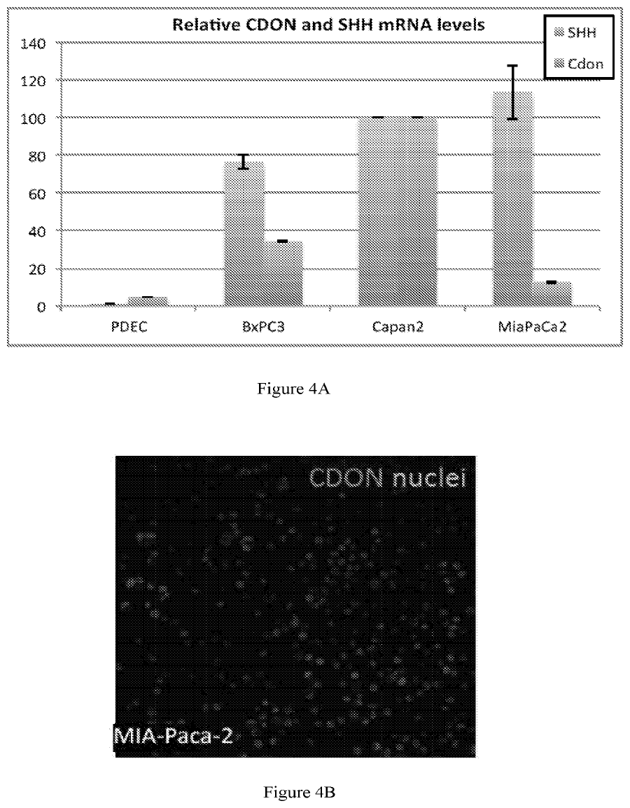

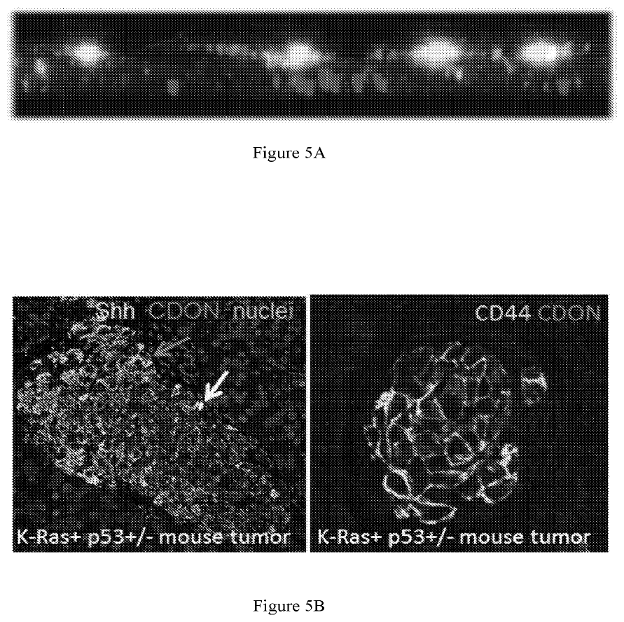

Based on the Drosophila results, it was hypothesized that CDON, the Drosophila homolog of Boi, would act to sequester SHH molecules in SHH producing cells that drive cancer progression. SHH is upregulated in pancreatic cancer, with expression detected in both early lesions (PanINs) and adenocarcinoma. It has been well established that SHH is expressed in and released from the pancreatic cancer cells, stimulating proliferation of a dense stroma that surrounds the tumor. This leads to formation of a barrier that both inhibits efficient drug delivery and restrains the spread of tumor cells. In addition, SHH signaling regulates pancreatic cancer stem-like cells, emphasizing the potential conservation of the mechanism that has been identified in the Drosophila. CDON has been shown to act as either a suppressor or enhancer of SHH signaling depending on whether it is expressed in SHH producing or receiving cells, respectively. Expression of the SHH pathway effector Smoothened (Smo) was undetectable in PDAC cells producing high levels of SHH, consistent with observations in the Drosophila that Hh producing cells lack Smo expression. If the Drosophila mechanism is conserved in PDAC, then CDON expression is predicted to be elevated in SHH-producing tumor cells to modulate the levels of SHH released. Consistent with this hypothesis, human PDAC cell lines Capan-2, BxPC3 and MIA PaCa-2 expressed CDON and SHH mRNA at high levels relative to the normal pancreatic epithelial cell line PDEC-hTERT (FIGS. 4A and 4B), with the two proteins co-localized on the apical surface (FIG. 5A). On the other hand, only a small number (0.2%) of cells in human PDAC cell lines exhibited high levels of SHH and/or CDON protein, with a similar ratio of CDON⁺ cells observed in PDACs derived from a mutant KRas+; p53−/+ mouse model (FIG. 5B).

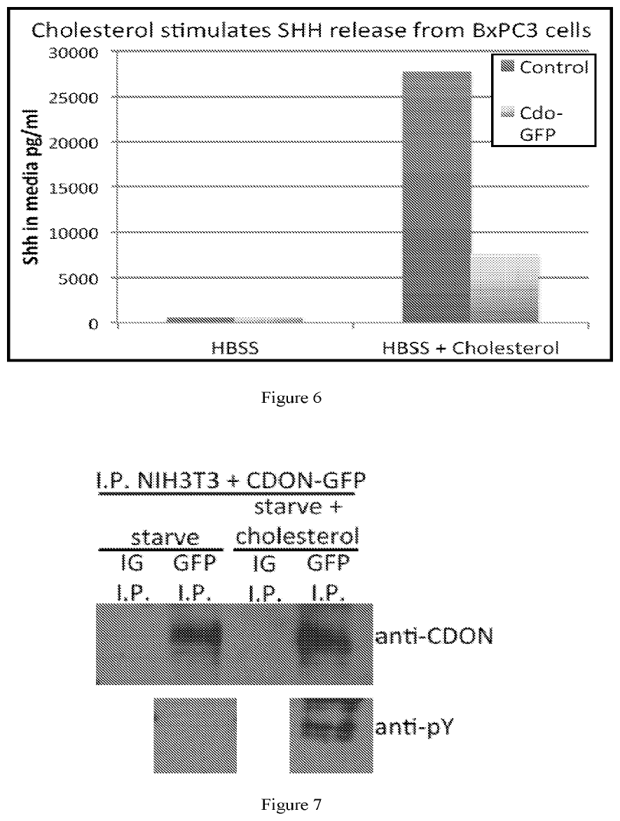

Like the Drosophila, starvation promoted SHH sequestration in BxPC3, Capan-2 and MIA PaCa-2 PDAC cells, with addition of cholesterol triggering SHH release (FIG. 6 and data not shown). Overexpression of wild-type CDON in the cells diminished the release of SHH after the addition of cholesterol, indicating that excess CDON can sequester SHH ligand (FIG. 6). CDON protein was phosphorylated in starved cells that have been stimulated with cholesterol (FIG. 7), although the modification differed from the Boi mechanism as CDON lacks an S6K target site and feeding-stimulated phosphorylation occurs on tyrosine rather than serine. These preliminary results demonstrated conservation of key aspects of the Drosophila sequestration mechanism in PDAC cells. This suggested that CDON expression limits SHH levels to prevent progression from pre-neoplastic lesions to adenocarcinoma.

### Example 3: Enhancement of RORa Using a Drug Agonist Increases the Levels of SHH Released from Pancreatic Cancer Cells

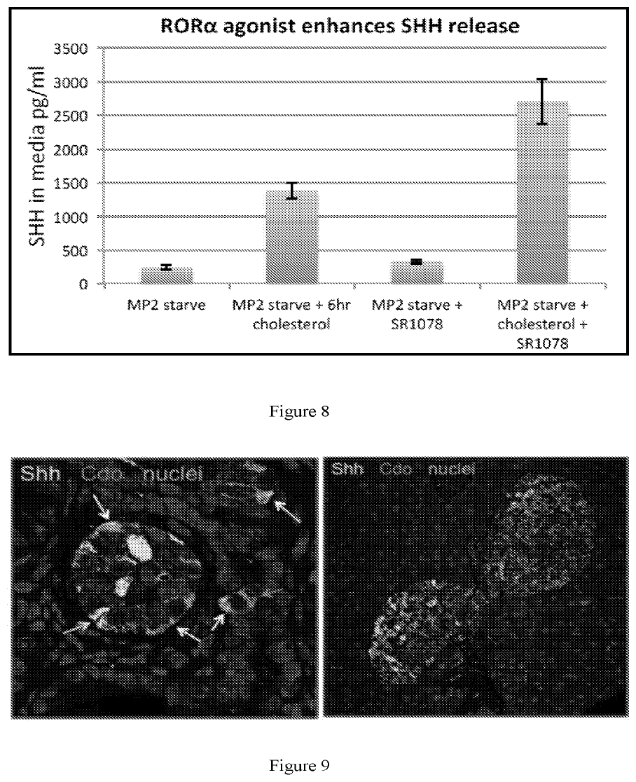

An interesting aspect of the Drosophila Hh release pathway is the requirement for two steroid hormone receptors in mediating the cholesterol signal (FIG. 2). If the human homologs of these receptors, Liver-X-Receptor (LXR) and Retinoic Orphan Receptor alpha (RORα) control SHH release in early pancreatic lesions, steroid-based drugs may have utility in controlling SHH release to prevent progression to PDAC. In a preliminary test, the activity of RORα was altered by treating the PDAC cell line MIA PaCa-2 with an RORα agonist (SR1078). SHH release was dramatically enhanced upon stimulation with SR1078 plus cholesterol relative to cholesterol stimulation alone, supporting a critical role of RORα in modulating SHH release (FIG. 8). The effects of cholesterol stimulation and RORα activation were suppressed by ectopic CDON expression (not shown), supporting a conserved role for CDON in modulating SHH release. These results demonstrated conservation of key aspects of the Hh sequestration and release pathway in PDAC and suggest: 1) a functional role for CDON in controlling SHH release in these cells, and 2) that RORα may be an effective target for drug-mediated control of SHH release in PDAC.

### Example 4: Murine Model of PDAC and Expression of Hedgehog Proteins

CDON expression is elevated in human pancreatic adenocarcinomas as compared to normal pancreatic tissue, which expresses no detectable CDON (see “world wide web” at “proteinatlas.org/ENSG00000064309-CDON/cancer”). To determine when aberrant CDON expression is initiated during PDAC development, CDON protein expression was examined in a genetic mouse model of pancreatic carcinoma. In this model, pancreatic carcinoma is initiated by activating mutations in K-Ras followed by loss-of-function of the tumor suppressor genes Trp53 or Cdkn2a. The genetically modified mouse model Pdx1-Cre LSL-K-RasG12D (KC model) expresses activated KRas (G12D) in the developing pancreas. The mice develop benign pancreatic lesions (PanINs) by 2-4 months, but pancreatic carcinoma develops in only 5-10% of mice after about 12 months. Loss of p53 in this background, by introduction of Trp53LoxP/LoxP (KPC model) accelerates tumor development to 9 weeks for pre-neoplastic lesions (90%), and PDAC-like lesions by 20 weeks (>80%). The consistent timeline of tumor development and progression in this model allows for rigorous analysis of the signaling and developmental events that occur at sequential stages of PDAC development. SHH expression was found in small groups of cells in PanINs that is maintained throughout tumor development (FIG. 9). Strikingly, CDON expression was detected as early as 9 weeks in small patches of SHH positive cells (FIG. 9). 5-10% of cells in PanINs express both CDON and SHH, supporting the hypothesis that CDON expression may act to suppress SHH signaling during tumor development.

### Example 5: CDON Expression in Human PDAC

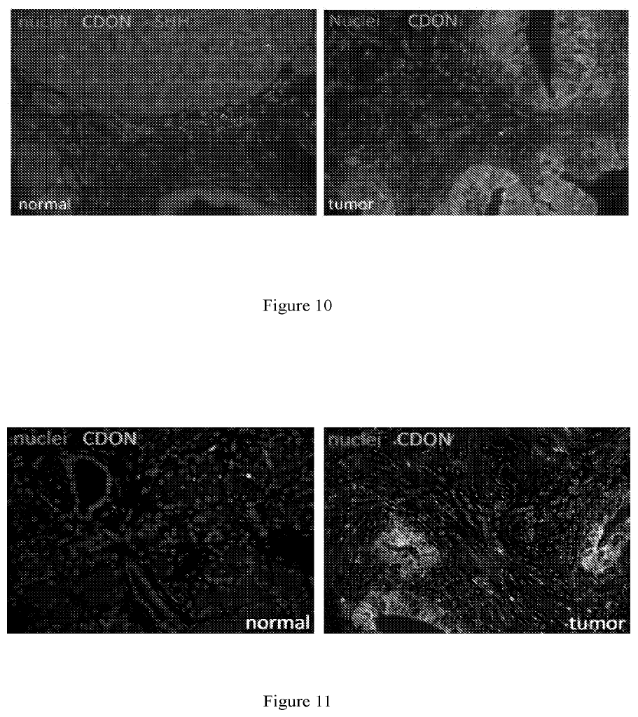

The preliminary data in PDAC cells and KPC mice suggests the possibility that a response of pancreatic epithelial cells to excess SHH is to induce expression of CDON in an attempt to control or restore normal signaling. Strikingly, in a pilot experiment on pancreatic tumor samples isolated from 13 patients, high expression CDON was found in PanINs and PDACs but undetectable in patient-matched normal pancreatic epithelial tissue, indicating that CDON expression may be an early marker of pancreatic adenocarcinoma (FIGS. 10 and 11).

### Example 6: Assessing the Effects of Loss of CDON Expression on Cholesterol-Stimulated SHH Release

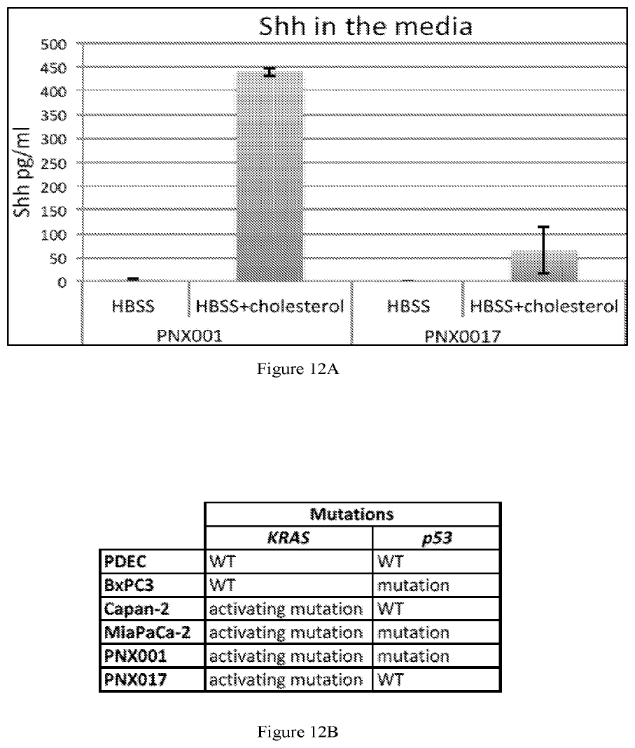

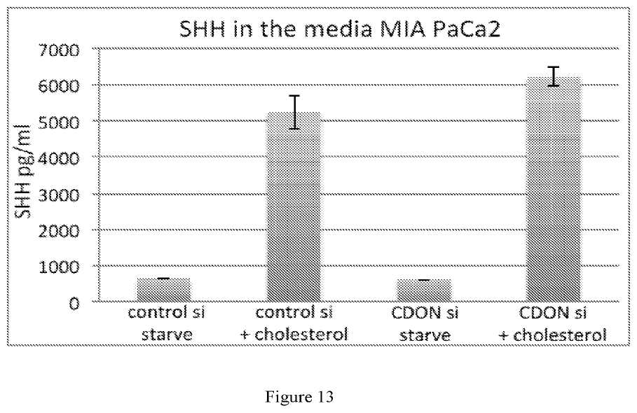

Pancreatic cancer cell lines BxPC3, MiaPaCa-2 and Capan-2 express high levels of CDON and SHH mRNA compared to normal pancreatic ductal epithelial cells (PDEC) (FIG. 4). Two deidentified patient-derived xenograft cell lines (PNX001 and PNX0017) that were recently established from tumors from PDAC patients were also tested. Similar to the BxPC3, MIA PaCa-2 and Capan-2 cells, these PDAC cell lines exhibit elevated expression of CDON and SHH mRNA (10- and 100-fold higher than PDEC, respectively). Capan-2, MIA PaCa-2, PNX001 and PNX0017 cells bear activating mutations of K-RAS, whereas BxPC3 cells represent the 5% of PDAC that lack mutations in K-RAS. All cell lines efficiently released SHH into the media after a period of starvation when cholesterol was introduced, indicating that cholesterol is a trigger for SHH release (FIG. 6 and FIGS. 12A and 12B). If CDON is required for SHH sequestration and release, then cells lacking CDON expression should constitutively release SHH into the media regardless of cholesterol levels. Alternately, if SHH cannot be released without first being sequestered by CDON on the surface of the cell, then loss of CDON will inhibit SHH release under any conditions. In an initial siRNA experiment, 80% knockdown of CDON RNA was achieved. This resulted in enhanced SHH release upon cholesterol stimulation, supporting the idea that a primary function of CDON is to limit SHH release (FIG. 13). However, the remaining 20% CDON expression and the observation that subsequent siRNA experiments were variable, with depletion levels averaging only 50%, suggest that the siRNA approach has significant limitations for fully defining the role of CDON in SHH release.

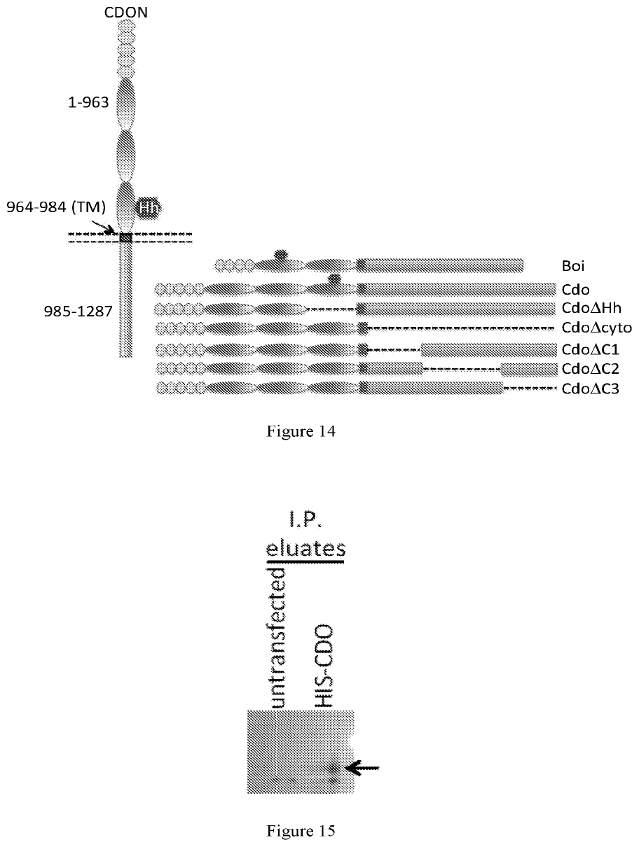

Major advances in genetic editing make it possible to directly test the hypothesis that CDON regulates cholesterol-dependent SHH in order to prevent tumorigenesis. An ideal system for this analysis should allow for stimulated elimination of the CDON gene and analysis of SHH release over time. The CDON locus will be edited using the PinPoint integrase system (System Biosciences). This is a multi-step process that enables the deletion of the wild-type CDON gene and leaves the option for targeted integration of wild-type or mutant forms of CDON into its endogenous locus. The first step involves Cas9-mediated incorporation of a PinPoint vector containing loxP sites that flank the endogenous CDON target region by homologous recombination. The cassette also includes attP recombination target sites that will be utilized to replace the cassette with an attB flanked cassette of choice, which will include a panel of CDON mutants (FIG. 14). Well-characterized pancreatic cancer cell lines BxPC3 and Capan-2 will be targeted initially, and results will be verified in patient derived xenograft cell lines PNX001 and PNX0017.

Newly generated BxPC3 and Capan-2 cell lines containing the incorporated loxP and attP sites will be treated with adeno-Cre to delete the CDON locus, and a timecourse of protein expression will be performed to determine when CDON RNA and protein expression are lost. After determining the optimal timepoint for CDON loss, the capacity of CDON-deleted cells to release SHH in response to cholesterol stimulation will be measured relative to normal CDON expressing cells as in FIG. 6. If CDON functions in the same manner as Boi does in the fly, constitutive SHH release in CDON null mutants will be observed.

### Example 7: The Domains of CDON that Regulate SHH Release in Response to Cholesterol

Once the effects of CDON deletion on cholesterol-stimulated SHH release are determined, the CDON functional domains required will be mapped in order to begin to identify upstream regulators of this event. Specific regions of the CDON protein have been mapped previously, including Ig domains at its N-terminus that mediate adhesion, three fibronectin repeats (one that binds SHH directly), a transmembrane domain, and a cytoplasmic tail that activates the CDON effectors p38MAPK, Akt, and CDC42 to control differentiation in muscle cells. PDAC cell lines bearing PinPoint insertions generated above will be utilized to target mutant forms of CDON into the endogenous locus. The previous insertion of an attP recombination target site in Capan-2 and BxPC3 cells will enable replacement of endogenous CDON with attB flanked mutant versions (FIG. 14). A donor vector including each mutant form of CDON will be introduced using PinPoint integrase, which catalyzes the reaction between the attP site in the endogenous CDON locus and an attB site in the donor vector, leading to insertion of the donor vector at the desired locus. The endogenous CDON gene will be excised in these cells by treatment with adeno-Cre, resulting in expression of only the mutant form of CDON in the resulting cells.

The mutants will be generated using CRISPR/Cas9 gene editing based PinPoint integrase system. Initial mutants will include a version of CDON lacking the first fibronectin domain that binds directly to SHH. This mutant is expected to lack the ability to sequester SHH. If the model is correct, SHH should be constitutively released in cells expressing this mutant. Conservation of the inside-out signaling model identified in the Drosophila will be tested by expressing a version of CDON lacking the cytoplasmic domain, which is predicted to sequester SHH, but lack the ability to release it in response to cholesterol stimulation. GFP will be fused to the C-terminus of these mutant forms of CDON so that cells expressing mutant CDON after recombination can be confirmed. SHH sequestration and release will be measured in cells expressing mutant forms of CDON during periods of starvation and cholesterol feeding. Based on previous work in the Drosophila, the SHH binding domain is predicted to be necessary for SHH sequestration, and the cytoplasmic domain required for its release.

Mutants of the Ig domain (Δlg), the fibronectin domains (ΔFN1/2 domain that mediates cadherin interaction, the ΔFN3 domain that is required for SHH binding) and the cytoplasmic domain (Δcyto) that mediates downstream signaling will be constructed and used to transfect ovarian and/or pancreatic cell lines harboring the loxP flanked CDON allele (e.g., Kuramochi, CaOV3 and PDX OC-1 cells). Comparison of isogenic cells expressing wild type and individual CDON mutants will allow us to parse the structual requirements for induction of CDON protein expression and 3D growth. The consequences of expression of mutant forms of CDON will be evaluated using IF and confocal microscopy and assays for viability, apoptosis and multicellular spheroid formation as described above. If the Δcyto domain mutant results in abrogation of any of these functions, finer mapping of the key regions mediating functional changes will be mapped using mutants encoding the membrane proximal, central and c-terminal domain mutants (ΔC1, ΔC2 and ΔC3). All functional analyses will be conducted by comparing isogenic parental cell lines expressing wild type CDON as a control.

### Example 8: The Role of Phosphorylation of the CDON Cytoplasmic Domain in SHH Release in Response to Cholesterol

In Drosophila, Hh release is triggered upon phosphorylation of the cytoplasmic tail of Boi by S6-kinase. Despite the high degree of conservation in the extracellular domains of CDON and Boi (36-70% homology56), functional similarities in Hh signaling regulation, and SHH/Hh binding location in fibronectin III domains of each protein, some details differ between the two Hh receptors. First, the mechanism of direct binding of ligand to receptor is different, with heparin-dependent Hh binding to Boi and calcium-dependent SHH binding to CDON. Second, CDON lacks conservation of the S6K target site. Instead, 37 putative phosphorylation sites (20 serines, 12 threonines, and 5 tyrosines) are predicted in the CDON cytoplasmic domain. In a preliminary experiment, it was found that cholesterol stimulated tyrosine phosphorylation of CDON (FIG. 7), suggesting the possibility that an analogous kinase-dependent mechanism may promote SHH release from CDON as was previously observed for Boi.

To define the role of CDON phosphorylation in cholesterol-mediated SHHrelease, cholesterol-stimulated target phosphorylation sites in CDON will be identified. His-CDON will be immunoprecipitated from starved or cholesterol-fed MIA PaCa-2 cells and the samples will be subjected to mass spectrometry phospho-site analysis. Antibodies targeting individual CDON phospho-sites will be generated and utilized to determine the time course of phosphorylation upon cholesterol stimulation. In an initial experiment, CDON was robustly phosphorylated on tyrosine at 6 hours after cholesterol stimulation (FIG. 15), suggesting earlier induction of kinase activity. Once a timecourse of phosphorylation is established, the functional relevance of specific phosphorylation events on SHH release will be determined.

To assess the functional relevance, CDON isoforms will be created that contain mutation of target sites of CDON to A (for ser/thr kinases) or F (for pY kinases) or D/E to generate non-phosphorylateable and potentially constitutively activated versions. PDAC cell lines will be generated that express phospho-mutant and phosphomimetic versions of CDON by targeting the attP sites in cells generated using the Pinpoint Integrase System. These mutant forms of CDON will be assayed for release of SHH in response to cholesterol as described above. The prediction is that mutation to A or F will abrogate the release response and mutation to D/E may result in a version of CDON that cannot sequester SHH.

Once specific phosphorylation sites are identified and functional roles for the phosphorylation event in SHH release are demonstrated, known consensus sequences will be utilized to identify the kinase(s) responsible for the critical phosphorylation event(s). Two kinases have been shown previously to bind to CDON or transmit CDON-dependent signals, Abl and p38MAPK, which are important in CDON-dependent muscle differentiation. Additional predicted phosphorylation sites are associated with consensus sequences of other kinases, and small molecule inhibitors are commercially available for many of these candidate proteins. Once likely candidates are identified by simple sequence analysis, BxPC3 and Capan-2 cells expressing wild type CDON will be treated with specific small molecule inhibitors. Cells will be treated with inhibitor or DMSO for 48 hours before starving the cells overnight. Cells will continue to be treated with the drug or DMSO during starvation and after cholesterol stimulation. The activity levels of the targeted kinases will be analyzed in response to cholesterol in drug- or control-treated cells using western blot or activity assays (e.g. p38 MAPK Activity Assay kit (Sigma Aldrich)) at timepoints determined above, prior to the time when CDON phosphorylation is first detectable. Levels of SHH in the media will be analyzed by SHH ELISA to determine if blocking kinase activity inhibits SHH release.

Once the kinase(s) regulates SHH release is determined, targeted CRISPR/Cas9 will be performed to specifically reduce the kinase of interest in the BxPC3 and Capan-2 cells to verify specific requirements for individual kinases in SHH release and rule out off-target effects of the kinase inhibitors. Additionally, activated versions of the kinases will be expressed in the cells. The loss of the kinase target site will block SHH release when starved cells are stimulated with cholesterol, and that constitutively activated kinases will promote SHH release independently of cholesterol treatment.

### Example 9: The Role of LXR and RORα in CDON Expressing Cells and SHH Sequestration and Release

The steroid hormone receptors DHR96 and DHR3 are necessary for Hh release in the Drosophila, and their homologs LXR and RORα are expressed in PDAC cells. LXR is implicated in mediating cholesterol signaling in KRAS-dependent tumors, and treatment of PDAC cells with an agonist (SRI 0789) to the DHR3 homolog RORα enhanced cholesterol-dependent SHH release (FIG. 8). The enhancement of SHH release in cells treated with SRI 0789 was suppressed by CDON expression, supporting a likely role for RORα in modulating SHH release.

To define the roles of LXR and RORα in cholesterol-stimulated SHH release, SHH release will be analyzed in cells lacking expression of LXR or RORα. Initially expression of each of these targets will be knocked out. In a preliminary siRNA experiment, 80% knockdown of CDON mRNA resulted in enhanced SHH release upon cholesterol stimulation, supporting the notion that a primary function of CDON is to limit SHH release. However, siRNA experiments can give variable levels of mRNA depletion. To develop a more robust system for this analysis, the CDON, LXR and RORα loci in PDAC cells will be genetically altered using a lenti-viral CRISPR/Cas9 system. Three sgRNAs (short-guide RNAs) will be designed based on computer prediction software (crispr.mit.edu) to target each gene. The sgRNAs will be individually cloned into HF-lentiCRISPRv2 that expresses a high fidelity Cas9 protein when integrated into target cell genomes. sgRNA-lentiviruses will be produced from 293T packaging cell lines cotransfected with packaging plasmids psPAX2 and pCMV-VSVg. Well-characterized pancreatic cancer cell lines BxPC3 and Capan-2 will be transduced initially, and results verified in patient-derived cell lines PNX001 and PNX0017. Puromycin-resistant cells will be tested by western blot and qRT-PCR to confirm CDON, LXR or RORα loss. After verifying loss of CDON, LXR or RORα, the capacity of these cells to release SHH in response to cholesterol stimulation relative to wild type parental cells will be measured as in FIG. 6. If CDON functions in the same manner as Boi does in the Drosophila, constitutive SHH release in CDON null mutants, and abrogation of cholesterol-mediated SHH release in LXR or RORα null mutants is likely. If altered SHH release is observed in the LXR/RORα mutants, a major goal will be to develop drug treatments that achieve the same result. Small molecules that specifically promote or inhibit the activity of LXR and RORα are available, presenting an ideal opportunity to test the efficacy of these drugs in controlling SHH release. Loss of LXR or RORα expression is likely to abrogate cholesterol-mediated SHH release. Similarly, treatment with drug antagonists targeting these steroid hormone receptors should block SHH release and agonists enhance release.

As drugs that target LXR and RORα are fat soluble, they have potential utility in PDAC tumors that are resistant to chemotherapy due to lack of sufficient circulation-dependent delivery.

### Example 10: The Role of CDON and the Effects of Kinase and Steroid Hormone Inhibitors on Fibroblast Alterations Caused by PDAC

Once the SHH sequestration and release control mechanisms are defined, the cells expressing mutant versions of CDON will be utilized in a co-culture system to assess the effects of CDON-mediated sequestration and release of SHH on stromal induction. To verify that CDON inhibits SHH release and downstream activation of stromal cells, BxPC3 and Capan-2 cells expressing wild type or mutated CDON will be co-cultured with NIH 3T3 fibroblasts that stably express a Gli-responsive luciferase reporter and a constitutive Renilla-luciferase expression vector (SHH-Light II). Comparison of the ratio of Gli-luciferase to the SHH-independent Renilla-luciferase provides a quantitative measure of SHH pathway activity upon stimulation with cultured media from the genetically modified PDAC cell lines. These cells will be used to analyze SHH pathway activity via measurement of target gene activation. To determine if release of SHH via a cholesterol-induced CDON mechanism can stimulate SHH signaling in fibroblasts directly, NIH-3T3/GLI-luc fibroblasts will be seeded in the lower wells of a transwell cell culture system (6-well type, high-density membrane with 0.45 mm pores, BD Biosciences) and grown to 70-80% confluency. Capan-2, BxPC3, or the CDON-null version of these cell lines will then be seeded in the upper chambers and cultured in complete medium. After 24 hours in culture, PDAC cells in the upper chamber will be starved overnight in HBSS, and treated with HBSS+/− cholesterol 12 hours later. After a 12-hour incubation, cells will be lysed and SHH activation in the fibroblasts will be determined by measuring the luciferase to Renilla-luciferase ratio. Cells containing wildtype CDON are likely to sequester SHH in starved cells and release SHH to induce Gli-reporter activity in the fibroblasts after cholesterol exposure. Cells lacking CDON are likely to result in constitutive SHH activity in the fibroblasts due to the lack of SHH sequestration, even in the absence of cholesterol.

The effects of kinase and RORα inhibition will also be measured using wild type Capan-2 and BxPC3 cells. Cells will be grown in the transwell co-culture assay as described in the presence of identified kinase inhibitors, RORα agonist SR1078, or vehicle as described. SR1078 and any kinase inhibitors that block CDON phosphorylation are likely to block SHH-stimulated Gli-reporter activity in the fibroblasts even in the presence of cholesterol.

### Example 11: The Role of Cholesterol in Diet-Induced PDAC Initiation

To determine whether ingestion of cholesterol accelerates K-RAS initiated PanIN formation in an SHH/CDON-dependent manner, the effect of dietary cholesterol levels on CDON/SHH expression and localization will be measured at key developmental transition timepoints during PDAC progression. Simultaneously, the effects of a high cholesterol diet on tumor initiation and progression will be assessed.

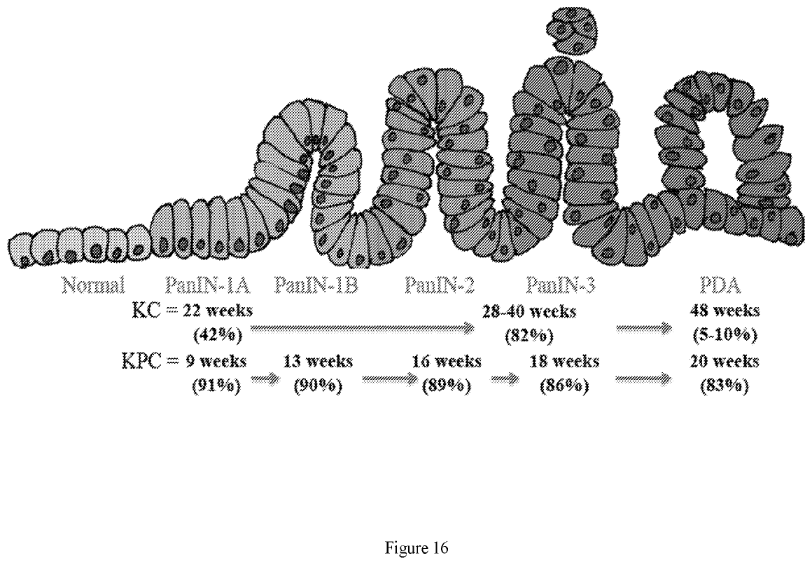

Treatment with statins to reduce serum cholesterol delays PDAC development in mice bearing activating mutations in KRAS and loss of p53 (Pdx1-Cre/LSL-KrasG12D/Trp53LoxP/+, KPC mice) in pancreatic epithelial cells. Moreover, KC mice (bearing an activating KRAS mutation) fed a high fat diet develop early pancreatic neoplasms rapidly relative to mice fed a normal, low-fat diet, although the molecular mechanisms that mediate this response are undefined. The initial approach will be to determine the expression and localization of CDON relative to SHH at well-defined timepoints that correlate with specific stages of tumor progression in KPC mice fed a normal diet containing no-cholesterol or a high (2%) cholesterol diet continuously after weaning (FIGS. 9 and 16). To confirm that the diet is effectively changing serum cholesterol levels, serum from mice will be tested for total cholesterol, HDL and LDL/VDL (Cholesterol Assay Kit, Abeam) at weaning and before sacrifice at the pre-determined timepoint.

The preliminary data suggests that CDON and SHH are expressed as early as 9 weeks after birth in KPC mice, in the first stage of PanIN development (PanIN-1 A, FIGS. 9 and 16). A comprehensive analysis will be performed to measure SHH and CDON expression levels and patterns at weeks 6, 8, 12, 16, and 20 weeks in mice from the two feeding groups (N=10 mice per timepoint). The analysis will also be performed in Pdx1-Cre LSL-K-RasG12D (KC mice), which express activated K-Ras (G12D) in the developing pancreas, but have normal levels of p53 function. KC mice develop pre-neoplastic pancreatic lesions (PanINs) with much longer latency relative to KPC mice, with initial lesions appearing by 2-4 months, and PDAC development in only 5-10% of mice after about 12 months. This relatively slow developmental model will enable accurate measurement of potential differences in the timing of SHH and CDON expression, as the stages of PDAC development will be spread over a longer time period.

To evaluate the effects of a high cholesterol diet on tumor progression, pancreatic tissue will be isolated at the timepoints indicated above. The evaluator will be blinded to the experimental groups and histologically evaluate twenty fields of each pancreas section from a single H&E slide per animal. PanIN lesions and adenocarcinoma will be classified according to published criteria. The total number of ductal lesions and their grade will be scored for all fields, and the relative proportion of each PanIN lesion grade to total number of ducts analyzed will be recorded. Scores will include no significant lesions (indicating normal appearance), acinar-ductal metaplasia, PanIN la, PanIN lb, PanIN 2, PanIN 3, early adenocarcinoma, and adenocarcinoma. Statistical differences between mice fed low or high cholesterol diets will be determined using Fisher's exact test for PDAC incidence and unpaired t-test with Welch's correction for PanINs and PDAC lesions. Differences between groups are considered significant at p<0.05.

Timing of expression of CDON and SHH will be analyzed based on staining from standard immunohistochemical detection protocols in sections adjacent to those scored for staging of the tissue. SHH and CDON expression will be measured at 6, 8, 12, 16, and 20 weeks of age in the KPC mice and at 16, 24, 32, 40, and 48 weeks of age in the KC mice to accurately determine: 1) the relative expression of the two proteins during PDAC development, and 2) the influence of a high cholesterol diet on their expression.

In addition to analysis of the relative initiation of SHH and CDON expression, the effects of the high cholesterol diet on induction of desmoplasia in stromal cells will be measured. Localization of CDO and SHH in combination with markers for tumor (cytokeratin) or stromal (α-SMA) cells will be analyzed and expression levels directly compared between tumor and stromal tissue. Immunohistological staining of each protein will be scored automatically using the Vectra Automated Multispectral Imaging System (Perkin Elmer), which accurately measures morphometric characteristics on whole slides or in distinct tissue regions of interest. The Vectra system can accurately measure protein expression in slides labeled with H&E, immunofluorescence and immunohistochemical stains in up to 200 slides in a single batch run. One or more proteins can be measured on a per tissue or per cell compartment, and in Form software will automatically quantitate data acquisition and extraction. Due to the ability to analyze multiple proteins in addition to tumor and stroma markers simultaneously, the CDON and SHH expression will be documented along with morphological changes in the tumor and stroma over time, and establish an unbiased timeline to determine the correlation of CDON and SHH expression and pancreatic lesion progression.

Once the raw measurements of: 1) incidence of pre-malignant or malignant lesions, 2) CDON and SHH expression, and 3) stromal activation are available, differences between mice fed low or high cholesterol diets will be assessed using statistical analysis. Correlations between protein expression timing and levels, dietary influence, and tumor progression will be determined. The specific tests used will depend on the data collected, and additional mice will be added to the study if more power is needed to conduct the calculations. For example, to have sufficient power to detect differences in proportions of mice having tumors at a given timepoint, for a small difference in rates (e.g. 20% difference), up to 36 mice per group may be required. If the differences in proportions are larger, fewer mice will be required. Differences will be evaluated using exact binomial tests.

CDON is likely to limit SHH levels at early timepoints to maintain a normal stroma and benign lesions in mice fed a low cholesterol diet. At later timepoints, the levels of SHH may overwhelm the ability of CDON to sequester SHH molecules, resulting in SHH-dependent induction of desmoplasia in surrounding stromal cells. Once the stroma is altered, tumor progression is likely to accelerate, resulting in the transition from pre-malignant lesions to PDAC. In addition, mice fed a high cholesterol diet are likely to exhibit stromal activation at earlier timepoints due to cholesterol-triggered SHH release from CDON. This will result in rapid induction of desmoplasia and accelerated tumor development relative to mice fed a low cholesterol diet.

### Example 12: The Role of CDON in Diet-Induced PDAC Initiation

To examine whether dietary cholesterol stimulates release of SHH from CDON, and contribute to tumor progression, how the lack of CDON in the KC mice affects the timing of K-RASinducible PanIN development will be assessed. These studies will focus on KC mice due to reduced genetic complexity and time to breed mice, in addition to the ability to more accurately pinpoint changes in progression from preneoplastic lesions in these mice with a much longer latency period. Mice lacking all CDON expression will be crossed into the KC background, resulting in CDON homozygous loss in the PDAC model. The initial approach will be to utilize a commercially available mouse strain bearing an insertion of beta-geo and IRES-PLAP between exons 13 and 14 of the CDON locus. The presence of a splice acceptor site and transcription stop/poly-adenylation signal promotes generation of a fusion protein containing most of the extracellular domain of CDON fused to β-Gal. This “knock-in” abolishes expression of the targeted CDON gene (B6.129P2-Cdontm1Aok/Mmucd, MMRRC, referred from here on out as Cdon^{null}). Cdon^{null} homozygous mutant mice are viable but have mild to moderate craniofacial midline defects due to disruption of SHH signaling during brain development.74 Cdon^{null} mice will be crossed with KC mice to create Cdon^{null}KC mice that have homozygous deletion of CDON and expression of activated KRAS in pancreatic precursor cells. Beta-galactosidase will be expressed in CDON-expressing tissues of Cdon^{null}KC mice.

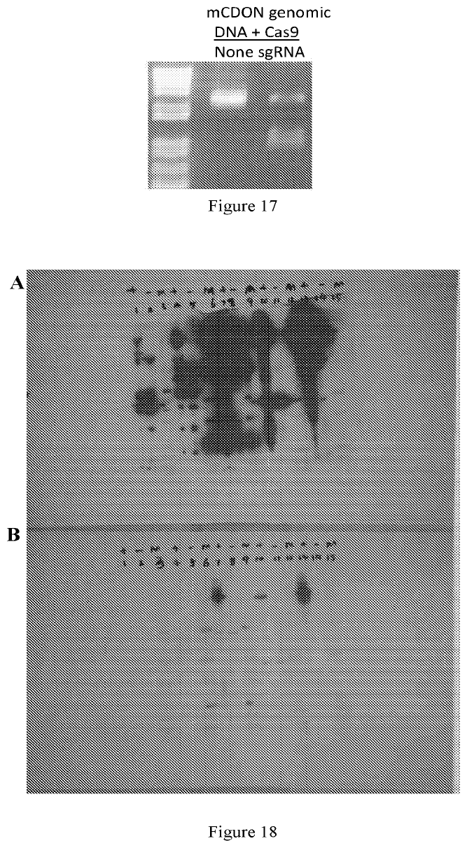

To address a potential for the reduced viability of the Cdon^{null} mice, a conditional CDON knockout mouse (Cdon^{fl/fl}) will be generated to enable CDON deletion only in pancreatic epithelial cells. LoxP sites will be inserted upstream of exon 13 and downstream of exon15 using CRISPR/Cas9 mediated homologous recombination. Four sgRNAs (short guide RNAs) efficiently directed Cas9-mediated cleavage at the genomic DNA site in vitro (FIG. 17). Mouse oocytes will be injected with: a) in vitro transcribed CDON sgRNA, b) a Cas9 expression plasmid, and c) a DNA construct containing left and right homology arms and an intermediate cassette including the loxP sites and Cdon genomic DNA sequences. Cre-mediated excision will result in deletion of exons 13-15 (which contain the SHH binding domain) and loss of the transmembrane and cytoplasmic domains. Cdon^{fl/fl} mice will be crossed with KC mice to generate Cdon^{fl/fl}KC mice that specifically delete CDON and activate K-RAS in pancreatic precursor cells. As above, Cdon^{fl/fl}KC and control littermates will be fed a normal no cholesterol diet and sacrificed at 16, 24, 32, 40, and 48 weeks of age for analysis of PanINs.

### Example 13: The Effect of RO Ra Agonist on PDAC Development in KPC Mice

Treatment of PDAC cells with RORα agonist SR1078, a critical component of the fly Hh release pathway, enhanced cholesteroldependent SHH release (FIG. 8). As drugs that target RORα are fat soluble, they have potential utility in PDAC tumors that are resistant to chemotherapy due to lack of sufficient drug delivery. Moreover, manipulation of SHH release may alter the ability of tumor cells to influence stromal alterations that promote tumorigenesis, reducing tumor burden and potency. To define the role of RORα on SHH release in vivo, KPC mice (which develop PanINs by 8-9 weeks and adenocarcinoma by 20 weeks of age) on a normal no cholesterol diet will be treated with RORα agonist SRI 078 (10 mg/kg) and sacrificed at 8, 14 and 20 weeks of age for analysis of PanIN and adenocarcinoma development. Initially 5 mice per timepoint will be used in a pilot experiment.

### Example 14: Identification of Novel Biomarkers of Pancreatic Cancer

In lung, breast and ovarian cancer, CDON has been identified as a prognostic marker based on publicly available RNA-Seq data. Previous work showed that expression of specific proteins in early precancerous lesions is predictive of risk for development of cancer. Thus, the expression levels of the CDON proteins studied herein in pancreatic lesions using pancreatic tissue microarrays (TMA) may be indicative of presence or progress of pancreatic cancer.

Through the BRF, a cohort of more than 160 PDAC cases have been identified that are currently available in 6 TMAs, and an additional 54 samples available for future TMAs. The arrays contain matched normal pancreas controls, intraductal papillary mucinous neoplasms, and a majority of invasive and metastatic PDACs. All clinical specimens are de-identified, with well-annotated clinical and pathological information available. This data indicates that the specimens used for the TMA are representative of populations that are typical for PDAC. Using existing TMAs, an optimized analysis system has been developed, where 6 independent proteins can be analyzed and expression levels directly compared between tumor and stromal tissue to correlate expression levels with clinical outcome. Using these established methods, expression levels and localization of CDON, SHH, LXR and RORα will be measured in combination with markers for epithelial (cyto-keratin) or stromal (α-SMA) cells in the described TMAs. Immunohistological staining of each protein will be scored automatically using the Vectra Multi Spectral Imaging System (Perkin Elmer) and customized programs written for this type of analyses (code available at the world wide web at “github.com/cukie”). The correlation between staining levels and clinical parameters such as metastasis and survival will be determined by univariate analysis using CART (Classification and Regression Trees methodology) to identify the prognostic potential of each protein. Tumor and serum samples from 96 patients have been matched and can be utilized in a pilot experiment to analyze correlations between serum cholesterol levels and protein expression. Statistical significance of correlations between expression levels and clinical outcomes will be calculated. This work will define links between expression of Hh pathway components during tumorigenesis and clinical outcome, as well as uncover markers for various well-defined stages of tumor development. Identification of novel proteins involved in the development of pancreatic cancer will potentially provide novel drug targets for treatment. CDON, a cell surface receptor, is a particularly intriguing candidate as receptors have shown potential for precision drug-conjugated targeting to tumor cells, an option particularly appealing for difficult to treat pancreatic cancer.

### Example 15: Making of a Polyclonal Anti-CDON Antibody

The antibodies were generated under a contract for antibody production with Thermo Fisher Scientific using a standard 70-day rabbit immunization protocol for rabbit polyclonal antibody production. Two rabbits were immunized with a polypeptide having an N-terminal CDON sequence RVPESNPKAEVRYKIRGK (amino acids 142-159, part of extracellular domain, SEQ ID NO:2). In addition, two rabbits were immunized with a polypeptide having an C-terminal CDON sequence GIPLDSPTEVLQQPRET (amino acids 1271-1287, part of cytoplasmic domain, SEQ ID NO:3). On day 0, a pre-immune bleed (5 ml per rabbit) was performed to collect Control Serum. On Day 1, each rabbit was immunized with 0.50 mg of antigen in CFA at 10 s.q. sites to provide the primary injection. Booster immunizations were carried out on days 14, 28 and 42. In particular, on Day 14, each rabbit was boosted with 0.25 mg of antigen in IFA at 4 s.q. sites. On Day 28, each rabbit was again boosted with 0.25 mg of antigen in IFA at 4 s.q. sites. Serum samples were collected from each rabbit after 35, 56, and 58 days post-immunization. In particular, on Day 35 each rabbit was bled to obtain about 25 ml. A third booster was administered to each rabbit on Day 42, comprising 0.25 mg of antigen in IFA at 4 s.q. sites. On Days 56 and 58, each rabbit was again bled twice to obtain about 50 ml.

The antibodies against the CDON N-terminal peptide were purified from serum using AminoLink Immobilization kit (Thermo Fisher Scientific #44890) as per the manufacturer's instructions. The antibodies against the CDON C-terminal peptide were purified from serum using SulfoLink Immobilization kit (Thermo Fisher Scientific #44999) as per manufacturer's instructions.

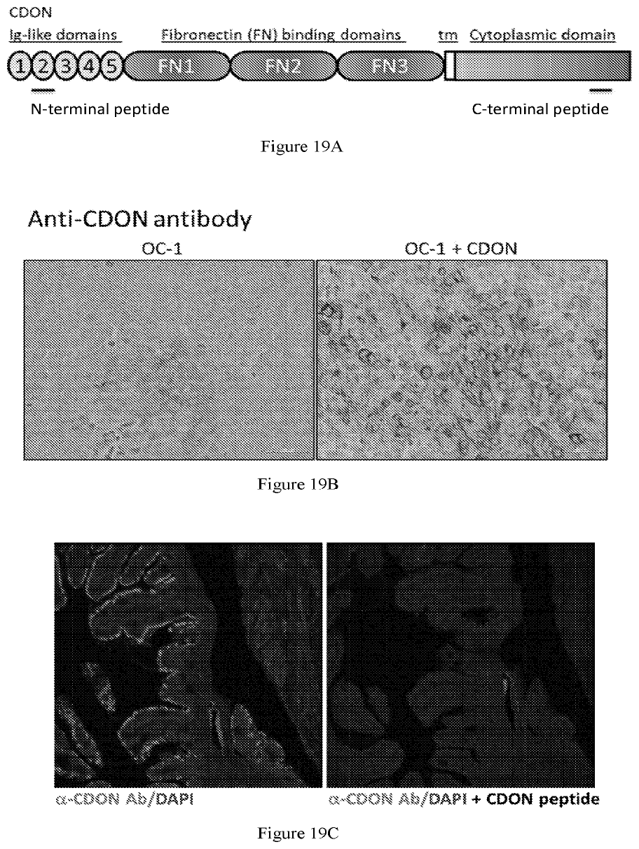

The antibodies were examined for recognition of CDON via immunoblot (FIG. 18). Briefly, protein lysates expressing endogenous basal CDON and containing tagged-CDON overexpression were separated via SDS-Page and probed with each antibody. The antibodies were further examined for reactivity in immunohistochemistry using FFPE tumors comprised of ovarian cancer cells expressing a control plasmid or tagged-CDON overexpression as well as whole murine reproductive tracts. Additional immunohistochemical testing was carried out on tissues collected from xenografts of patient-derived ovarian carcinoma cells (OC-1) expressing endogenous levels of CDON and OC-1 cells transduced with a CDON expression construct. This analysis confirmed detection of a band of correct size (130 kDa) by western blot and increased cytoplasmic and membranous signal in CDON over-expressing xenograft tissue compared with controls with sera produced from the C-terminal peptide (FIG. 19B and data not shown). Tissue and cultured cells were evaluated by immunofluorescence (IF) staining, again showing good detection of CDON using this method. Further, the specificity of the antibody was shown by competition of the signal in the presence of exogenous peptide (FIG. 19C). These results demonstrate successful isolation of anti-CDON antibodies useful for detection using several methods. The utility of these antibodies will be further analyzed in FACS experiments to reliably isolate CDON⁺ cells for analysis of stem-like cell markers and functional properties.

The antibodies were utilized in immunofluorescent staining of whole murine reproductive tracts and tumor samples and in in vitro testing to assay phenotypic response of cell lines and binding in culture. Briefly, 0-10 μg/ml of antibody was added to adherent or non-adherent cells at the time of plating or after 24-48 hours after cells were plated. Cells were incubated for 72-96 hours and observed/assayed for appearance or viability. For analysis of antibody binding to cells, FACS was used on adherent and non-adherent cells with secondary only, no antibody and commercially available antibody controls.

### Example 16: CSC Characteristics of CDON⁺ Cells

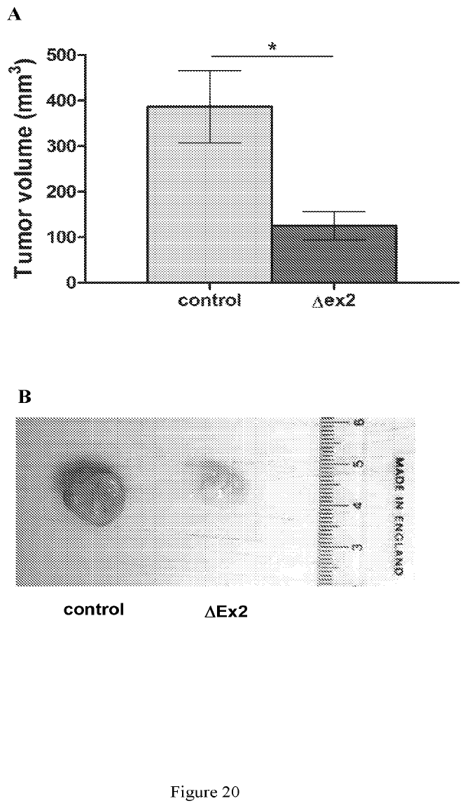

To examine the effects of CDON depletion on tumor engraftment, NSG mice were engrafted with patient-derived OC-1 cells transduced with a CRISPR/Cas9 non-targeting (control) gRNA or with a CDON targeting gRNA (ΔEx2) that results in depletion by targeting deletion at exon 2 of the CDON gene. Equal numbers of drug-selected transduced cells were injected into the left (control) and right (ΔEx2) flanks of female NSG mice (n=7 mice). Tumors were allowed to grow and mice were euthanized and tumor tissue was collected and measured with calipers to calculate tumor volume. Tumors with CRISPR/Cas9-mediate CDON depletion were significantly smaller than the control group (FIG. 20).

### Example 17: Comparative Drug Sensitivity or Resistance of CDON⁺ Putative CSCs

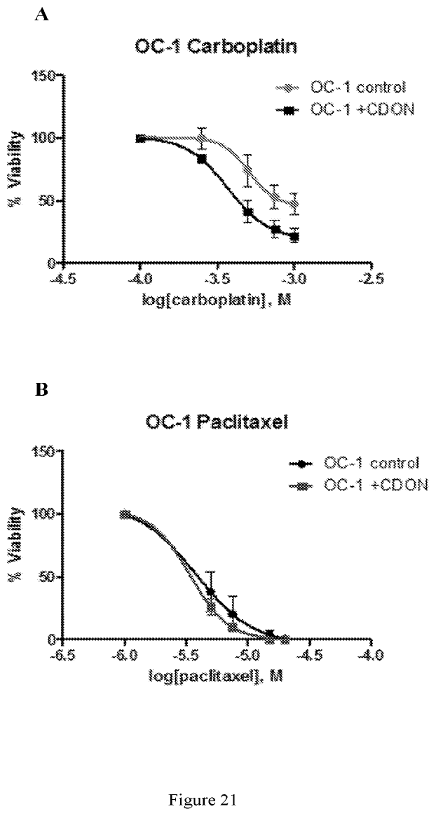

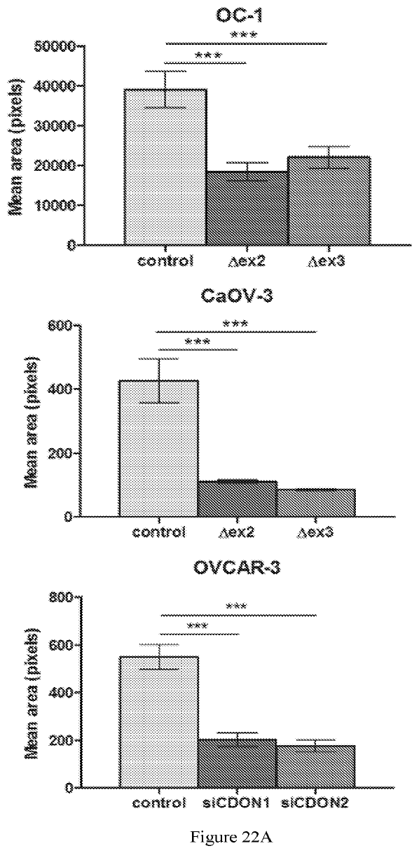

Experiments were conducted to optimize numbers of cells for plating and determine drug sensitivity (inhibitory concentration, IC50) for individual cell lines to standard chemotherapeutic agents carboplatin and paclitaxel (data not shown). After IC50 values were established, the sensitivity of cells with altered CDON expression was assessed (FIG. 21 and data not shown). Experiments showed that expression of a CDON cDNA construct in patient-derived ovarian carcinoma cells (OC-1) sensitizes cells to treatment with carboplatin and show little change in sensitivity to paclitaxel. Conversely, CRISPR/Cas9-mediated depletion CDON resulted in decreased in sensitivity to carboplatin.

### Example 18: Determining the Requirement and Mechanism of CDON Function for OC Multicellular Tumor Spheroid Formation

### Determining the Effects of CDON Depletion on Multicellular Spheroid Formation.

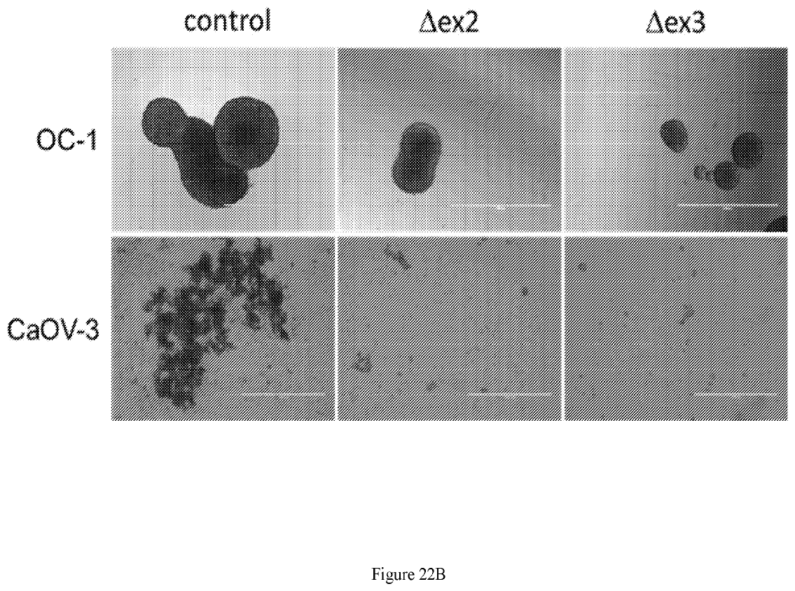

The analysis of sphere forming capacity has been extended by repeating independent experiments in OC-1 cells (FIGS. 22A and 22B) and CRISPR/Cas9-mediated deletion and/or siRNA depletion of CDON in two additional OC cell lines for a total analysis of three independent cell lines. Notably, following transduction of the CRISPR/Cas-9 CDON deletion constructs (using independent gRNAs designed to mediate deletion at exon 2 or exon 3), was insufficient to isolate stable clones of cells with complete deletion of CDON. This is consistent with the early experience with OC-1 cells and further underscores the importance and/or requirement of CDON expression for cell viability/survival. To ensure robust data, for each cell line and assay performed, all experiments were performed with a minimum of three technical replicates and three independent experiments prior to final data analysis.

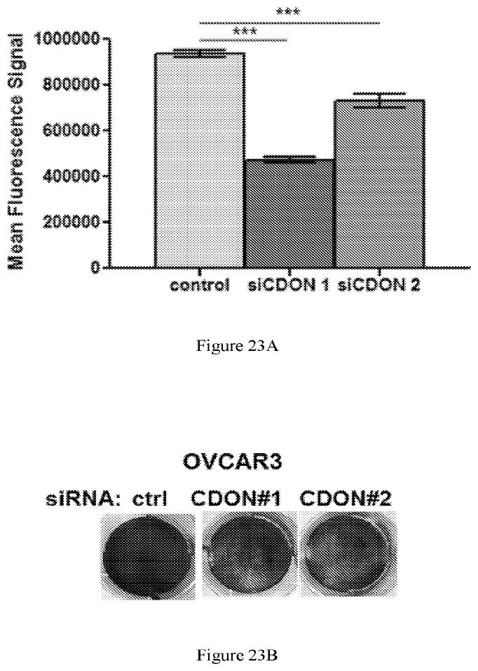

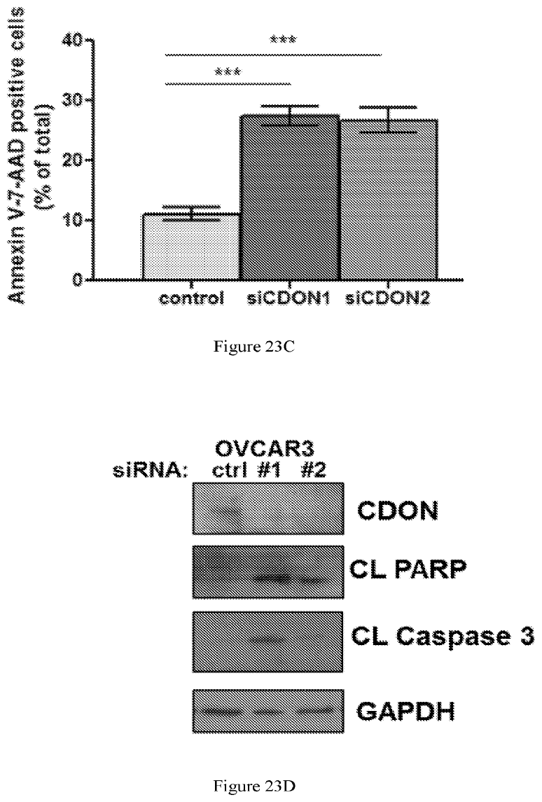

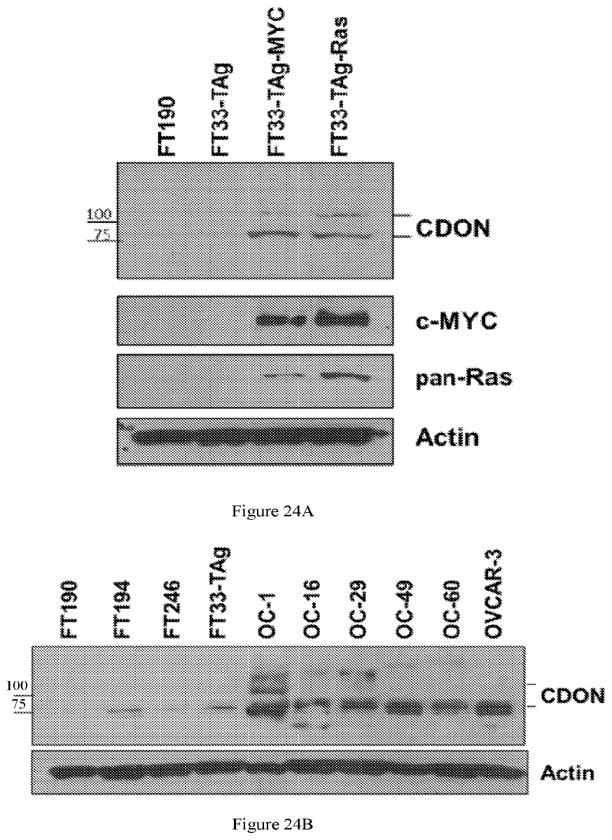

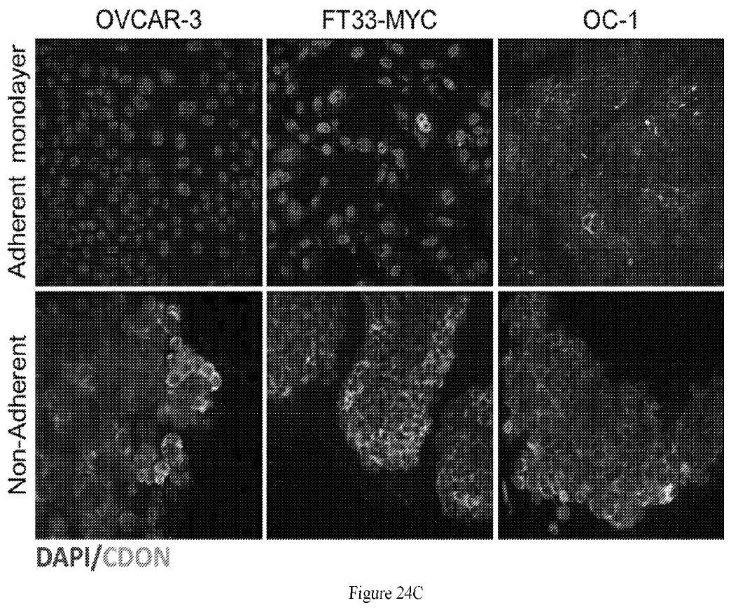

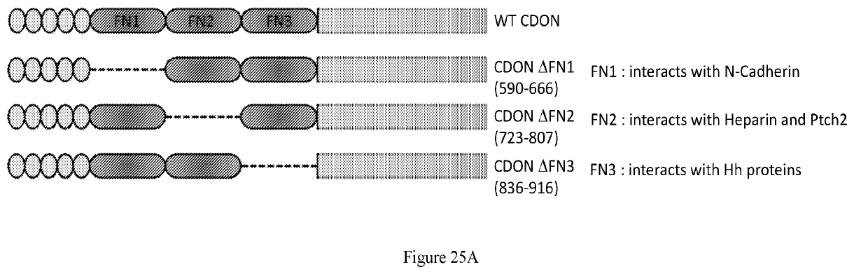

Tumor sphere formation was analyzed as described previously and showed significant reduction of tumor sphere size and tumor sphere forming efficiency in all three cell lines (FIGS. 22A and 22B, and data not shown). Proliferation was analyzed by CyQUANT™ Cell Proliferation Assay (ThermoFisher) to assess DNA content, by colony formation and by analysis of mRNA levels of cell cycle inhibitors P21 and P27. Cell death was measured by Annexin V assay and analysis of cleaved PARP and cleaved Caspase-3 by western blot (FIGS. 22A and 22B, and data not shown). Results of these experiments show that depletion of CDON results in significantly decreased proliferation capacity and increased cell death (FIGS. 23A, 23B, 23C, and 23D and data not shown).

### The Effects of CDON Depletion on Expression of HH Pathway Genes.

To determine whether OC cell lines secrete Shh under standard culture conditions (growth in serum) or following serum starvation andre-stimulation with serum or cholesterol. Shh levels were measured using a human sonic hedgehog ELISA kit (Abeam) and compared to an established pancreatic adenocarcinoma cell line, BxPC3, that has been demonstrated to secrete robust levels of Shh under various conditions. This analysis showed that CaOV-3 cells secrete little or no measureable Shh by this assay under any of the conditions tested. In order to determine whether OC cell lines could respond to exogenous Hh stimulation therefore an expression construct was utilized to produce Shh in HEK-293TL cells, collected and filtered Shh-containing medium, and treated OC cells with this conditioned medium for 48 hours. Preliminary results showed Hh canonical target genes, including Gli1, and its downstream target SFRP1 were activated by exogenous Hh. Induction of these factors is dampened in cells containing enhanced CDON expression. Preliminary studies to assess changes in Hh pathway genes upon depletion of CDON in OC cells have produced consistent data showing that depletion of CDON protein increases Gli 1 mRNA expression. Notably, the D. melanogaster homolog of CDON, Boi, plays a critical role in controlling Hh signaling in the ovary by binding and sequestration Hh protein.

### The Effects of CDON Depletion on Adhesion Proteins.

The new anti-CDON antibodies were used to confirm that OC cells (OVCAR-3 and OC-1) and immortalized and transformed FTSEC cells (FT33-MYC) exhibit profound differences in CDON protein expression that is dependent on cell culture conditions (FIGS. 24A and 24B). When these cells are grown as 2D monolayer cultures on adherent cell culture dishes, they exhibit few to rare cells with detectable CDON protein expression (FIG. 24C). When the same cells are grown as 3D clusters by plating in low-adhesion culture dishes, there is a striking increase in the amount of CDON protein expression (FIG. 24C). This has been shown in OVCAR-3 and OC-1 cells previously with a commercial antibody. The clear staining using the new antibody demonstrates its utility for IF assays. The prominent increase of CDON protein in FT33-MYC cells grown as 3D clusters is shown here for the first time.

### Defining Structural Domains Necessary for CDON Functions in OC Cells.

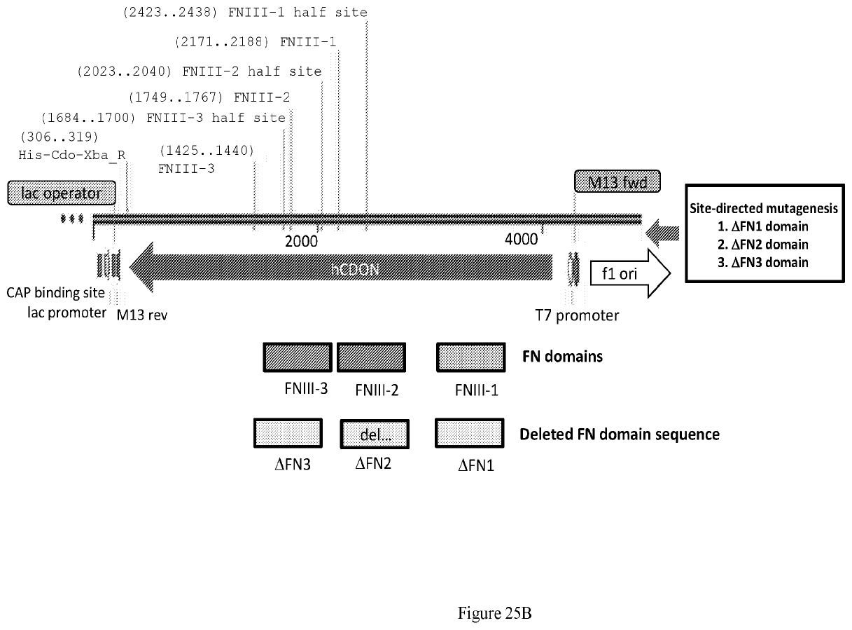

Mutant constructs of all three fibronectin Ill-like domains were generated (FIGS. 25 A and 25B, schematically depicted as FN1, FN2 and FN3). Each of these domains is involved in key protein-protein interactions that mediate CDON signaling: FN1 contains a binding site for N-cadherin; FN2 contains binding sites for heparin and PTH2 and FN3 contains a binding site for Hh proteins. Briefly, deletion of each of the three fibronectin III-like domains (ΔFN1, ΔFN2 and ΔFN3) was achieved by site-directed mutagenesis using Agilent QuikChange XL on WT full length CDON in the plvx lentiviral vector or in a smaller pcr2.1 cloning vector. Individual bacterial clones were isolated, subjected to restriction enzyme digestion analysis to detect deletions and then sequence verified to determine accurate deletion of intended sequences and absence of any PCR induced changes. The resulting sequence verified plasmids plvx.HIS-CDON ~FNI, pcr2.1 HIS-CDON ~FN2 and pcr2.1 HIS-CDON ~FN3 which each contain complete deletion of the respective fibronectin III-like domain.

### Example 19: CDON is a Marker of Cancer Stem Cells (CSC)

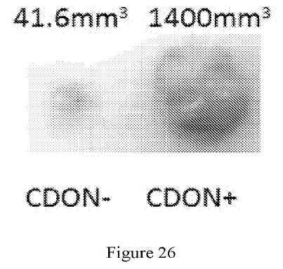

When equal numbers (1600) CDON⁺ and CDON-MIA-PaCa cells were injected into the flanks of Nod-SCID IL-2Ry^{-/-} (NSG) mice, CDON⁺ cells produced palpable tumors that reached a mean size of 1400 mm3 by 4 weeks while only one of the two injection sites of CDON-cells generated a tumor, measuring only 42 mm³ (FIG. 26). These tumors were heterogeneous, maintaining the same proportion of CDON⁺ cells as in the original cell line (0.2%), and could be serially propagated, providing strong preliminary evidence to suggest that CDON⁺ cells can self-renew and generate differentiated tumor.

### Example 20: CDON Expression in OC

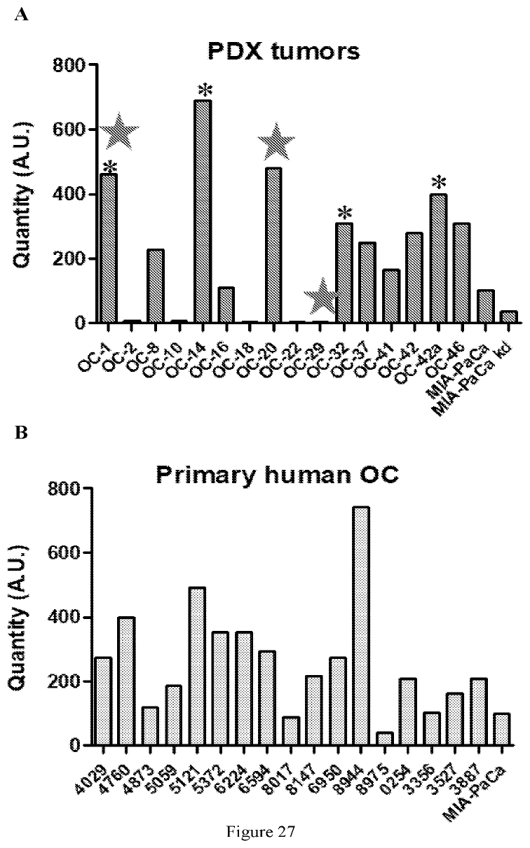

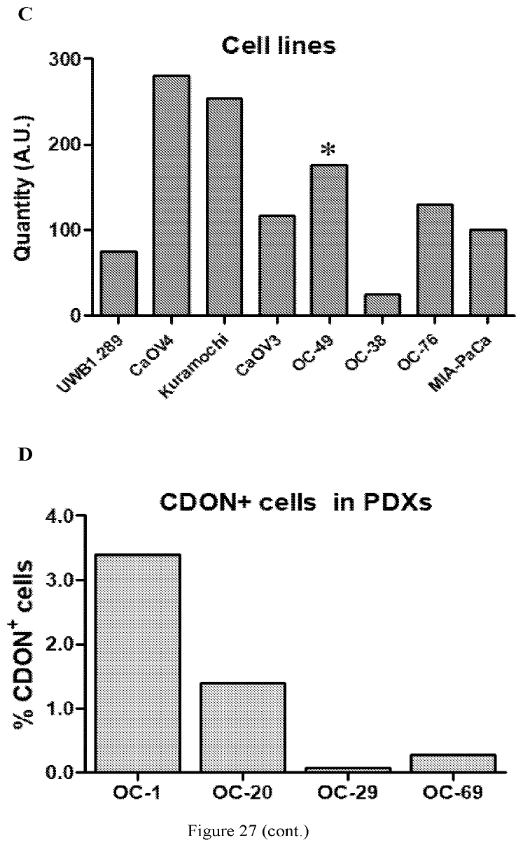

CDON mRNA expression was evaluated by RT-qPCR in OC-PDX tumors, primary OC specimens, and established and patient-derived OC cell lines. Using mRNA isolated from MIA-PaCa cells as a positive control, CDON expression was evaluated by real time quantitative polymerase chain reaction (RT-qPCR) of RNA isolated from novel OC PDX models a set of unrelated snap frozen primary OC tumor specimens and established OC cell lines. This analysis shows high levels of CDON expression in most cases. mRNA expression was exceptionally elevated in PDX cases derived from tumor cells present in patient ascites specimens (FIG. 27, OC-1, OC-14, OC-32, OC-42a and OC-49 labeled with asterisks). While mRNA levels are high, protein expression patterns are more complex. Similar to observations in mouse PDAC tumors and cell lines, CDON protein expression was detected only in a small percentage of cells isolated from primary PDX tumors or their matched cell lines grown in monolayer, varying from 0.02% to 3% (FIG. 27, Panel D).

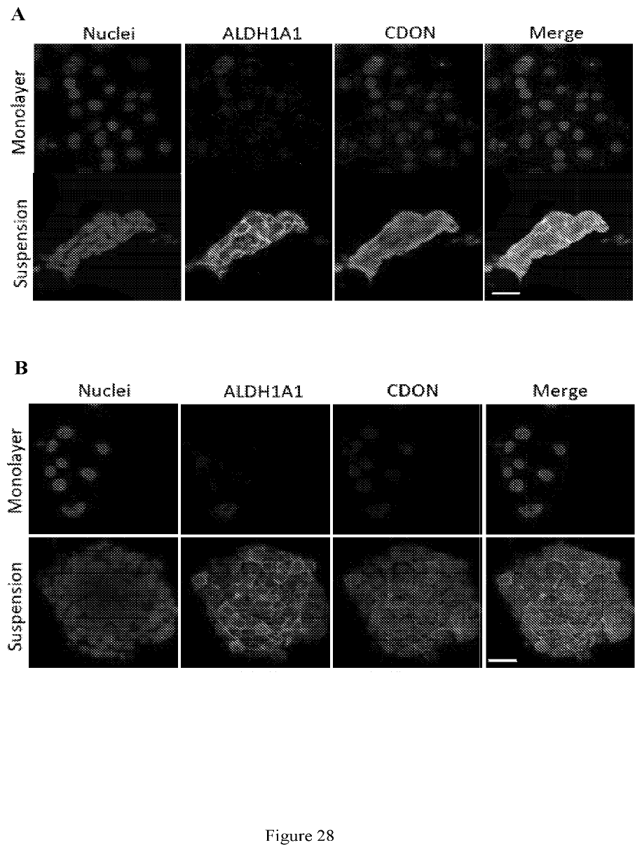

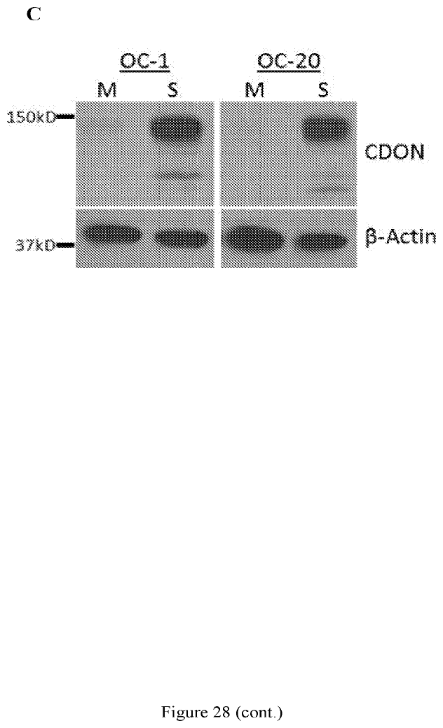

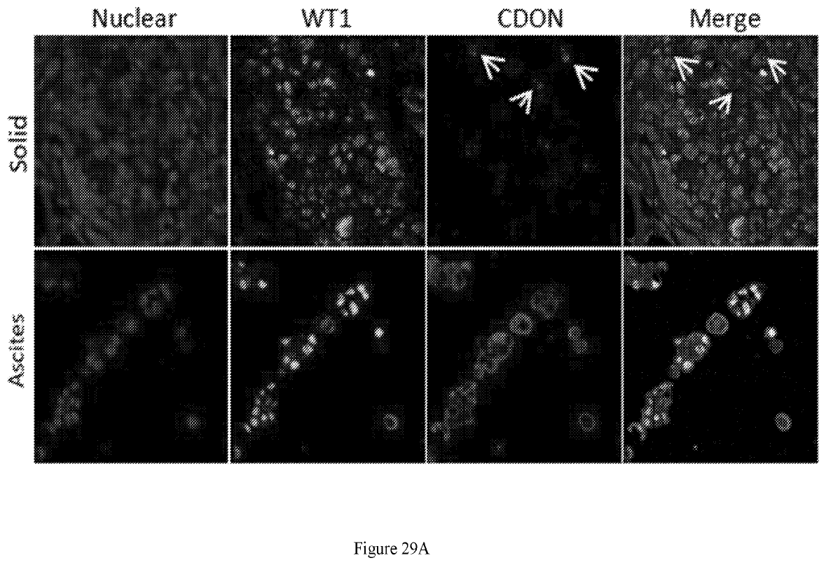

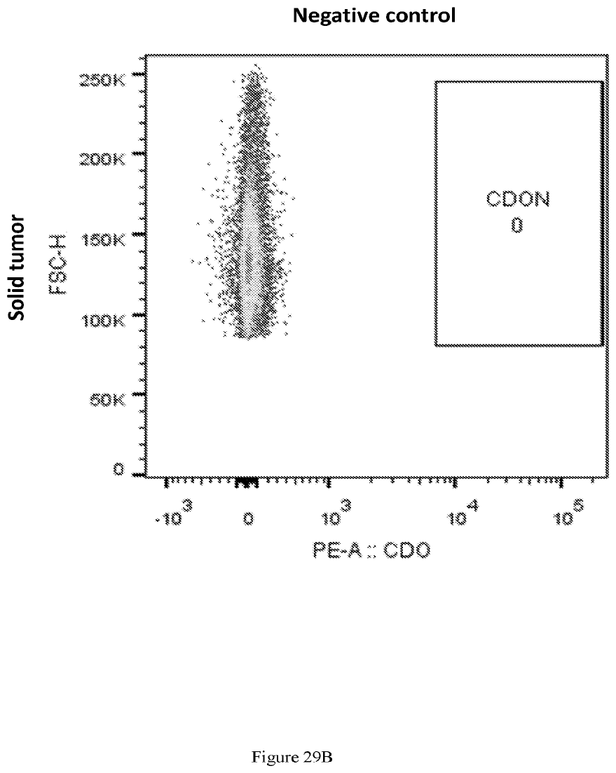

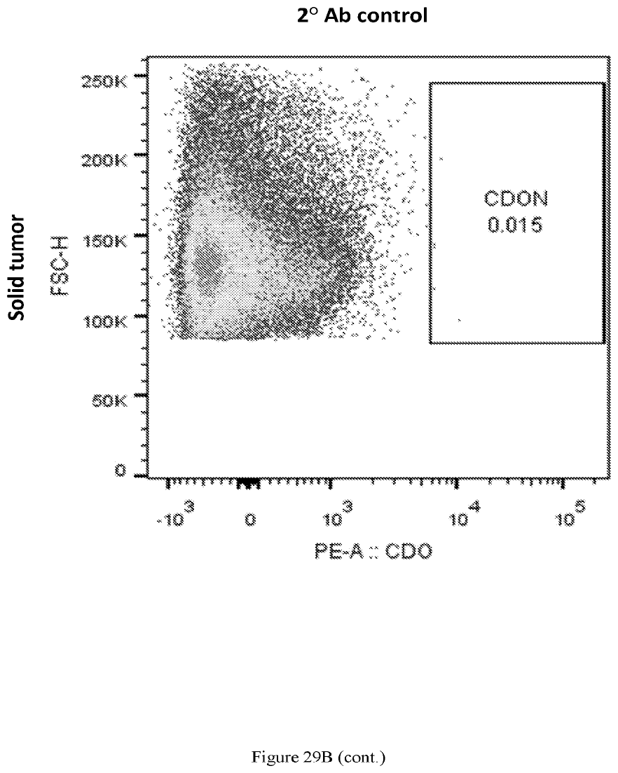

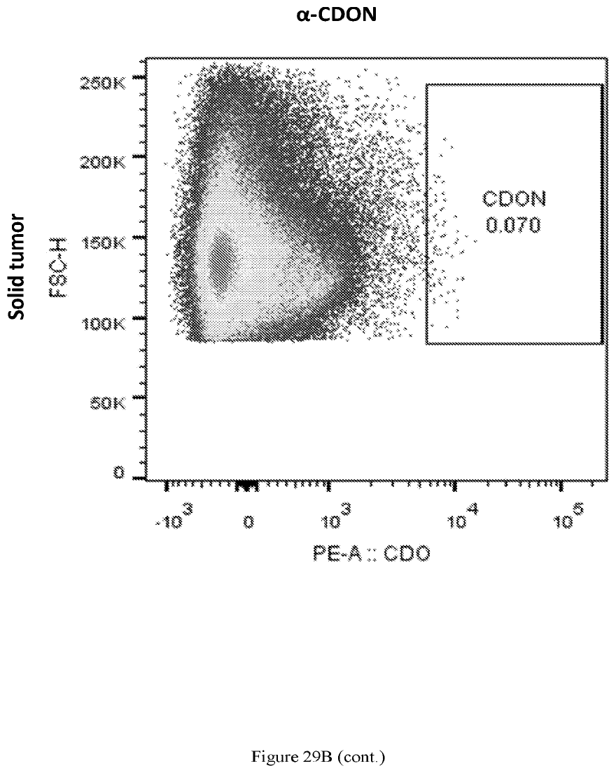

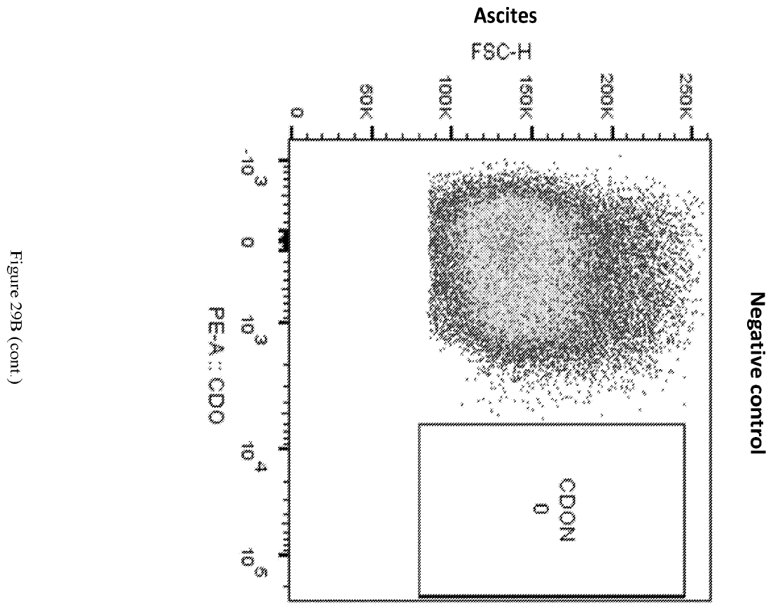

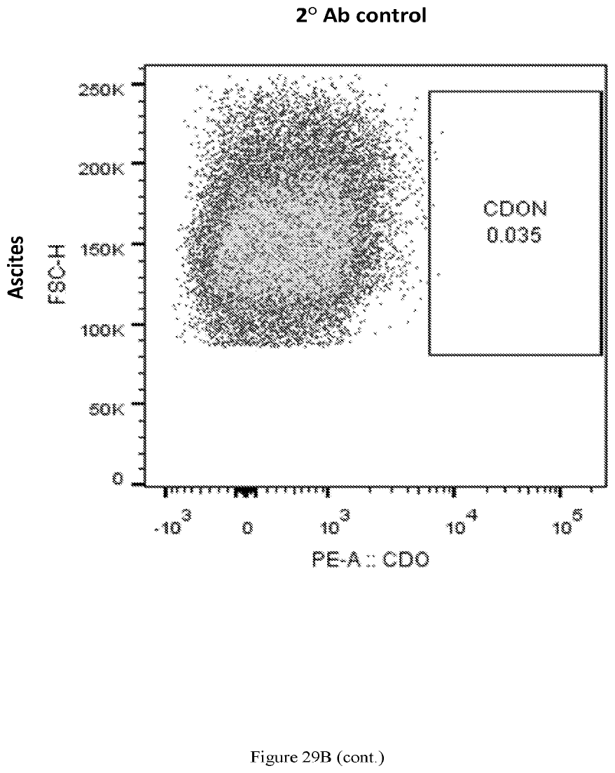

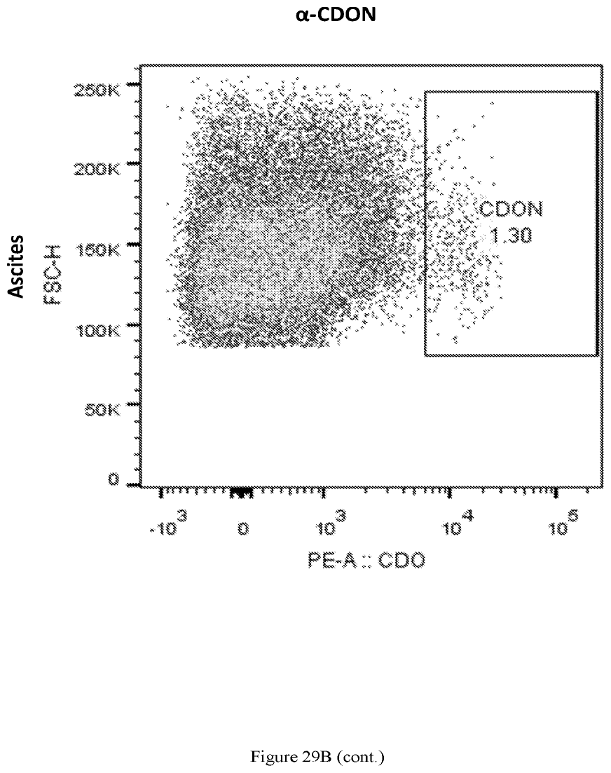

In monolayer cultures of high grade serous OC cell lines (Kuramochi, CaOV3, OC-1 and OC-20), cells exhibit little or no ALDH1A1 staining and only occasional CDON⁺ cells (FIG. 28, Panels A and B, and data not shown). Cells grown in suspension as spheroids exhibit enhanced ALDH1A1 expression (FIG. 28, Panels A and B). Unlike normal cultured rodent fibroblasts, where CDON expression is highest when cells cultured in 2D reach maximal confluence and are quiescent, OC cells grown in suspension exhibited strong CDON staining and total protein levels compared to the same cells grown in monolayer (FIG. 28, Panels A-C). Strikingly, CDON protein expression was largely overlapping with ALDH1A1. Similarly, these novel patient-derived OC cell lines cultured as 3D spheroids or organoids in Matrigel® also exhibited striking induction of ALDH1A1 and CDON protein expression (data not shown). These observations strongly suggest an important role for CDON in cell-cell adhesion in non-adherent growth. CDON protein expression patterns observed in OC PDX tumor models lend further support for this idea, where immunohistochemical (IHC) staining showed low CDON expression with only occasional CDON⁺ cells in solid tumors, but prominent expression in ascites (FIG. 29A). This increase in CDON protein in ascites was independently confirmed in fresh solid tumor and ascites analyzed by flow cytometry showing >18-fold increase in ascites (FIG. 29B).

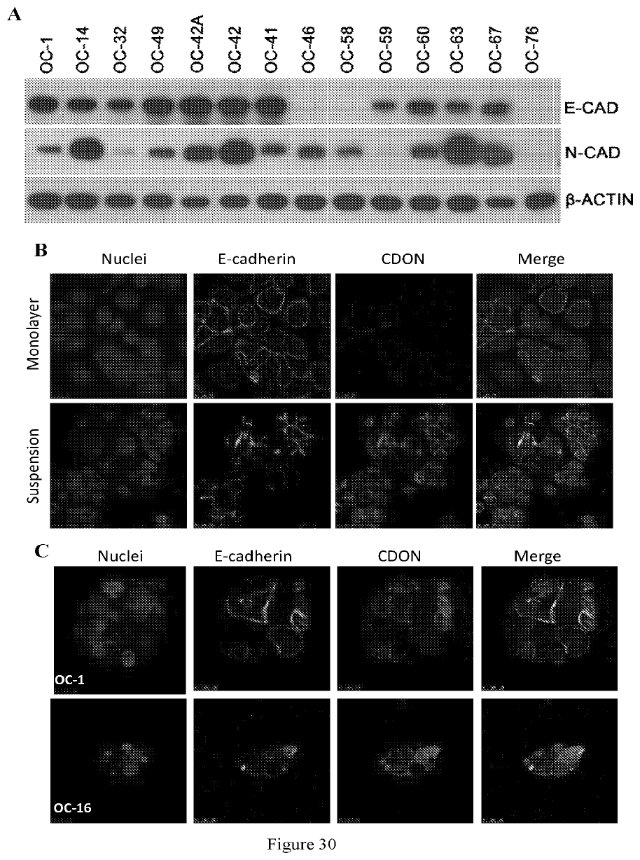

The functional role of CDON has been studied extensively in myogenesis and in this context CDON signals in a ligand-independent manner, with critical dependence on interactions with cadherins and downstream signaling mediated by β-catenin, CDC42 and MYOD. This mechanism is particularly intriguing because unlike many other solid tumors, published reports suggest that OCs frequently express both E- and N-cadherin (FIG. 30, Panel A). To further investigate this link, the expression and colocalization of CDON and E-cadherin in cells grown under adherent and non-adherent conditions was evaluated. This analysis showed expression of CDON is also closely aligned with E-cadherin expression in OC cells grown under nonadherent conditions (FIG. 30, Panels B and C).

### Example 21: Novel Patient-Derived Xenograft (PDX) Models of OC

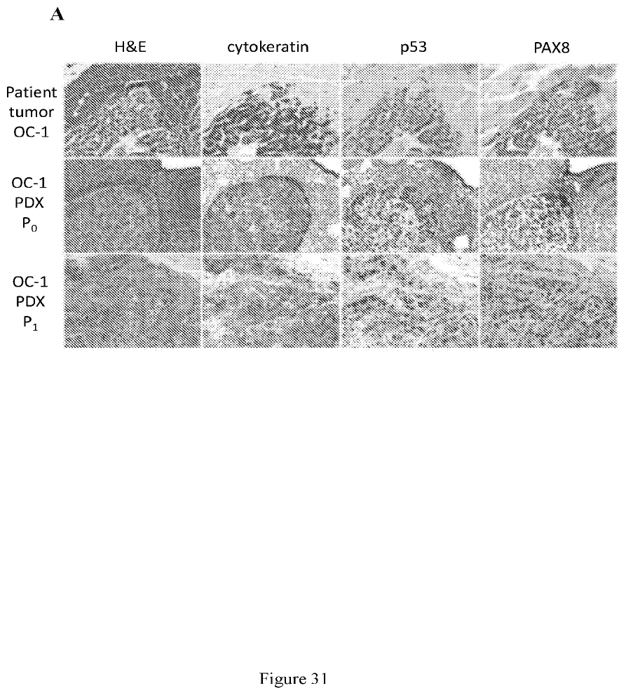

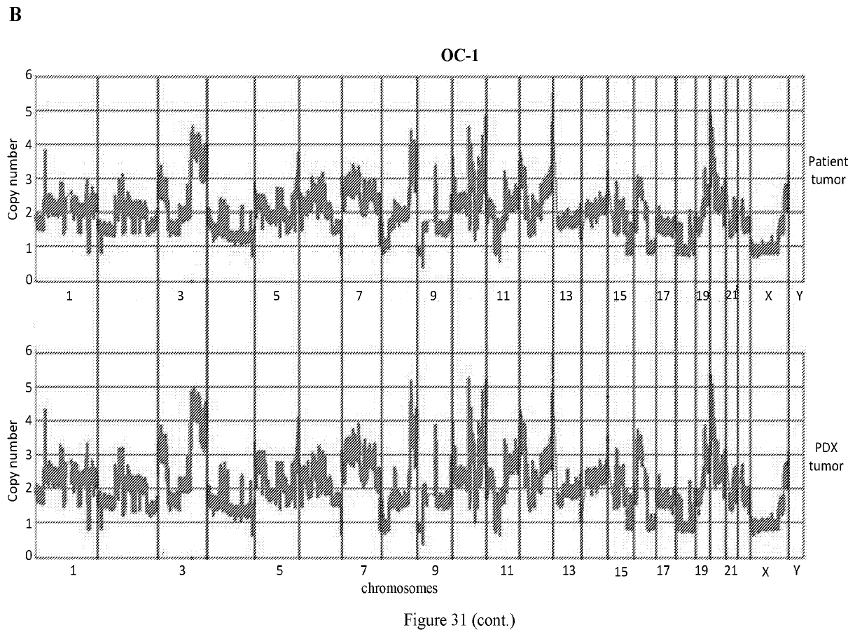

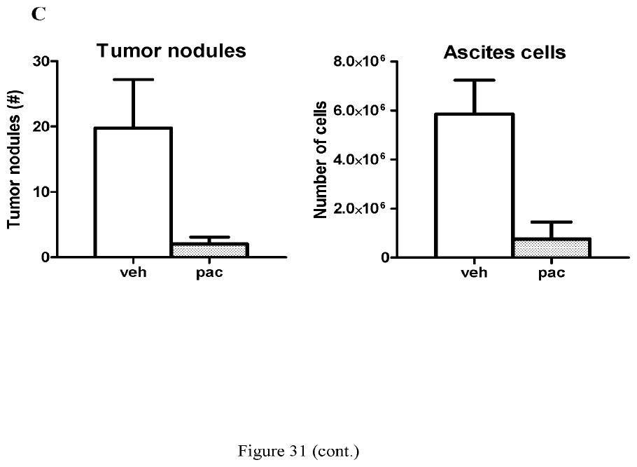

Direct implantation of fresh patient tumor tissue in mice results in biologically stable tumors with similar morphology, histopathological features, molecular alterations and inter- and intra-tumoral heterogeneity of patient tumors. A panel of >35 novel OC PDX models using fresh deidentified tumor tissue and ascites were propagated directly in mice. In parallel, matching cell lines for several PDX models by growing disaggregated tumor cells on irradiated fibroblasts in the presence of a Rho kinase inhibitor were established. These OC PDX models have been rigorously evaluated and maintain histopathological features, consistent marker expression and molecular features between the original patient tumor, the first graft (Pₒ) and subsequent passages (P_{1,2}, etc.) in mice (FIG. 31, Panels A and B, and data not shown). Deidentified clinical data is available for all PDX models. Drug treatment data associated with patient tumor OC-1 predicted its sensitivity to paclitaxel; this was validated in vivo, where paclitaxeltreated mice exhibited significantly fewer peritoneal tumor nodules and reduced number tumor cells in ascites compared to vehicle-treated controls (FIG. 31, Panel C).

In addition to copy number analysis (FIG. 31, Panel B) by array comprehensive genome hybridizaton (aCGH), high throughput RNA sequencing (RNA-Seq) analysis was performed on a number of patient tumors and corresponding OC-PDXs. These data were interrogated to determine relative expression of genes relevant to the proposed studies, including CDON and HH-pathway genes smoothened (SMO), sonic hedgehog (SHH) and GLI transcription factors (GLI1, GLI2, and GLI3). OC PDXs expressing CDON also express SMO and GLIs suggesting the capacity for HH signaling in these cells (data not shown). In addition, PDXs show common expression of E- and N-cadherin (CDH1,CDH2), cyclin D1 (CCND1) and vimentin (VIM), with low expression of snail 1 and 2 (SNAI1, SNAI2) and zinc finger e-box binding homeobox 2 (ZEB2), genes involved in epithelial to mesenchymal transition (EMT).

Results in PDAC and published studies demonstrating a role for SHH in OC CSC self-renewal and proliferation prompted the evaluation of CDON, ALDH1A1 and E-cadherin expression under 2D and 3D growth conditions in OC-PDX tumors and cell lines, with results showing consistent and prominent induction of CDON protein expression when cells are cultured in 3D on ultra-low attachment plates or as organoids in semi-solid media (e.g., matrigel, soft agar or reduced growth factor basement membrane, or as ascites in mice. A subset of 9 PDX models was prioritized (OC-1, -14, -16, -20, -29, -38, -42, -49 and -60) to be used for experiments proposed in this project based on: 1) their classification as high grade serous carcinomas (HGSC); 2) the availability of molecular data (aCGH and RNASeq); 3) availability of matched cell lines for 6/9 cases; 4) models that were derived from solid tumor (n=6) and ascites (n=3); and 5) in vivo growth properties.

### Example 22: CDON as a Functional CSC Marker

While markers of OC CSCs have been identified, there is little evidence for a functional connection of these markers to CSC phenotype. Critical properties of CSCs include self-renewal and the capacity to generate differentiated daughter cells that comprise a tumor. The observation that CDON⁺ cells serially generate heterogeneous pancreatic tumors in NSG mice is a strong indication that these cells have CSC properties. The low level and frequency of expression of CDON protein in OC cells and solid tumors also suggest that it may be a marker of OC CSCs. Finally, the ability of a small number of CDON⁺ ovarian cancer cells to produce spheroids supports the idea that CDON is a CSC marker in OC. An alternative, perhaps not mutually exclusive idea is that based on its known functions in myoblast differentiation and HH signaling, CDON may functionally contribute to the CSC phenotype.

Levels of CDON Expression and Co-Expression with Known CSC Markers in OC PDX Models

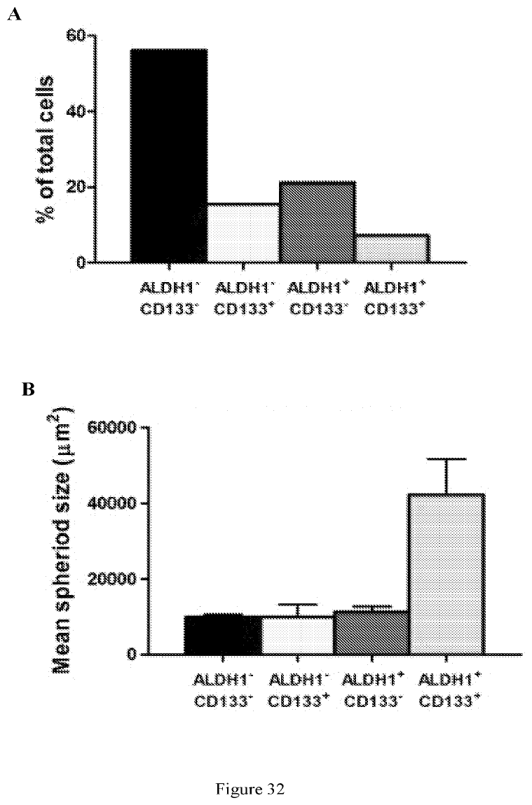

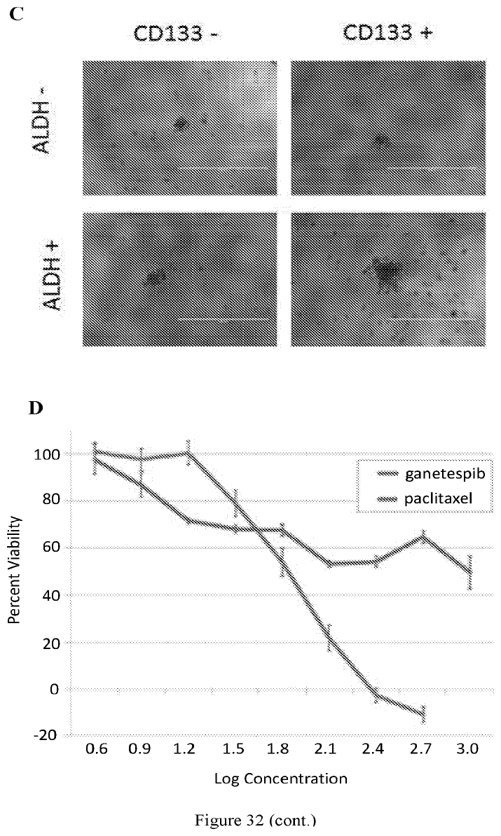

Using freshly isolated PDX tumor specimens grown in mice, CDON expression by flow cytometry and IF detection will analyzed. Selection for ALDH1′ and CD133′ can be utilized to enrich for OC cells with CSC properties including increased spheroid forming capacity and resistance to standard cytotoxic agents such a paclitaxel (FIG. 32). CDON⁺ cells may be a subset of these enriched populations; therefore, in addition to determination of the percent of CDON⁺ cells, co-expression of these markers with CDON will be tested. Preliminary data shows high levels of expression of CDON protein in OC cells grown under non-adherent conditions compared to cells grown in monolayer and in ascites compared to solid tumors. This suggests that ascites-derived models may have comparatively higher levels of CDON. With this in mind, CDON expression will be analyzed and compared in both solid tumor- and ascites-derived PDX models to stratify models with high (CDON^{hi}). intermediate (CDON^{int}) and low/negative (CDON^{low/neg}) expression. PDX cases derived from solid tumors include OC-16, -20, -29, -38 and -60 and cases derived from malignant cells present in ascites collected from paracentesis specimens include OC-1, -14, and -49. In addition, PDX model OC-42 was derived from both solid tumor (OC-42) and ascites (OC-42a) present at the time of primary surgery (a matched tumor/ascites model).

### Tumor Engraftment

Fresh tumor specimens will be generated by: 1) subcutaneous (s.c.) implantation of viable frozen tumor tissue fragments (1-2 mm³) to engraft solid tumor models OC-20, OC-29, and OC-42, and 2) intraperitoneal (i.p.) injection of 1×10⁷ cryopreserved ascites cells from models OC-1, OC-14, OC-42a OC-49. Two NSG mice (tissue donor mice) will be injected/model to generate sufficient exponentially growing fresh solid tumor or ascites for subsequent engraftment. Mice will be checked daily for wellness and to monitor tumor growth. Mice harboring s.c. tumors will be euthanized at or near the time tumors reach 500 mm³. Mice harboring ascites will be euthanized at or near the time they begin to exhibit mild abdominal distention (evidence of the presence of ascites) and tumor cells will be collected for subsequent injection. For analysis of CDON expression, NSG mice will be engrafted (n=2 mice/PDX model) by bilateral subcutaneous s.c. implantation of freshly tissue fragments (1-2 mm³) isolated from solid tumor models to generate 4 tumors (2 tumors/mouse, 2 mice/model) from each model. For ascites models, ascites from donor mice will be collected, red blood cells will be lysed, washed, disaggregated and cells will be enumerated (trypan blue exclusion and cell counting) for engraftment in mice (n=4 mice/model) by i.p. injection of 5×10⁶ cells. Engrafted mice will be checked daily for wellness and to monitor tumor/ascites development and collection using the same criteria as above. In mice with ascites, both floating ascites cells/multicellular aggregates and solid tumor nodules (if present) will be collected for subsequent analysis of CDON⁺, ALDH1⁺ and CD133⁺ expression.

### Analysis

Freshly collected tissue will be gently disaggregated to a single cell suspension using a gentle MACS tissue dissociator (Miltenyi Biotec) in preparation for marker analysis. Dissociated cells will be stained with anti-mouse H2K antibodies to exclude mouse cells from the analysis. To determine co-expression of CDON with ALDH1⁺ and CD133⁺ in primary solid tumors, tumor nodules and ascites, dissociated cells will be washed and labeled with anti-CDON and anti-CD133 antibodies. To detect ALDH enzymatic activity, cells will be subjected to the ALDEFLUOR Kit (Stem Cell Technologies) as described, with a portion of the cell/substrate preparation (20%) treated with diethylaminobenzaldehyde (DEAB) cells. Cell preparations will be stained with propidium iodide (PI) and subjected to flow cytometry analysis using PI staining to gate dead cells and ALDEFLUOR⁺DEAB to define negative gates. The flow cytometry analysis will be used for determination and quantification of cells expressing each individual marker and the fraction of CDON⁺ cells that express CDON⁺, ALDH1⁺ and/or CD133+. As an independent method, cytospin preparations of dissociated cells will be analyzed by IF imaging following staining with antibodies recognizing CDON, ALDH1A1 and CD133. Measurement of the location and number of CDON⁺, ALDH1A1⁺ and CD133⁺ cells will be performed using confocal microscopy and IMARIS evaluation software. Co-expression of CDON and ALDH1A1 occurs in OC cell lines and PDXs supporting the idea that CDON is a specific marker of CSCs.

### CSC Characteristics of CDON⁺ Cells

There are two gold standards for evaluation of CSCs. First, limiting dilution assays measure the ability of CSCs to: a) form spheroids comprised of CSCs and differentiated daughters, and b) form heterogeneous tumors in vivo. Second, isolated CSCs should be able to produce heterogeneous tumors sequentially upon serial transplantation. The CSC potential of CDON⁺ cells from PDX tumors (n=3 models) will be quantified by measuring in vitro spheroid formation and in vivo tumor formation upon limiting dilution and serial transplantation.

### Spheroid Formation

To assess spheroid forming capacity, single CDON⁺ and CDON⁻ cells isolated by flow cytometry will be cultured in low serum conditions (DMEM/F12 medium supplemented with 5 g/ml insulin, 20 ng/ml recombinant human epidermal growth factor (EGF), 10 ng/ml basic fibroblast growth factor (bFGF) and 0.4% fetal bovine serum) in ultra-low attachment plates. Two approaches will be used to evaluate sphere formation: 1) low density plating (5000 cells/ml) and 2) single cell (1 cell/well in 96 well plates). Spheroid formation will be monitored for 2 weeks and detection and enumeration of spheroids will be performed by capturing images of five random fields under bright field microscopy (Evos® Cell Imaging System) and analyzing images for the number and size of spheroids present using Image J. Assays will be performed in triplicate and the number and percent of spheres formed determined for CDON⁺, CDON⁻ and unsorted cells. The capacity for serial spheroid formation will be tested for two additional passages by disaggregating and sorting (CDON⁺, CDON⁻ and unsorted cells) cells from the spheres formed followed by low density plating as above. By cell sorting for serial passage analysis the percent of CDON⁺ and CDON⁻ cells in the spheres that formed will be determined. To evaluate CDON-mediated signaling proteins in spheres, aliquots of spheroids will be used for cytospin preparation and subsequent analysis of protein expression and activation (E- and Ncadherin, p38MAPK, AKT, FAK) by IF and confocal microscopy. OC spheroid formation has previously been shown to be significantly increased in the presence of HH (sonic or Indian hedgehog) suggesting the possibility that ligand-dependent CDON signaling mechanisms may also be important. Thus in separate assays, the effect of HH signaling on spheroid formation will be evaluated by plating CDON⁺, CDON⁻ and unsorted cells on ultra low attachment plates in the presence of recombinant SHH (250-800 ng/ml) or Hh agonists such as function blocking monoclonal antibody (El), cyclopamine or IPI·926 (saridegib).

### Tumor Formation in Mice

To determine whether CDON⁺ cells engraft more readily in mice and give rise to differentiated tumors (containing CDON⁺ and CDON-cells), tumor formation will be tested by limiting dilution. Fresh tumor tissue will be obtained by growth in Tumor donor' NSG mice (n=2-3 mice/model, based on expected numbers of CDON⁺ cells as described above to obtain fresh tumor tissue for subsequent FACS sorting and implantation of equal numbers of CDON⁺ and CDON⁻ cells in NSG mice. Mice will be euthanized and tumors collected, disaggregated to a single cell suspension and labeled with antimouse H2K (to gate out murine cells) and anti-CDON antibodies and subjected to FACS sorting. Sorted cells will be diluted and a total of 10,000, 5,000, 1000, 2500 and 500 CDON⁺ and CDON⁻ cells will be implanted by bilateral s.c. injection for solid tumors (n=3 mice/2 tumors mouse) and i.p. injection for ascites tumors (n=6 mice/model). The null hypothesis tested will be that the rate of tumor formation is the same for CDON⁺ and CDON⁻ cells versus the alternative that it is faster in CDON⁺ cells. The model tested will be single hit model associated with the extreme limited dilution assay (ELDA) that posits that a small fraction of injected cells will successfully engraft and that the chance of tumor formation at a single injection site is p=1−exp (⁻b n) where n in the number of injected cells and b×n is the average number of successful ones. Then p is the chance that at least one cell will succeed in causing a tumor to form assuming the Poisson distribution for this number. The number b⁻ for CDON⁻ cells is expected to be smaller than b⁺, that for CDON⁺ cells. The ratio b⁺/b⁻=1 under the null hypothesis. Distinguishable ratios depend on the underlying values of b⁻. With 6 injection sites/group, ratios >1.0 can be distinguished from 1.0 with at least 80% power and 5% type I error. Tumor incidence will be compared to determine if the frequency of tumor formation is higher in CDON⁺ cells and if significantly fewer CDON⁺ cells are required to initiate a tumor. Tumors that grow will be disaggregated, labeled with antimouse H2K and anti-CDON antibodies and subjected to FACS to evaluate the potential for tumors arising from CDON⁺ and CDON-cells give rise to differentiated tumors by determination of the percent of CDON⁺ and CDON⁻ cells present in the resulting tumors. The FACS sorted CDON⁺ and CDON⁻ cells from these tumors will be re'engrafted in NSG mice as described to establish whether they give rise to tumors upon serial transplantation in mice. This will be repeated in serially transplanted cells that engraft in mice.

### Example 23: Requirement for CDON for Tumor Formation

To determine the functional requirement of CDON for OC development and/or progression in vivo, a commercially available CRISPR (clustered regularly interspaced short palindromic repeats) Cas9 gene editing kit from Ori Gene (catalogue #KN214234) will be used for deletion of CDON in OC cell lines. This strategy is preferable as it avoids potential pitfalls such as off-target effects and/or selection for re-expression of the target often associated with RNA-interference via stably expressed shRNA constructs. This system consists of a donor vector containing the left and right homologous arms and a GFP-Puro functional cassette and two CDON-targeted pCAS-Guide RNA (gRNA) vectors. To facilitate analysis of in vivo tumor formation, OC cells (OC-1, OC-20, and CaOV3) will be transduced with a retroviral luciferase expression construct to enable in vivo bioluminescent imaging (BLI) to monitor tumor growth as described47-49. Luciferase expressing cell lines will then be transfected with the CRISPR/Cas9 donor and guides. Following transfection, time course experiments will be performed to determine when CDON mRNA and protein expression is lost using RTqPCR, flow cytometry, IF and western blot analyses as described above. Once loss of CDON gene and protein expression is confirmed, in vivo tumor growth will be compared following s.c. (n=4 mice/cell line) or i.p. (n=8 mice/cell line) implantation of equal numbers of isogenic cell lines with and without CDON. With 8 injection sites (bilateral flank or single i.p.) a difference of 60% in successful engraftment rates between CDON⁺ and CDON null cells can be distinguished from a rate difference of zero with 80% power and 5% type I error. Mice will be imaged weekly by BLI to monitor tumor in vivo tumor development. The incidence and extent of tumor formation will be compared to determine whether loss of CDON inhibits or abrogates tumor formation in mice.

### Example 24: Role of CDON in Drug Resistance

### Drug Treatment and Evaluation

First, equal numbers of parental cells (Kuramochi, CaOV3, OC-1 and OC-20 cells) with intact CDON will be plated in triplicate under adherent and non-adherent conditions, and allowed to grow for 24 hours prior to exposure to increasing concentrations of carboplatin (0-100 μM), paclitaxel (0-30 nM). Cells will be treated for 72 hours and the effects of drug treatment on cell viability will be evaluated by CellTiter-Glo luminescent cell viability assay and the drug concentration required to kill 50% of the cells (inhibitory concentration, IC₅₀) will be determined for each drug under each growth condition. Induction of apoptosis will be assayed by staining for Annexin V and propidium iodide. This analysis will be repeated for cells with CRISPR/Cas9-mediated deletion of CDON. Expression of CDON will be evaluated by IF and western blot in separately plated cells treated under the same conditions. Each experiment will be performed in triplicate and data analyzed using GraphPad Prism software to determine if observed differences in drug sensitivity are significant. Once ICs are established for each cell line, drug treatment and growth condition, this data will be used to design combination studies to determine the sensitivity of CDON+ and null cells to combined carboplatin and paclitaxel and the combination index (CI) using CompuSyn software as described.

OC CSCs may be sensitive to targeted small molecule therapeutics. For example, OC CSCs are reliant on JAK2/STAT3 pathway signaling and thus susceptible to small molecule JAK2 inhibitors. In addition, JAK2/STAT3 signaling in OC and the targeted blockade of JAK2/STAT3 pathway signaling with small molecule JAK2 inhibitors significantly reduce tumor growth and ascites production suggest the possibility that CDON expressing OC CSCs may be susceptible to JAK2 inhibition. To test this, the sensitivity of cells with intact CDON and isogenic cells with intact and CRISPR/Cas9 deleted CDON will be evaluated as described above. As JAK2 inhibitors exhibit low cytotoxicity in cells grown in 2D under adherent conditions, cells will be grown under both adherent and non-adherent conditions for 24 hours and treated with increasing concentrations of ruxolitinib (0-1 M) for 72 hours and cell viability, induction of apoptosis and analysis of CDON expression will be performed as described above. All experiments will be performed with three technical and three experimental replicates and data will be analyzed to determine the IC₅₀ of ruxolitinib for each condition.

Once the comparative drug sensitivities of CDON expressing cells is established, in vivo experiments will be performed to validate findings in cultured cells. To avoid the potential issues related to re-expression of CDON after FACS sorting, isogenic cells with intact and CRISPR/Cas9 deleted CDON will be used to test the relative sensitivity of cells to cytotoxic drugs (carboplatin and/or paclitaxel) and ruxolitinib. Mice will be injected with equal numbers of CDON⁺ or CDON⁻ tumor cells (n=5/group, bilateral flank tumors for solid tumors and n=10 mice/group, i.p. implantation for ascites models). If the fraction of resistant animals in the CDON⁺ group is at least 51% higher than that in the CDON-animals, drug resistance can be distinguished 80% power and 5% type I error based on a two-sample test of the binomial distribution. Once tumors reach 100 mm³ they will be randomized into treatment groups and treated using established drug doses, routes of administration and dosing schedules (e.g., 30 mg/kg carboplatin or 6 mg/kg paclitaxel by weekly intravenous injection for three weeks, 50 mg/kg ruxolitinib by daily gavage). Tumor growth will be monitored by BLI and quantified by caliper measurements and drug(s) effect will be determined (e.g., tumor growth, stasis or regression). A key prediction of the model is that cytotoxic drug treatment will kill bulk tumor cells but enrich for CSC populations. Conversely, use of an agent that targets CSCs such as ruxolitinib is predicted to reduce the proportion of CSCs and total tumor mass. Proportions of CDON⁺ cells in resulting tumors will be determined by FACS, if they are of sufficient size for this approach. In tumors that are too small for FACS sorting, CDON⁺ cells will be analyzed and quantified by IF of fixed tumor sections and using confocal microscopy and IMARIS software.

### Example 25: The Effects of CDON Depletion on Multicellular Spheroid Formation

The observation that CDON protein is markedly increased in established and patient-derived tumor OC cell lines grown on low attachment plates or as spheroids/organoids grown in semi-solid media suggests that CDON may play an important functional role in the capacity for OC cells to grow as multicellular aggregates in suspension, as is observed in ascites. Malignant cells present in ascites are thought to represent a particularly aggressive subpopulation of OC cells that exhibit increased CSC properties, including resistance to cytotoxic chemotherapy agents. The mechanism by which CDON contributes to multicellular aggregate formation is unclear, but could be related to its role as a receptor and mediator of HH signaling, to its ligand-independent functions as an adhesion receptor, or both.

CDON expression will be depleted by RNA interference (RNAi) or by gene editing using the CRISPR/Cas9 system. As a first approach for short-term assays, small interfering RNAs (siRNA) targeting CDON(CDON ON-TARGETplus SMART Pool siRNA, Dharmacon) will be transfected into cells with high CDON expression (Kuramochi, CaOV3, OC-1 and OC-20 cells). These cell lines are all derived from HGSCs; therefore, as a non-transformed control cell line, immortalized fallopian tube secretory epithelial cell (FTSEC) lines will be used. A fluorescent PPIB (cyclophilin B) targeting siRNA (siGLO cyclophilin, Dharmacon) has been used to optimize transfection conditions and will be used as a control construct for off target effects. Knockdown of CDON and PPIB will be confirmed by RT-qPCR and detection of protein levels by flow cytometry, immunofluorescence (IF) and/or western blot analysis. After confirming successful knockdown, the effects of CDON depletion on viability (CellTiter-Glo viability assay), apoptosis (Annexin V and propidium iodide) and multicellular sphere formation will be determined in cells grown in 2D and on ultralow attachment plates as described. In preliminary experiments (not shown) significantly increased expression of CDON was observed within 24 hours of plating under non-adherent conditions; thus, CDON is predicted to promote the association of tumor cells in suspension and depletion will result in significant inhibition or abrogation of multicellular spheroid formation. The number and size of multicellular spheroids that form in cells cultured on ultra-low attachment plates for 72 hours will be quantified as described above. Results observed in cells with siRNA-mediated depletion of CDON will be independently verified by the same methods in cell lines with CRISPR/Cas9 mediated deletion of CDON as they become available.

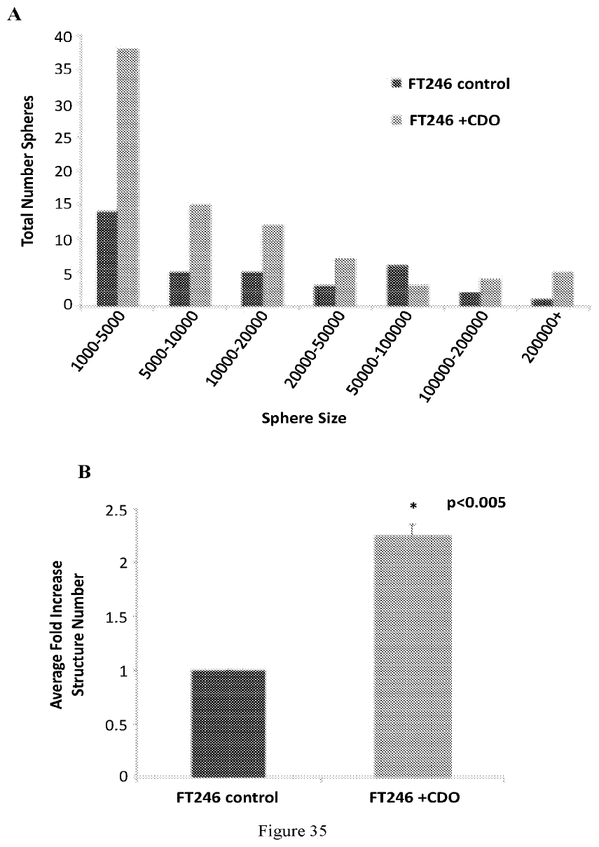

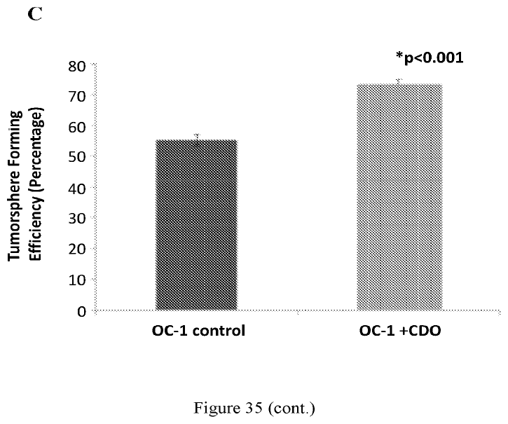

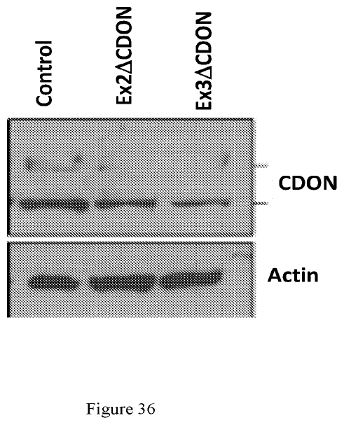

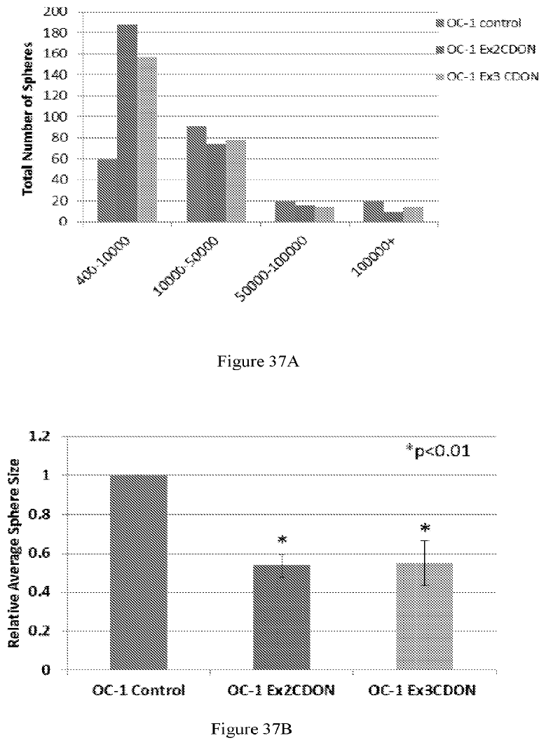

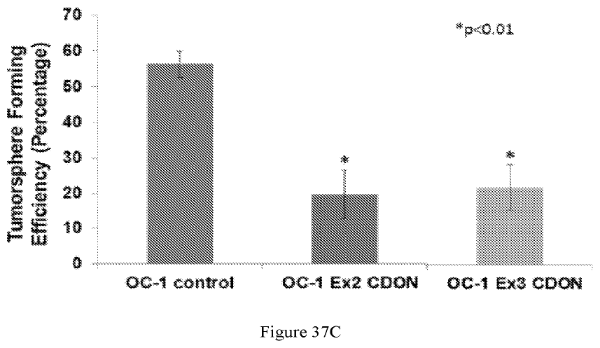

Preliminary experiments showed that CDON overexpression significantly enhances 3D spheroid formation (FIG. 35), a fundamental characteristic of cancer stem cells. Conversely, analysis of OC-1 cells with CRISPR/Cas9-mediated depletion of CDON demonstrated that cells with depleted CDON protein expression (FIG. 36) had diminished number and size of spheroids as well as sphere forming capacity from single cells (FIGS. 37A, 37B, and 37C). Taken together these results support a central role for CDON in spheroid formation in OC cells.

### Example 26: Effects of CDON Depletion on Expression of HH Pathway Genes

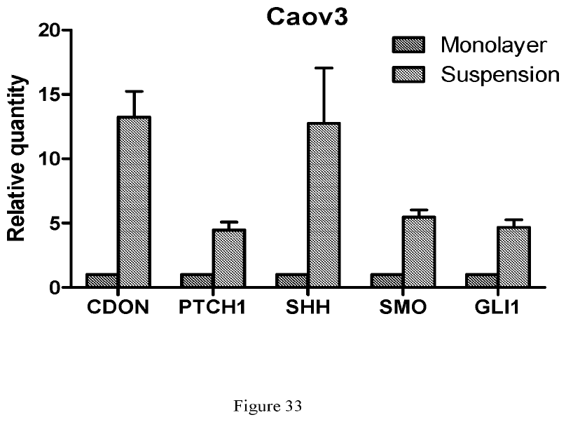

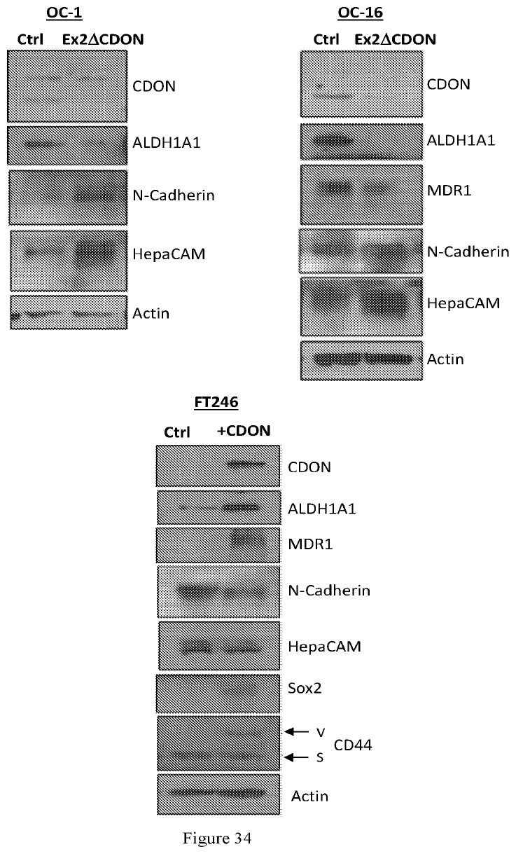

After observing the induction of CDON protein expression in OC grown in suspension, the expression of CDON and other HH pathway genes including PTCH1, SHH, SMO and GLI1 was evaluated by RT-qPCR in CaOV3, Kuramochi and UWB1.289 cells, and showed that expression of CDON and HH pathway genes is highly elevated in cells grown in suspension compared the same cells grown in monolayer (FIG. 33 and data not shown) suggesting that ligand-dependent functions of CDON via HH signaling may be important for multicellular spheroid formation. To investigate this further, the effects of siRNA-mediated CDON depletion and/or CRISPR/Cas9 mediated deletion on expression of HH pathway gene expression will be determined in cells grown in 2D monolayer and non-adherent conditions by RT-qPCR. Based on the prominent induction of SHH expression in cells cultured in suspension, the levels of secreted SHH by ELISA assay in cells with intact and depleted CDON will be also analyzed. As an alternative approach, cells with intact and depleted CDON will be treated with recombinant human SHH or with HH pathway antagonists (e.g., function blocking monoclonal antibody E1, cydopamine or IPI-926) to determine the effects of HH pathway manipulation on spheroid formation.

### Example 27: Effects of CDON Depletion on Adhesion Proteins

Studies of myoblast differentiation show that CDON is expressed with cell-cell adhesion proteins, particularly cadherins. Additional work also convincingly showed a link between extracellular matrix-via integrin engagement and FAK activation—to CDON expression and downstream signaling via Cdc42, p38MAPK, AKT and MyoD. E- and N-cadherins, integrins and activated FAK (pFAK^{Y397}) are key proteins involved in OC progression. The cell adhesion-mediated association of CDON expression with E- and N-cadherin and pFAK^{Y397} will be explored in OC cell lines (Kuramochi, CaOV3, OC-1, -16, -20). Preliminary experiments showed that CDON expression was closely aligned with E-cadherin expression in cells grown in 3D (FIG. 30) and that CDON protein expression was increased by 24 hours (data not shown). This will be further investigated systematically in time course experiments where expression of CDON will be established by IF analysis and confocal microscopy of cells at 1, 6, 24, 48 and 72 hours after plating on ultra-low attachment plates. Co-expression of CDON and E-Cadherin, N-Cadherin and pFAK^{Y397} will be evaluated by IF and western blot analysis in time course experiments. Downstream activation of p38MAPK, CDC42 and AKT will also be evaluated in cells with intact and CRISPR/Cas9 deleted CDON by Western blot analysis. It was recently shown that focal adhesion localization, activating phosphorylation and DNA binding activity of STAT3 is dependent on FAK activity in OC cells. Constitutive STAT3 activation is correlated with clinically aggressive behavior of tumors and poor patient survival and leads to increased expression of CCND1, BCL-XL and MCL-1 and VEGF. Independent work showed prominent STAT3 activation and upregulation of nanog, c-MYC and CCND1 in a subset of CD24+OC cells with aggressive behavior and CSC properties. Since CDON signaling in myoblasts is linked to activation of FAK, the expression and correlation of CDON will be compared with pFAK^{Y397}, STAT3^{Y705}, CCND1, BCL-XL and MCL-1 and VEGF, nanog and c-MYC in monolayer and suspension cultured cells. Associations will be subsequently be evaluated in cells with CRISPR/Cas9 deleted of CDON to validate the relationship to CDON.

Preliminary results showed CDON depleted cells (OC-1 and OC-16 cells), levels of ALDH1A1 and MDR1 were reduced, while HepaCAM (an adhesion molecule that negatively regulates growth) and N-cadherin levels were increased. Conversely, in cells with enforced expression of CDON, increased ALDH1A1, MDR1, CD44v isoform and SOX2 (associated with increased migration and invasion of ovarian carcinoma cells) and reduction of HepaCAM and N-Cadherin were observed (FIG. 34).

### Example 28: CDON Monoclonal Antibody Study

A second fusion and hybridoma production was carried out. The remainder of the cryopreserved splenocytes from mouse 5 (M5), and the splenocytes were fused with the fusion partner (SP20 cells), selected, and grown for 13 days.

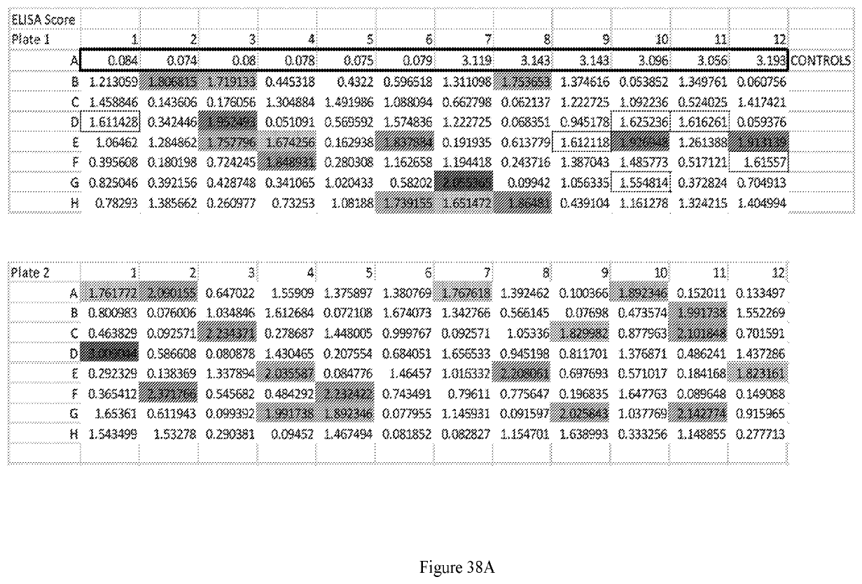

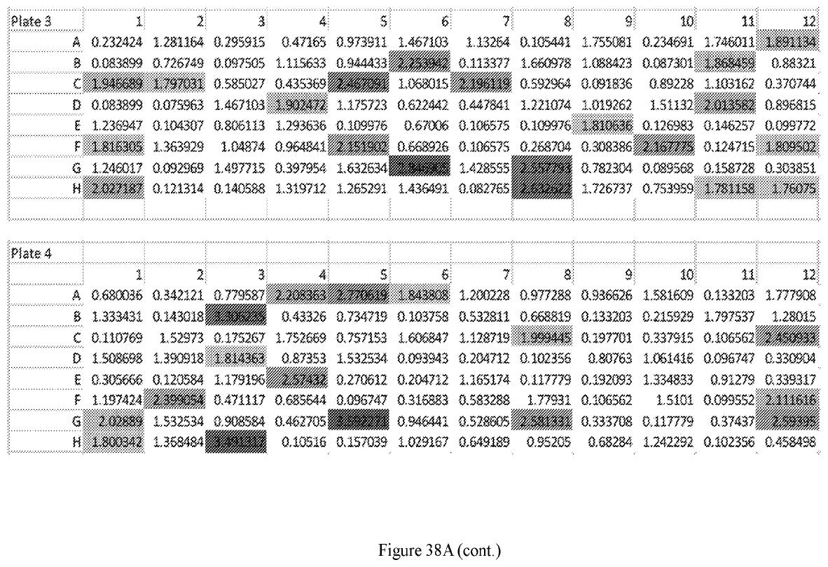

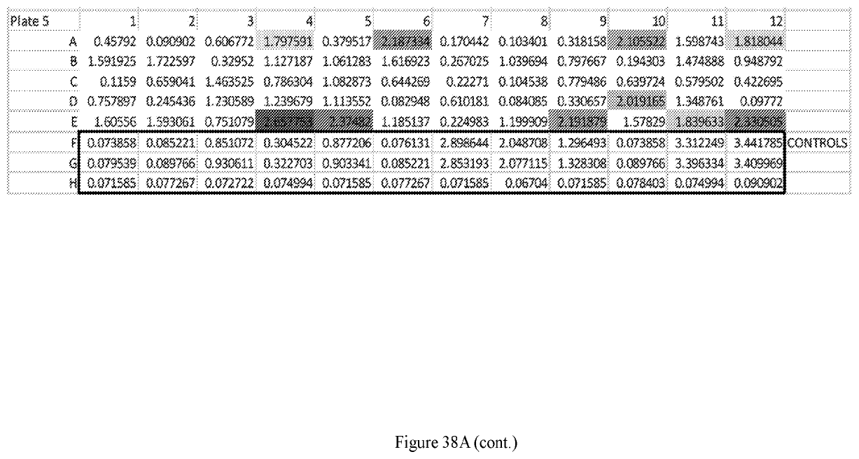

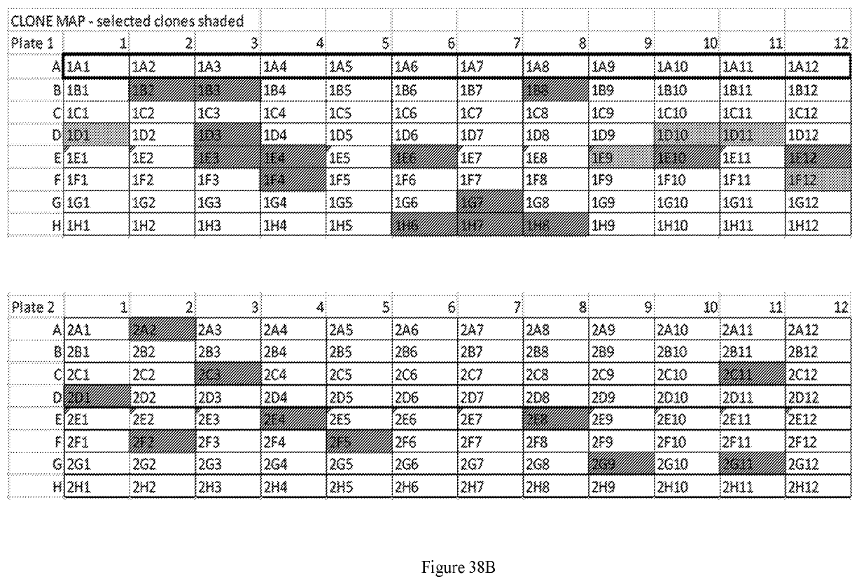

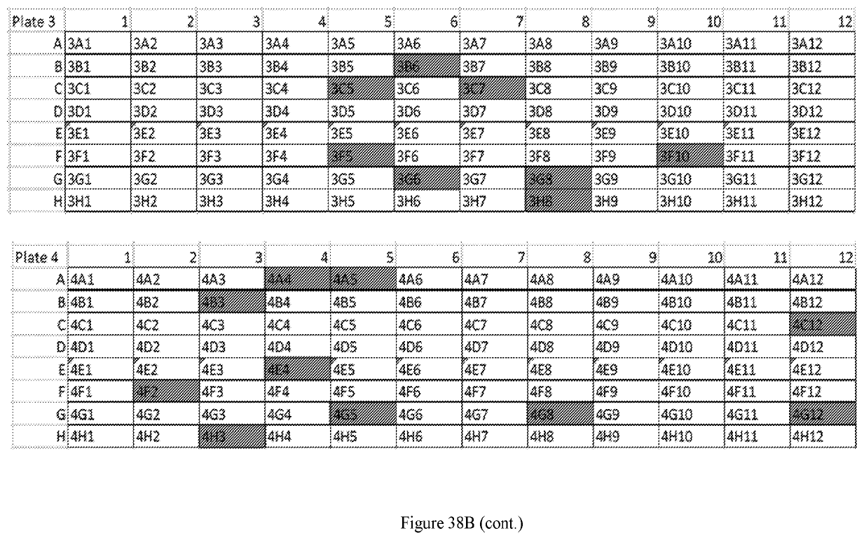

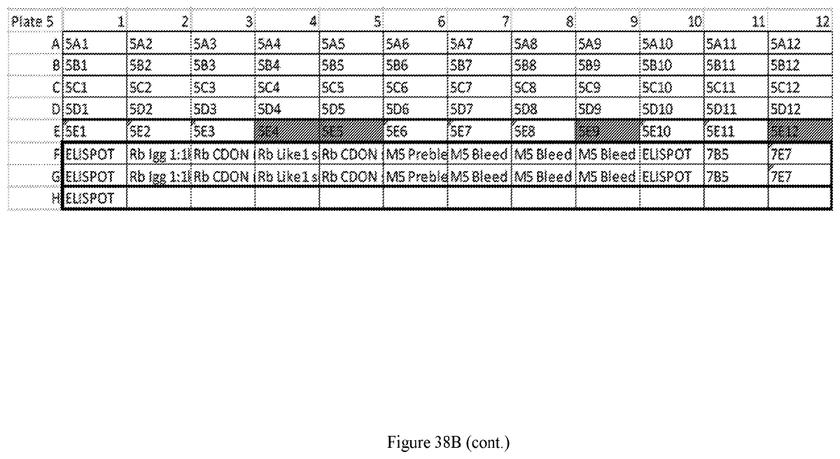

Fusion supernatants were screened by ELISA. A total of 5 plates with supernatants from 432 clones and 12 control wells were screened by ELISA. ELISA data from all plates were analyzed, a threshold was set, and 51 of 432 fusion wells were selected for expansion (see, FIGS. 38A and 38B). In particular, ELISA analysis was performed to detect reactivity of supernatants collected from mouse 5 splenocyte fusions to an immobilized OV-conjugated CDON peptide. Fused splenocytes were plated at low density (to obtain clonal populations) and five 96-well plates containing 432 individual wells of fused splenocytes were screened to detect highest reactivity to the peptide. ELISA results were scored (see, FIG. 38A; with highest scoring shaded blue). A total of 51 ‘hits’ were selected for expansion of the cells and further testing. The selected hits are indicated by red (high scores) and orange (intermediate scores) shaded boxes on the CLONE MAP (see, FIG. 38B).

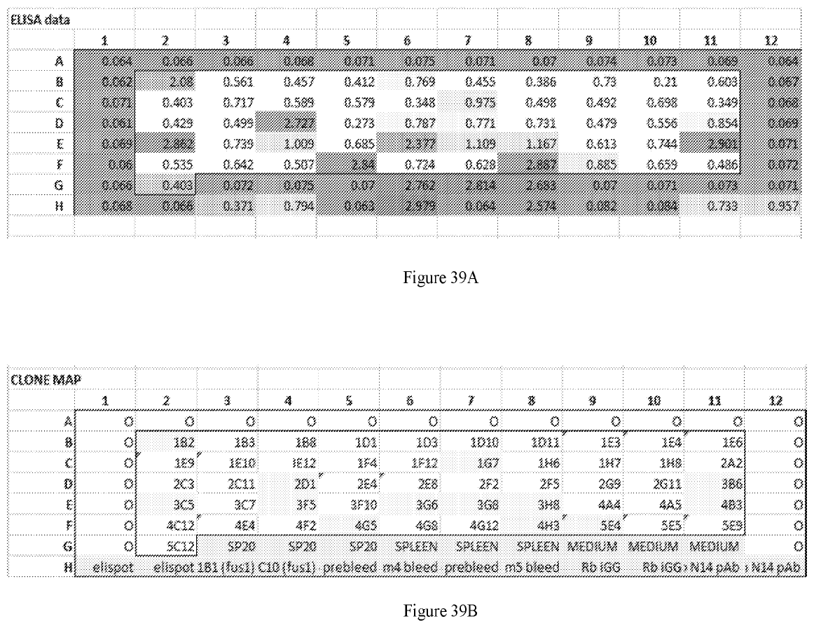

Cells in the 51 selected wells were expanded to 24 well plates for secondary screening. Supernatants from the 51 clones were tested in a second ELISA (see, FIGS. 39A and 39B). In particular, ELISA analysis was performed to detect reactivity of supernatants collected from the 51 ‘hits’ to an immobilized OV-conjugated CDON peptide. ELISA absorbance data is shown in FIG. 39A, and the corresponding clone map is shown in FIG. 39B, identifying the clones that were highest scoring by ELISA (yellow shaded cells).

Supermatants from the 51 clones were tested in cell sphere formation and cell viability assays. Combined results of the three assays were compared and 19 clones were selected and rank ordered based on high scores by ELISA and positive scores in cell-based sphere formation and viability assays (see, Table 1).

TABLE 1
Top 19 Clones from Fusion 2 Selected for further Analysis

| Clone ID | ELISA 1 Rank | ELISA 2 Rank | Cell-based Assay |
|---|---|---|---|
| 1B2 | 9 | 7 | intermediate |
| 1G7 | 2 | 11 | intermediate |
| 2D1 | 1 | 5 | intermediate |
| 3B6 | 35 | 13 | positive |
| 3C5 | 31 | 3 | positive |
| 3F5 | 39 | 10 | intermediate |
| 3G6 | 14 | 6 | positive |
| 3G8 | 29 | 9 | positive |
| 3H8 | 26 | 8 | intermediate |
| 4B3 | 20 | 1 | intermediate |
| 4G5 | 10 | 4 | intermediate |
| 4H3 | 16 | 2 | intermediate |
| 5E4 | 43 | 12 | intermediate |
| 2F5 | 28 | 19 | positive |
| 2G9 | 35 | 40 | positive |
| 4C12 | 45 | 34 | positive |
| 2A2 | 33 | 48 | positive |
| 1D3 | 3 | 16 | positive |
| 1E3 | 11 | 20 | positive |

These clones were then expanded and cryopreserved. Cryopreserved clones were prioritized for expansion and cloning by limiting dilution to ensure single clone purity.

Spleens were collected from the remaining immunized mice. Based on the high reactivity shown across all of the mice (mouse 1-5) after the second immunization with CDON peptide, splenocytes from the remaining four mice (mice #1-4; M1, M2, M3 and M4) were collected and cryopreserved. Mice M1-4 were boosted by intraperitoneal injection of CDON immunization peptide. Three days later, terminal bleeds and splenectomies were performed and single cell suspensions of the spleens of each of the four mice were prepared.

Spleens were picked up the same morning and splenocytes from each of the four mice were prepared for cryopreservation and banking.

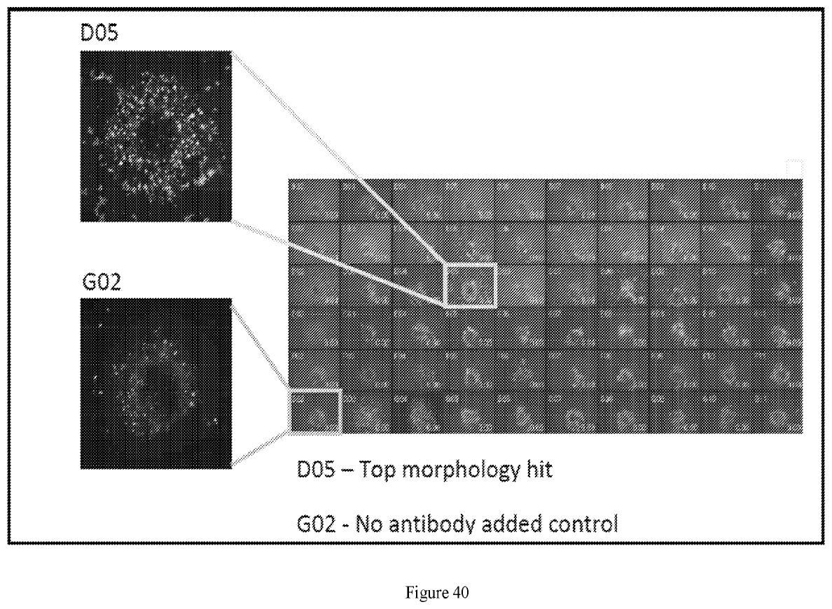

Cell based assays for all clones selected from Mouse 5 (M5) fusion #1 and fusion #2 were carried out. OVCAR-3 cells were cultivated and plated for assay. A new plate map was constructed and OVCAR-3 cells were treated with clone supernatants on the day of and day following cell plating. Cell supernatants from the 22 clones selected from fusion #1 and 19 clones selected from fusion #2, along with 5 borderline clones selected as controls, were tested in the cell sphere formation (see, FIG. 40) and cell viability assays. In particular, the effects of clone supernatants on OVCAR-3 cell morphology and viability were examined. Cells were plated in non-adherent plates and either treated with clone supernatants at the time of plating (Day 1) and 24 hours post plating (Day 2). Cells were stained with Hoescht and YoYo 1 (Day 5) and imaged. The size and shape of spheres were measured (morphology) and the scores for YoYo-1 staining and cell titer blue were obtained to measure cell viability was measured (Day 5 and Day 6 respectively). Enlarged images from well D05, treated with antibody containing supernatant from clone 12C4 (Fusion #1), and well control well G02, with no added antibody, are shown enlarged at left. Disruption of the sphere (enlarged and scattered morphology) and increased cell death marked by YoYo-1 staining are evident in the cells treated with the supernatant from clone 12C4.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1-32. (canceled)

33. A method of treating a human having a tumor comprising administering to the human in need thereof an antibody, or antigen-binding fragment thereof, that specifically binds Cell Adhesion Molecule-Related/Down-Regulated by Oncogenes (CDON) polypeptide, wherein the antibody, or antigen-binding fragment thereof, specifically binds:
a) a polypeptide consisting of amino acids at positions corresponding to positions 1 to 200 according to SEQ ID NO: 1; or
b) a polypeptide consisting of amino acids at positions corresponding to positions 1000 to 1287 according to SEQ ID NO: 1.

34. The method according to claim 33, wherein the antibody, or antigen-binding fragment thereof, specifically binds:
a) a polypeptide consisting of amino acids at positions corresponding to positions 100 to 200 according to SEQ ID NO: 1; or
b) a polypeptide consisting of amino acids at positions corresponding to positions 1200 to 1287 according to SEQ ID NO: 1.

35. The method according to claim 33, wherein the antibody, or antigen-binding fragment thereof, specifically binds:
a) a polypeptide consisting of amino acids at positions corresponding to positions 140 to 170 according to SEQ ID NO: 1; or
b) a polypeptide consisting of amino acids at positions corresponding to positions 1250 to 1287 according to SEQ ID NO: 1.

36. The method according to claim 33, wherein the antibody, or antigen-binding fragment thereof, specifically binds a peptide consisting of:
the amino acid sequence RVPESNPKAEVRYKIRGK (SEQ ID NO:2);
the amino acid sequence GIPLDSPTEV LQQPRET (SEQ ID NO: 3);
the amino acid sequence VLGDF GS STKHVITAEE (SEQ ID NO:4); or
the amino acid sequence KIRGKWLEHSTENY (SEQ ID NO:5).

37. The method according to claim 33, wherein the tumor is a brain tumor, an ovarian tumor, a pancreatic tumor, a breast tumor, a glioblastoma, a skin tumor, a meningioma, an astrocytoma, a liver tumor, a prostate carcinoma, a bladder tumor, a lung tumor, a lymphoma, a vascular endothelium hemangioma, a kidney carcinoma, or a thyroid follicular adenoma.

38. The method according to claim 33, wherein the tumor is an ovarian tumor or pancreatic tumor.

39. The method according to claim 36, wherein the tumor is a brain tumor, an ovarian tumor, a pancreatic tumor, a breast tumor, a glioblastoma, a skin tumor, a meningioma, an astrocytoma, a liver tumor, a prostate carcinoma, a bladder tumor, a lung tumor, a lymphoma, a vascular endothelium hemangioma, a kidney carcinoma, or a thyroid follicular adenoma.

40. The method according to claim 36, wherein the tumor is an ovarian tumor or pancreatic tumor.

41. The method of claim 33, wherein the tumor comprises a CDON-expressing tumor.

42. The method of claim 33, wherein administering the antibody, or antigen-binding fragment thereof, that specifically binds Cell Adhesion Molecule-Related/Down-Regulated by Oncogenes (CDON) polypeptide inhibits proliferation and induces cell death in a population of cancer cells.

43. The method of claim 33, wherein administering the antibody, or antigen-binding fragment thereof, that specifically binds Cell Adhesion Molecule-Related/Down-Regulated by Oncogenes (CDON) polypeptide inhibits adhesion in a population of cancer cells.

44. The method of claim 33, wherein administering the antibody, or antigen-binding fragment thereof, that specifically binds Cell Adhesion Molecule-Related/Down-Regulated by Oncogenes (CDON) polypeptide is repeated after one day, two days, three days, five days, one week, two weeks, three weeks, one month, five weeks, six weeks, seven weeks, eight weeks, two months or three months.

45. The method of claim 44, wherein repeated administration can be at the same dose or at a different dose and wherein administration can be repeated once, twice, three times, four times, five times, six times, seven times, eight times, nine times, ten times, or more.

46. The method of claim 33, wherein the human is an adult patient.

47. The method of claim 33, wherein the human is a pediatric patient.

48. The method of claim 33, wherein the antibody, or antigen-binding fragment thereof, that specifically binds Cell Adhesion Molecule-Related/Down-Regulated by Oncogenes (CDON) polypeptide is provided in a composition comprising at least one pharmaceutically acceptable carrier and, optionally, one or more additional therapeutic agents.

49. The method of claim 48, wherein the composition is administered to the human orally, transdermally, subcutaneously, intranasally, intravenously, intraarterially, intramuscularly, intraocularly, topically, locally, intrathecally, intracerebroventricularly, intraspinally, or inracranially.

50. The method of claim 48, wherein the composition is presented in unit dose forms containing a predetermined amount of anti-CDON antibodies, or antigen-binding fragments thereof per dose and wherein the unit dose comprises about 0.1 mg to about 5 g, about 1 mg to about 1 g, or about 10 to about 50 mg.


SEQUENCE LISTING (5 sequences)

SEQ ID NO: 1 - protein, 1287 aa, Homo sapiens
MHPDLGPLCTLLYVTLTILCSSVSSDLAPYFTSEPLSAVQKLGGPVVLHCSAQPVTTRISWLHNGKTLDGNLEHVKIHQGTLTILSLNSSLLGYYQCLANNSIGAIVSGPATVSVAVLGDFGSSTKHVITAEEKSAGFIGCRVPESNPKAEVRYKIRGKWLEHSTENYLILPSGNLQILNVSLEDKGSYKCAAYNPVTHQLKVEPIGRKLLVSRPSSDDVHILHPTHSQALAVLSRSPVTLECVVSGVPAPQVYWLKDGQDIAPGSNWRRLYSHLATDSVDPADSGNYSCMAGNKSGDVKYVTYMVNVLEHASISKGLQDQIVSLGATVHFTCDVHGNPAPNCTWFHNAQPIHPSARHLTAGNGLKISGVTVEDVGMYQCVADNGIGFMHSTGRLEIENDGGFKPVIITAPVSAKVADGDFVTLSCNASGLPVPVIRWYDSHGLITSHPSQVLRSKSRKSQLSRPEGLNLEPVYFVLSQAGASSLHIQAVTQEHAGKYICEAANEHGTTQAEASLMVVPFETNTKAETVTLPDAAQNDDRSKRDGSETGLLSSFPVKVHPSAVESAPEKNASGISVPDAPIILSPPQTHTPDTYNLVWRAGKDGGLPINAYFVKYRKLDDGVGMLGSWHTVRVPGSENELHLAELEPSSLYEVLMVARSAAGEGQPAMLTFRTSKEKTASSKNTQASSPPVGIPKYPVVSEAANNNFGVVLTDSSRHSGVPEAPDRPTISTASETSVYVTWIPRANGGSPITAFKVEYKRMRTSNWLVAAEDIPPSKLSVEVRSLEPGSTYKFRVIAINHYGESFRSSASRPYQVVGFPNRFSSRPITGPHIAYTEAVSDTQIMLKWTYIPSSNNNTPIQGFYIYYRPTDSDNDSDYKRDVVEGSKQWHMIGHLQPETSYDIKMQCFNEGGESEFSNVMICETKVKRVPGASEYPVKDLSTPPNSLGSGGNVGPATSPARSSDMLYLIVGCVLGVMVLILMVFIAMCLWKNRQQNTIQKYDPPGYLYQGSDMNGQMVDYTTLSGASQINGNVHGGFLTNGGLSSGYSHLHHKVPNAVNGIVNGSLNGGLYSGHSNSLTRTHVDFEHPHHLVNGGGMYTAVPQIDPLECVNCRNCRNNNRCFTKTNSTFSSSPPPVVPVVAPYPQDGLEMKPLSHVKVPVCLTSAVPDCGQLPEESVKDNVEPVPTQRTCCQDIVNDVSSDGSEDPAEFSRGQEGMINLRIPDHLQLAKSCVWEGDSCAHSETEINIVSWNALILPPVPEGCAEKTMWSPPGIPLDSPTEVLQQPRET

SEQ ID NO: 2 - protein, 18 aa, synthetic construct
  REGION 1..18: note = CDON peptide
RVPESNPKAEVRYKIRGK

SEQ ID NO: 3 - protein, 17 aa, synthetic construct
  REGION 1..17: note = CDON peptide
GIPLDSPTEVLQQPRET

SEQ ID NO: 4 - protein, 17 aa, synthetic construct
  REGION 1..17: note = CDON peptide
VLGDFGSSTKHVITAEE

SEQ ID NO: 5 - protein, 14 aa, synthetic construct
  REGION 1..14: note = CDON peptide
KIRGKWLEHSTENY